(19) United States
(12) Patent Application Publication
(10) Pub. No.: US 2019/0202774 A1
(43) Pub. Date: Jul. 4, 2019

(54) BIARYL COMPOUNDS AS ANTIMICROBIAL AND CHEMOTHERAPEUTIC AGENTS

(71) Applicants: WILLIAM MARSH RICE UNIVERSITY, Houston, TX (US); THE TEXAS A&M UNIVERSITY SYSTEM, College Station, TX (US); THE BOARD OF REGENTS OF THE UNIVERSITY OF TEXAS SYSTEM, Austin, TX (US)

(72) Inventors: Hongyin GAO, Houston, TX (US); Laszlo KURTI, Bellaire, TX (US); Kush SHAH, College Station, TX (US); Parth SHAH, College Station, TX (US); Carolyn L CANNON, College Station, TX (US)

(73) Assignees: WILLIAM MARSH RICE UNIVERSITY, Houston, TX (US); THE TEXAS A&M UNIVERSITY SYSTEM, College Station, TX (US); THE BOARD OF REGENTS OF THE UNIVERSITY OF TEXAS SYSTEM, Austin, TX (US)

(21) Appl. No.: 15/776,035

(22) PCT Filed: Nov. 11, 2016
(86) PCT No.: PCT/US2016/061607

§ 371 Date: Feb. 26, 2019

Related U.S. Application Data
(60) Provisional application No. 62/255,254, filed on Nov. 13, 2015.

(51) Int. Cl. C07C 217/84; A61P 31/04; C07C 215/74; A61P 35/00

(52) U.S. Cl. CPC C07C 217/84; A61P 31/04; C07C 39/367; A61P 35/00; C07C 215/74

(57) ABSTRACT

In one aspect, the present disclosure provides diaryl compounds of the formula presented herein. The application also provides compositions and methods of treatment thereof. In some embodiments, these compounds are used in the treatment of bacterial infections or in the treatment of cancer.

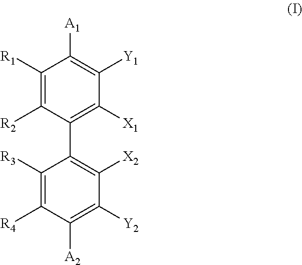

This application claims the benefit of U.S. Provisional Application No. 62/255,254 filed on Nov. 13, 2015, the entirety of which is hereby incorporated by reference.

### BACKGROUND

### 1. Field

This disclosure relates to the fields of medicine, pharmacology, chemistry, antimicrobial activity, and oncology. In particular, new compounds, compositions, and methods of treatment comprising a biaryl core are disclosed.

### 2. Related Art

Infectious diseases are currently the second leading cause of death worldwide and the third leading cause of death in economically advanced countries despite the development of antibiotics (Nathan, 2004). Furthermore, the threatening emergence of bacterial resistance (US Department of Health and Human Services, Center of Disease Control and Prevention, Antibiotic Resistance Threats in the United States, 2013, www.cdc.gov/drugresistance/threat-report-2013/pdf/ar-threats-2013-508.pdf.) to many antibiotics presents a serious challenge for their clinical use. According to the 2013 estimates published by the Center for Disease Control (CDC), over two million people acquire serious drug resistant infections annually, resulting in over 23,000 fatalities (CDC www.cdc.gov/drugresistance/threat-report-2013/:, 2013). These multi-drug resistant bacteria are classified into three categories—urgent, serious, and concerning, by the CDC. A serious threat level has been assigned to MRSA, vancomycin-resistant Enterococcus (VRE), drug resistant Streptococcus pneumoniae, and drug resistant tuberculosis [10]. These four pathogens lead to approximately 1.3 million cases of infection and 19,000 fatalities annually (CDC www.cdc.gov/drugresistance/threat-report-2013/:, 2013).

Kamei and Isnansetyo reported antimicrobial activity of bromophene (also known as MC21-A, and represented as C58 here), a biaryl compound in 2003 (Isnansetyo and Kamei, 2003). Bromophene is one of the three natural metabolites; MC21-A, —B, and —C isolated and purified from a marine bacterium Pseudoalteromonas phenolica (Isnansetyo and Kamei, 2009). However, the process reported by Kamei and Isnansetyo (Isnansetyo and Kamei, 2003 and Isnansetyo and Kamei, 2009) requires arduous purification to yield miniscule quantity of the final product and is not cost effective and poses major logistical problems. Recently, the Kurd laboratory identified and developed an economically viable, unique set of reactions and catalysts to synthesize and modify these biaryl compounds in a single step. Therefore, there is to use these methods to develop new antibiotic agents.

### SUMMARY

Thus, the present disclosure provides compounds of the formula:

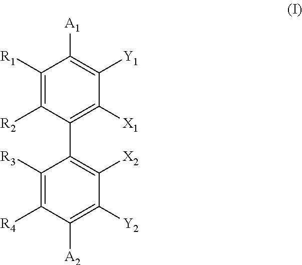

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
Y₂ is hydrogen or halo;
A₁ and A₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₁ and R₂ are taken together and are alkenediyl_{(C≤8)} or substituted alkenediyl_{(C≤8)}; and
R₃ and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₃ and R₄ are taken together and are alkenediyl_{(C≤8)} or substituted alkenediyl_{(C≤8)}; or
   compounds of the formula:

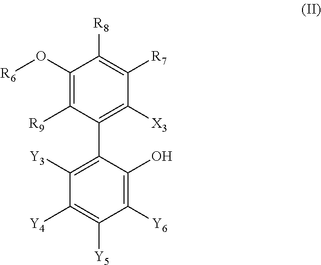

wherein:
X₃ is —OR_{d} or —NRₑR_{f}, wherein:
   R_{d} is hydrogen, alkyl_{(C≤8)}, cycloalkyl_{(C≤8)}, acyl_{(C≤8)}, or a substituted version of any of these groups; or a hydroxy protecting group;
   Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
   or a pharmaceutically acceptable salt thereof.

In some embodiments, the formula is further defined as:

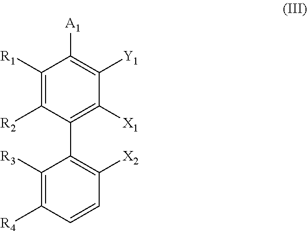

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₁ and R₂ are taken together and are alkenediyl_{(C≤8)} or substituted alkenediyl_{(C≤8)}; and
R₃ and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₃ and R₄ are taken together and are alkenediyl_{(C≤8)} or substituted alkenediyl_{(C≤8)};
   or a pharmaceutically acceptable salt thereof.

In some embodiments, the formula is further defined as:

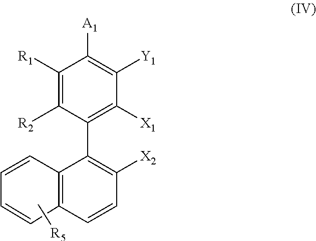

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; or R₁ and R₂ are taken together and are alkenediyl_{(C≤8)} or substituted alkenediyl_{(C≤8)}; and
R₅ is hydrogen, halo, methoxy, ethoxy, methyl, or ethyl;
   or a pharmaceutically acceptable salt thereof.

In some embodiments, the formula is further defined as:

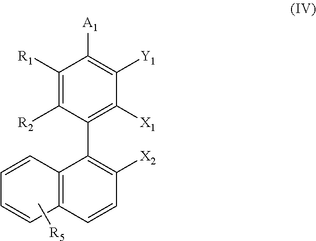

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, or substituted cycloalkyl_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; and
R₅ is hydrogen, halo, methoxy, ethoxy, methyl, or ethyl;
   or a pharmaceutically acceptable salt thereof.

In some embodiments, the formula is further defined as:

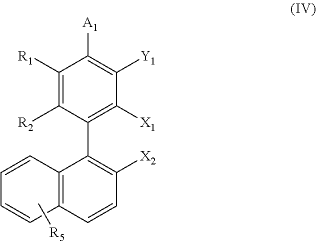

wherein:
X₁ is amino or alkylamino_{(C≤8)}, substituted alkylamino_{(C≤8)}, dialkylamino_{(C≤8)}, or substituted dialkylamino_{(C≤8)};
X₂ is hydroxy;
Y₁ is halo;
A₁ is hydrogen, halo, alkyl_{(C≤8)}, substituted alkyl_{(C≤8)}, cycloalkyl_{(C≤8)}, or substituted cycloalkyl_{(c≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; and
R₅ is hydrogen, halo, methoxy, ethoxy, methyl, or ethyl;
   or a pharmaceutically acceptable salt thereof.

In other embodiments, the formula is further defined as:

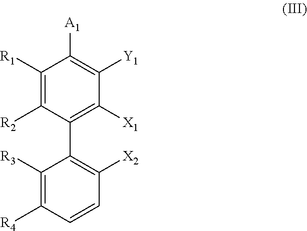

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, R₃, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
   or a pharmaceutically acceptable salt thereof.

In some embodiments, the formula is further defined as:

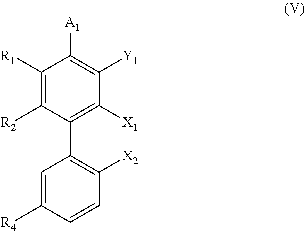

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
   or a pharmaceutically acceptable salt thereof.

In some embodiments, the formula is further defined as:

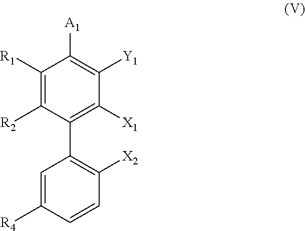

wherein:
X₁ is amino or alkylamino_{(C≤8)}, substituted alkylamino_{(C≤8)}, dialkylamino_{(C≤8)}, or substituted dialkylamino_{(C≤8)};
X₂ is hydroxy;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
   or a pharmaceutically acceptable salt thereof.

In some embodiments, the formula is further defined as:

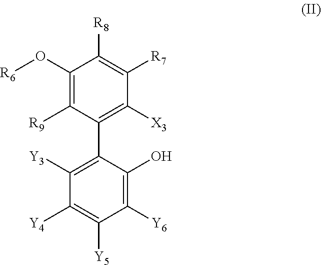

wherein:
X₃ is —OR_{d} or —NRₑR_{f}, wherein:
   R_{d} is hydrogen, alkyl_{(C≤8)}, cycloalkyl_{(C≤8)}, acyl_{(C≤8)}, or a substituted version of any of these groups; or a hydroxy protecting group;
   Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
   or a pharmaceutically acceptable salt thereof.

In some embodiments, the formula is further defined as:

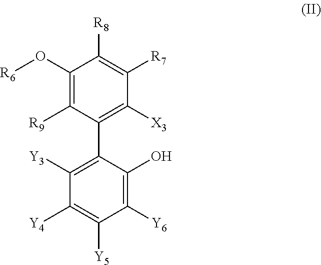

wherein:
X₃ is —OR_{d} or —NRₑR_{f}, wherein:
   R_{d} is hydrogen, alkyl_{(C≤8)}, cycloalkyl_{(C≤8)}, acyl_{(C≤8)}, or a substituted version of any of these groups; or a hydroxy protecting group;
   Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
   or a pharmaceutically acceptable salt thereof.

In some embodiments, the formula is further defined as:

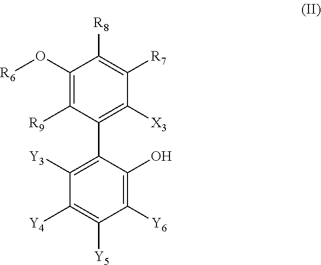

wherein:
X₃ is —OR_{d} or —NRₑR_{f}, wherein:
   R_{d} is hydrogen, alkyl_{(C≤8)}, cycloalkyl_{(C≤8)}, acyl_{(C≤8)}, or a substituted version of any of these groups;
   Rₑ and R_{f} are each independently hydrogen, alkyl_{(C≤8)}, cycloalkyl_{(C≤8)}, acyl_{(C≤8)}, or a substituted version of any of these groups;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)};
   or a pharmaceutically acceptable salt thereof.

In some embodiments, X₁ is amino. In some embodiments, X₂ is hydroxy. In some embodiments, A₁ is hydrogen. In other embodiments, A₁ is halo such as fluoro, chloro, or bromo. In other embodiments, A₁ is alkyl_{(C≤8)} or substituted alkyl_{(C≤8)}. In some embodiments, A₁ is alkyl_{(C≤8)} such as methyl.

In some embodiments, A₂ is hydrogen. In other embodiments, A₂ is halo such fluoro, chloro, or bromo. In other embodiments, A₂ is alkyl_{(C≤8)} or substituted alkyl_{(C≤8)}. In some embodiments, A₂ is alkyl_{(C≤8)} such as methyl.

In some embodiments, R₁ is hydrogen. In other embodiments, R₁ is halo such fluoro, chloro, or bromo. In other embodiments, R₁ is alkyl_{(C≤8)} or substituted alkyl_{(C≤8)}. In some embodiments, R₁ is alkyl_{(C≤8)} such as methyl.

In some embodiments, R₂ is hydrogen. In other embodiments, R₂ is halo such fluoro, chloro, or bromo. In other embodiments, R₂ is alkyl_{(C≤8)} or substituted alkyl_{(C≤8)}. In some embodiments, R₂ is alkyl_{(C≤8)} such as methyl. In other embodiments, R₂ is alkoxy_{(C≤8)} or substituted alkoxy_{(C≤8)}. In some embodiments, R₂ is alkoxy_{(C≤8)} such as methoxy.

In some embodiments, R₃ is hydrogen. In other embodiments, R₃ is taken together with R₄ and are alkenediyl_{(C≤8)} or substituted alkenediyl_{(C≤8)}.

In some embodiments, R₄ is hydrogen. In other embodiments, R₄ is halo such fluoro, chloro, or bromo. In other embodiments, R₄ is alkyl_{(C≤8)} or substituted alkyl_{(C≤8)}. In some embodiments, R₄ is alkyl_{(C≤8)} such as methyl. In other embodiments, R₄ is alkoxy_{(C≤8)} or substituted alkoxy_{(C≤8)}. In some embodiments, R₄ is alkoxy_{(C≤8)} such as methoxy.

In some embodiments, Y₂ is hydrogen. In some embodiments, R₅ is methoxy or ethoxy. In some embodiments, R₅ is methoxy.

In some embodiments, X₃ is —OR_{d}, wherein: R_{d} is hydrogen, alkyl_{(C≤8)}, cycloalkyl_{(C≤8)}, acyl_{(C≤8)}, or a substituted version of any of these groups. In some embodiments, X₃ is —OH. In other embodiments, X₃ is —NRₑR_{f}, wherein: Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group. In some embodiments, Rₑ is hydrogen. In other embodiments, Rₑ is a monovalent amino protecting group such as a mesyl or toslyl group. In some embodiments, R_{f} is hydrogen.

In some embodiments, R₆ is alkyl_{(C≤8)} or substituted alkyl_{(C≤8)}. In some embodiments, R₆ is alkyl_{(C≤8)} such as methyl.

In some embodiments, R₇ is hydrogen. In other embodiments, R₇ is halo such fluoro, chloro, or bromo.

In some embodiments, R₈ is hydrogen. In other embodiments, R₈ is halo such fluoro, chloro, or bromo. In other embodiments, R₈ is alkyl_{(C≤8)} or substituted alkyl_{(C≤8)}. In some embodiments, R₈ is alkyl_{(C≤8)} such as methyl. In other embodiments, R₈ is alkoxy_{(C≤8)} or substituted alkoxy_{(C≤8)}. In some embodiments, R₈ is alkoxy_{(C≤8)} such as methoxy.

In some embodiments, R₉ is hydrogen. In other embodiments, R₉ is halo such fluoro, chloro, or bromo. In other embodiments, R₉ is alkyl_{(C≤8)} or substituted alkyl_{(C≤8)}. In some embodiments, R₉ is alkyl_{(C≤8)} such as methyl. In other embodiments, R₉ is alkoxy_{(C≤8)} or substituted alkoxy_{(C≤8)}. In some embodiments, R₉ is alkoxy_{(C≤8)} such as methoxy.

In some embodiments, Y₃ is hydrogen. In other embodiments, Y₃ is hydroxy. In other embodiments, Y₃ is halo such as chloro or bromo. In other embodiments, Y₃ is alkyl_{(C≤8)} or substituted alkyl_{(C≤8)}. In some embodiments, Y₃ is alkyl_{(C≤8)} such as methyl. In other embodiments, Y₃ is alkoxy_{(C≤8)} or substituted alkoxy_{(C≤8)}. In some embodiments, Y₃ is alkoxy_{(C≤8)} such as methoxy.

In some embodiments, Y₄ is hydrogen. In other embodiments, Y₄ is hydroxy. In other embodiments, Y₄ is halo such as chloro or bromo. In other embodiments, Y₄ is alkyl_{(C≤8)} or substituted alkyl_{(C≤8)}. In some embodiments, Y₄ is alkyl_{(C≤8)} such as methyl. In other embodiments, Y₄ is alkoxy_{(C≤8)} or substituted alkoxy_{(C≤8)}. In some embodiments, Y₄ is alkoxy_{(C≤8)} such as methoxy.

In some embodiments, Y₅ is hydrogen. In other embodiments, Y₅ is hydroxy. In other embodiments, Y₅ is halo such as chloro or bromo. In other embodiments, Y₅ is alkyl_{(C≤8)} or substituted alkyl_{(C≤8)}. In some embodiments, Y₅ is alkyl_{(C≤8)} such as methyl. In other embodiments, Y₅ is alkoxy_{(C≤8)} or substituted alkoxy_{(C≤8)}. In some embodiments, Y₅ is alkoxy_{(C≤8)} such as methoxy.

In some embodiments, Y₆ is hydrogen. In other embodiments, Y₆ is hydroxy. In other embodiments, Y₆ is halo such as chloro or bromo. In other embodiments, Y₆ is alkyl_{(C≤8)} or substituted alkyl_{(C≤8)}. In some embodiments, Y₆ is alkyl_{(C≤8)} such as methyl. In other embodiments, Y₆ is alkoxy_{(C≤8)} or substituted alkoxy_{(C≤8)}. In some embodiments, Y₆ is alkoxy_{(C≤8)} such as methoxy.

In some embodiments, the compounds are further defined as:

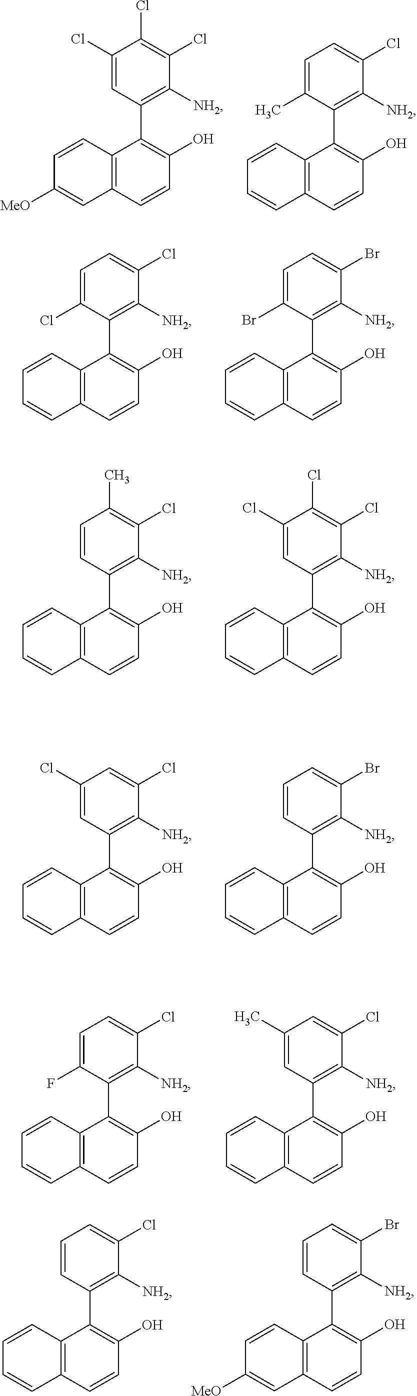

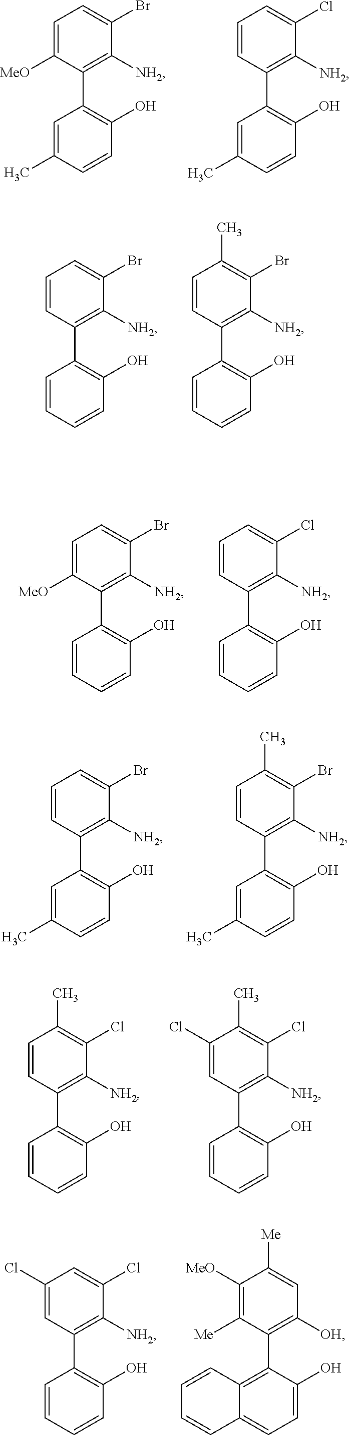

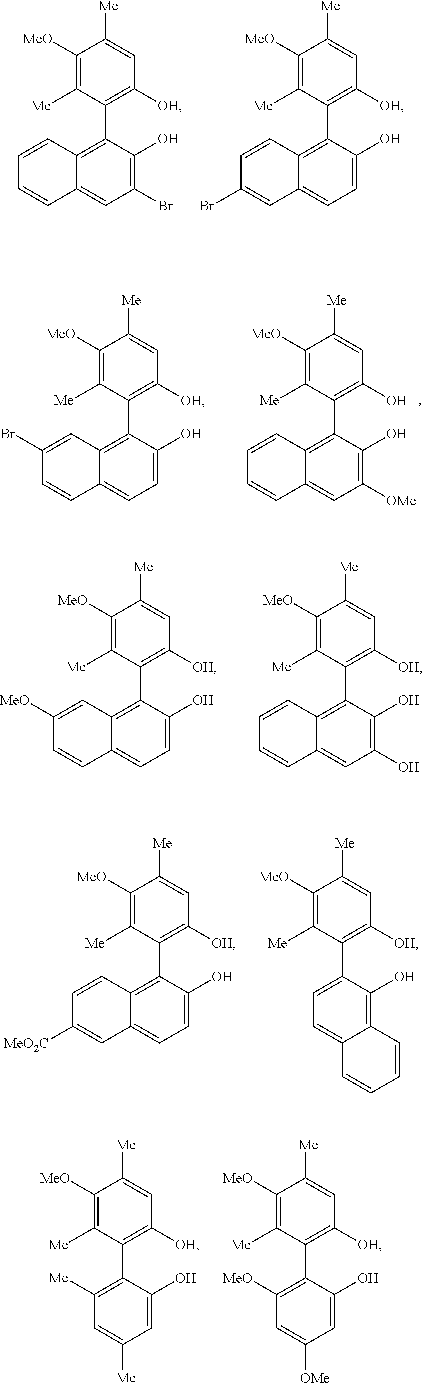

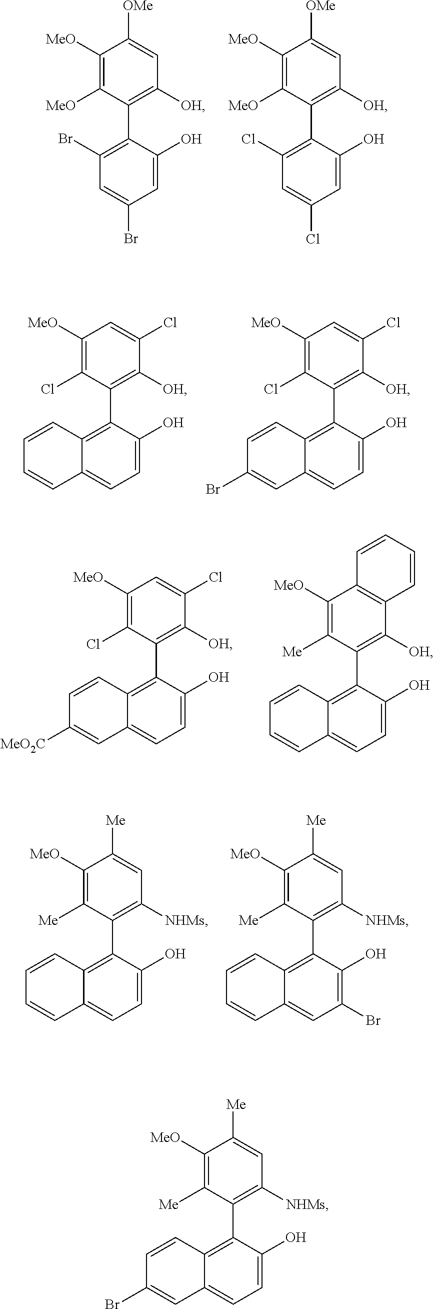

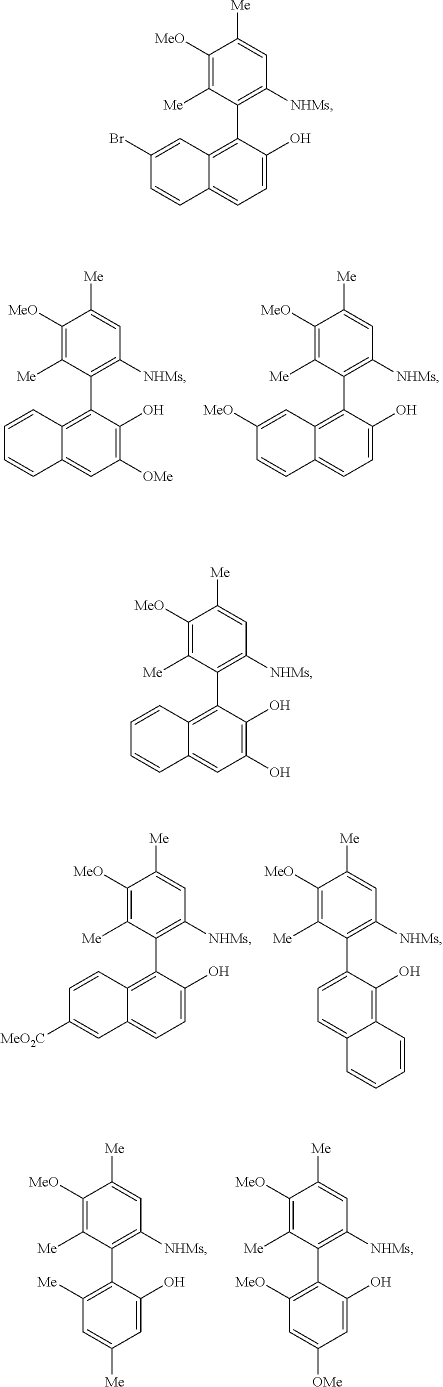

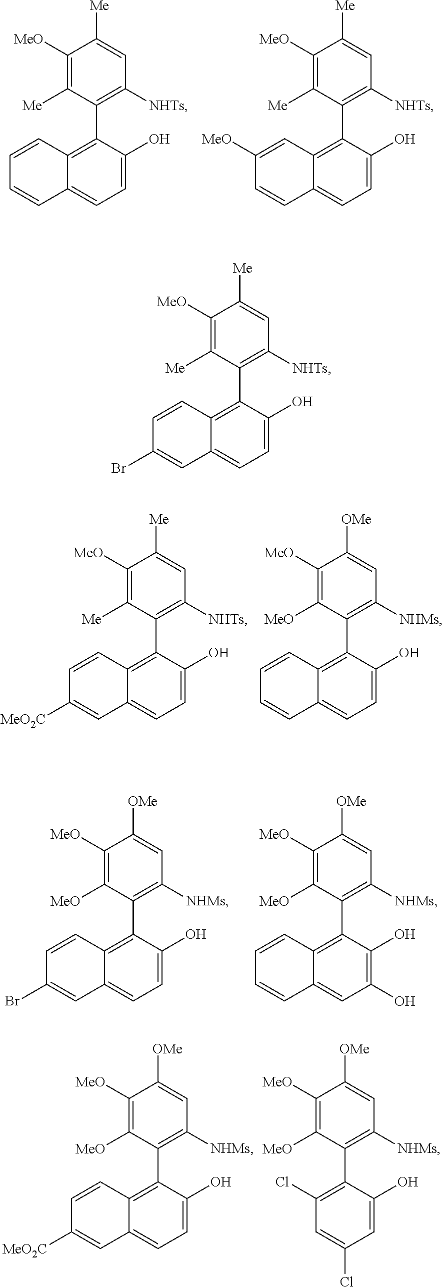

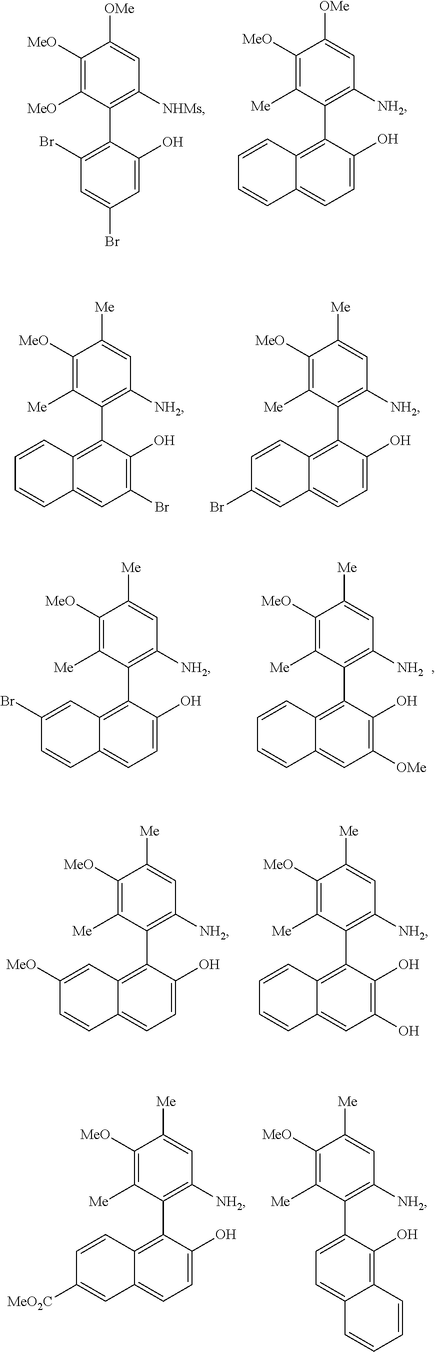

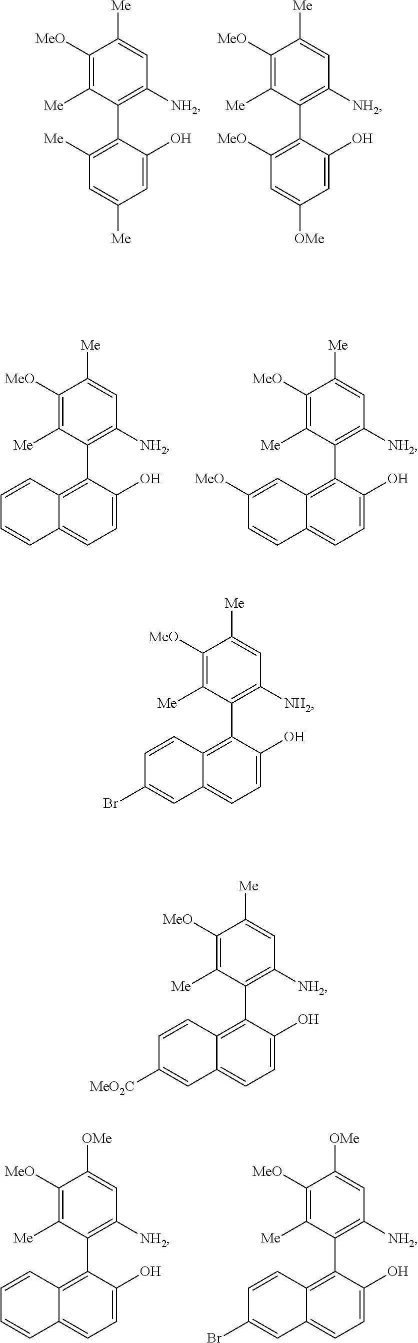

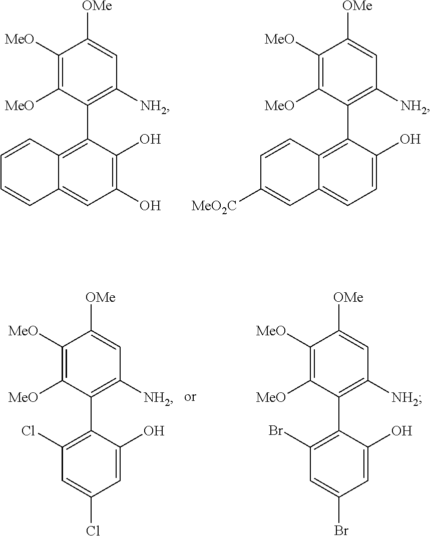 [SMILES: COc1cc(N)c(-c2c(O)c(O)cc3ccccc23)c(OC)c1OC ; COC(=O)c1ccc2c(-c3c(N)cc(OC)c(OC)c3OC)c(O)ccc2c1 ; COc1cc(N)c(-c2c(O)cc(Cl)cc2Cl)c(OC)c1OC ; COc1cc(N)c(-c2c(O)cc(Br)cc2Br)c(OC)c1OC]

or a pharmaceutically acceptable salt thereof.

In yet another aspect, the present disclosure provides pharmaceutical compositions comprising:

(a) a compound described herein; and
(b) a pharmaceutically acceptable carrier.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical compositions are formulated for intravenous administration, oral administration, topical administration, or administration as an aerosol. In some embodiments, the aerosols are formulated as a dry powder or as a solution. In some embodiments, the compound is formulated using an electrospun membrane. In some embodiments, the compound is encapsulated in a liposome, micelle, or polymeric nanoparticle. In some embodiments, the pharmaceutical composition is formulated as a unit dose.

In yet another aspect, the present disclosure provides pharmaceutical compositions comprising:

(a) a compound of the formula:

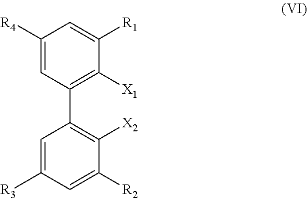

wherein:
R₁, R₂, R₃, and R₄ are each halo;
X₁ and X₂ are OAc or O⁻M⁺; wherein:
   M⁺ is a monovalent cations or the M⁺ associated with X₁ and X₂ are taken together and are a divalent cation; and
      (b) a pharmaceutically acceptable carrier.

In some embodiments, M⁺ is a monovalent cation such as a cation of lithium, sodium, potassium, or rubidium. In some embodiments, R₁, R₂, R₃, and R₄ are all the same halo. In some embodiments, R₁, R₂, R₃, and R₄ are all chloro. In other embodiments, R₁, R₂, R₃, and R₄ are all bromo.

In some embodiments, the compounds are further defined as:

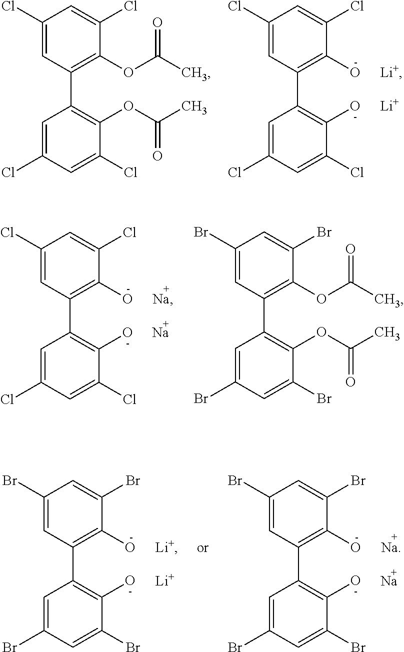

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical compositions are formulated for intravenous administration, oral administration, topical administration, or administration as an aerosol. In some embodiments, the aerosols are formulated as a dry powder or as a solution. In some embodiments, the compound is formulated using an electrospun membrane. In some embodiments, the compound is encapsulated in a liposome, micelle, or polymeric nanoparticle. In some embodiments, the pharmaceutical composition is formulated as a unit dose.

In still yet another aspect, the present disclosure provides methods of treating a disease or disorder in a patient comprising administering to the patient in need thereof a therapeutically effective amount of a compound or composition described herein.

In some embodiments, the disease or disorder is a bacterial infection. In some embodiments, the bacterial infection is an infection of gram positive bacterium. In some embodiments, the bacterial infection is an infection of a pathogenic gram positive bacterium. In some embodiments, the pathogenic gram positive bacterium is a plant, animal, or human pathogen.

In some embodiments, the pathogenic gram positive bacterium is an animal or human pathogen. In some embodiments, the pathogenic gram positive bacterium is a Streptococcus, Staphylococcus, Corynebacterium, Listeria, Bacillus, Enterococcus, Nocardia, or Clostridium bacterium. In some embodiments, the Staphylococcus bacterium is Staphylococcus aureus such as methicillin resistant Staphylococcus aureus. In some embodiments, the Enterococcus bacterium is Enterococcus faecalis.

In other embodiments, the pathogenic gram positive bacterium is a plant pathogen. In some embodiments, the pathogenic gram positive bacterium is a Rathybacter, Leifsonia, or Clavibacter bacterium.

In other embodiments, the bacterial infection is an infection of acid fast bacterium. In some embodiments, the acid fast bacterium is a human or animal pathogen such as Mycobacterium or Rhodococcus bacterium. In some embodiments, the Mycobacterium bacterium is Mycobacterium tuberculosis or a non-tuberculous Mycobacterium. In some embodiments, the Rhodococcus bacterium is Rhodococcus equi. In other embodiments, the bacterial infection is an infection of a gram negative bacterium.

In some embodiments, the bacterial infection exhibits resistance to one or more common antibiotics. In some embodiments, the bacterial infection exhibits resistance to multiple different antibiotics. In some embodiments, the method further comprises administering one or more antibiotic agents.

In other embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the cancer is lung cancer. In some embodiments, the lung cancer is a non-small cell lung cancer. In other embodiments, the lung cancer is a large cell lung cancer. In some embodiments, the method comprises administering a second cancer therapy. In some embodiments, the second cancer therapy is a second chemotherapeutic agent, surgery, a radiotherapy, or an immunotherapy.

In some embodiments, the patient is a plant. In other embodiments, the patient is an animal. In some embodiments, the patient is a mammal such as a human. In some embodiments, the method comprises administering the compound or composition once. In some embodiments, the method comprises administering the compound or composition two or more times.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1—Chemical structures of biaryl compounds tested for biological activity as described in Example 5.

FIG. 2—Antimicrobial activity and toxicity of biaryl compounds and precursor molecules tested against MRSA strain USA300 and human lung epithelial 16HBE cells, respectively.

FIGS. 3A-3F—Minimum inhibitory and minimum bactericidal concentrations of (FIG. 3A) C58, (FIG. 3B) C59, (FIG. 3C) vancomycin hydrochloride, (FIG. 3D) clindamycin hydrochloride, (FIG. 3E) daptomycin hydrochloride, and (FIG. 3F) linezolid against 38 MRSA strains determined using standard broth microdilution method. Data presented are from studies performed in triplicate.

FIGS. 4A-4B—Bacterial burden at 1, 2, 4, 8, 12, and 24 hours upon incubation of log-phase MRSA strain USA300 with (FIG. 4A) C59 and (FIG. 4B) Vancomycin hydrochloride.

FIGS. 5A-5D—Bacterial burden at 4, 8, and 24 hours upon incubation with (FIG. 5A, FIG. 5C) C59 and (FIG. 5B, FIG. 5D) vancomycin hydrochloride upon incubation with stationary-phase MRSA strains (FIG. 5A, FIG. 5B) USA300 and (FIG. 5C, FIG. 5D) SALL06.

FIGS. 6A-6B—Colony forming units per treatment (CFU/peg) of MRSA strain (FIG. 6A) SAD05 and (FIG. 6B) 0632 bacteria in a biofilm after a six incubation with biaryl compounds C58, or C59, or control vancomycin hydrochloride.

FIGS. 7A-7D—Chemical structures of (FIG. 7A) C59, (FIG. 7B) acetylated C59 (C59-2), (FIG. 7C) sodium salt of C59 (C59Na), and (FIG. 7D) lithium salt of C59 (C59Li).

FIGS. 8A-8D—Antimicrobial activity of (FIG. 8A) C59 and its variants (FIG. 8B) acetylated C59 (C59-2), (FIG. 8C) sodium salt of C59 (C59Na), and (FIG. 8D) lithium salt of C59 (C59Li) against six strains of MRSA.

FIGS. 9A-9F—Bacteriostatic activity of biaryl compounds, C58, C59, C59Na, C59Li, C59-2, and control vancomycin hydrochloride at pH 7.4, 6.5, and 5.5 against MRSA strains (FIG. 9A) USA300, (FIG. 9B) 0606, (FIG. 9C) 0608, (FIG. 9D) 0634, (FIG. 9E) 0641, and (FIG. 9F) 0646 determined using standard broth microdilution method.

FIG. 10A-10F—Bactericidal activity of biaryl compounds, C58, C59, C59Na, C59Li, C59-2, and control vancomycin hydrochloride at pH 7.4, 6.5, and 5.5 against MRSA strains (FIG. 10A) USA300, (FIG. 10B) 0606, (FIG. 10C) 0608, (FIG. 10D) 0634, (FIG. 10E) 0641, and (FIG. 10F) 0646 determined using standard broth microdilution method.

FIG. 11—Fluorescence signal from DiSC₃(5) released as a result of membrane permeabilization of MRSA strain USA300 upon incubation with biaryl compound C58 compared to positive control, 100 nM 3,3′,4′,5-tetrachlorosalicylanilide, a known membrane permeabilization agent.

FIG. 12—Hemolytic activity of biaryl compounds after a 18-hour incubation with erythrocytes.

FIGS. 13A-13C—Toxicity of biaryl compounds against mammalian cell lines (FIG. 13A) human dermal fibroblasts, (FIG. 13B) 16HBE human bronchial epithelial, and (FIG. 13C) J774.A₁ murine macrophages after a 24 hour incubation determined using an alamarBlue® Cell Viability Assay.

FIGS. 14—Chemical structures of biaryl compounds evaluated to identify lead antimicrobial candidates with activity against Gram-positive pathogens.

FIG. 15—Antimicrobial activity of biaryls tested against representative strains of MRSA (USA 300), S. epidermidis (M0881), S. pyogenes (NR33709), and S. pneumoniae (TCH8431).

FIGS. 16A & 16B—Minimum inhibitory and bactericidal concentrations (MIC and MBC) of (FIG. 16A) 4-76 and (FIG. 16B) 5-32 against 38 MRSA strains using standard CLSI broth microdilution method. Data presented are from studies performed in triplicate.

FIGS. 17A-17D—Minimum inhibitory and bactericidal concentrations (MIC and MBC) of (FIG. 17A) C58, (FIG. 17B) C59, (FIG. 17C) 4-76, and (FIG. 17D) vancomycin hydrochloride against 21 S. epidermidis strains using standard CLSI broth microdilution method. Data presented are from studies performed in triplicate.

FIGS. 18A-18C—Minimum inhibitory and bactericidal concentrations (MIC and MBC) of (FIG. 18A) C59, (FIG. 18B) 4-76, and (FIG. 18C) 5-32 against 7 S. pyogenes strains using standard CLSI broth microdilution method. Data presented are from studies performed in duplicate.

FIGS. 19A-19C—Minimum inhibitory and bactericidal concentrations (MIC and MBC) of (FIG. 19A) 4-76, (FIG. 19B) 5-32, and (FIG. 19C) vancomycin hydrochloride against 6-7 S. pneumoniae strains using standard CLSI broth microdilution method. Data presented are from studies performed in duplicate.

FIGS. 20A-20D—Minimum inhibitory and bactericidal concentrations (MIC and MBC) of (FIG. 20A) C58Na with 2.5% DMSO, (FIG. 20B) C58Na without 2.5% DMSO, and (FIG. 20C) C59Na without 2.5% DMSO, and (FIG. 20D) C59Li without 2.5% DMSO against 6 MRSA strains using standard CLSI broth microdilution method. Data presented are from studies performed in duplicate.

FIGS. 21A-21D—Growth curves performed with MRSA strain USA 300 upon incubation with (FIG. 21A) C59, (FIG. 21B) C59Na, (FIG. 21C) 4-76, and (FIG. 21D) vancomycin hydrochloride for 18-h. Absorbance readings were registered at 10-m intervals.

FIGS. 22A-22C—Growth curves performed with MRSA strain SA LL06 upon incubation with (FIG. 22A) C58, (FIG. 22B) C59, and (FIG. 22C) vancomycin hydrochloride for 18-h. Absorbance readings were registered at 10-m intervals.

FIGS. 23A-23C—Growth curves performed with S. epidermidis strain M0881 upon incubation with (FIG. 23A) C59, (FIG. 23B) 4-76, and (FIG. 23C) vancomycin hydrochloride for 18-h. Absorbance readings were registered at 10-m intervals.

FIG. 24—Hemolytic activity of lead antimicrobial candidates after a 1-h incubation with erythrocytes.

FIGS. 25A & 25B—Toxicity of lead antimicrobials compared with FDA-approved membrane active agents, Gramicidin D and Amphotericin B, against mammalian cell lines (FIG. 25A) 16HBE human bronchial epithelial and (FIG. 25B) human dermal fibroblast after a 24-h incubation determined using an alamarBlue® Cell Viability Assay.

FIG. 26—Resistance acquisition during serial passaging in the presence of sub-MIC levels of clindamycin, C59, or vancomycin.

FIG. 27A-27C—Growth curves performed with MRSA strain USA 300 upon incubation with (FIG. 27A) 4-76, and 4-76 enantiomers (FIG. 27B) PK-1, and (FIG. 27C) PK-2 for 18-h. Absorbance readings were registered at 10-m intervals.

### DESCRIPTION OF ILLUSTRATIVE EMBODIMENTS

In some aspects, the present disclosure provides biaryl compounds which may be used in the treatment of bacterial infections or in the treatment of cancer. In some embodiments, these compounds are useful in the treatment of bacterial infections wherein the bacterial is resistant to one or more common antibiotic agents. In some embodiments, these compounds may be used in the treatment of cancer such as lung cancer.

### I. COMPOUNDS AND FORMULATIONS THEREOF

A. Compounds

In one aspect, the present disclosure provides compounds of the formula:

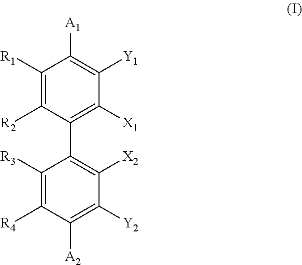

wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
   Rₐ is a monovalent amino protecting group or is taken together with R_{b} and form a divalent amino protecting group; and
   R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
   R_{c} is a hydroxy protecting group;
Y₁ is halo;
Y₂ is hydrogen or halo;
A₁ and A₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₁ and R₂ are taken together and are alkenediyl_{(C≤8)} or substituted alkenediyl_{(C≤8)}; and
R₃ and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₃ and R₄ are taken together and are alkenediyl_{(C≤8)} or substituted alkenediyl_{(C≤8)}; or a compound of the formula:

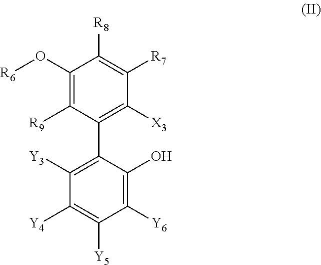

wherein:
X₃ is —OR_{A} or —NRₑR_{f}, wherein:
   R_{d} is hydrogen, alkyl_{(C≤8)}, cycloalkyl_{(C≤8)}, acyl_{(C≤8)}, or a substituted version of any of these groups; or a hydroxy protecting group;
   Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};

Additionally, the compounds provided by the present disclosure are shown, for example, above in the summary section and in the examples and claims below. They may be made using the methods outlined in the Examples section. The biaryl compounds can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The biaryl compounds of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent the biaryl compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

The biaryl compounds of the present disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the biaryl compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The biaryl compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the biaryl compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the biaryl compounds provided herein are within the scope of the disclosure. It will also be appreciated by those skilled in organic chemistry that many organic compounds can exist in more than one crystalline form. For example, crystalline form may vary from solvate to solvate. Thus, all crystalline forms of the biaryl compounds or the pharmaceutically acceptable solvates thereof are within the scope of the present disclosure.

B. Formulations

In some embodiments of the present disclosure, the compounds are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the biaryl compounds) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. BACTERIAL INFECTIONS

In some aspects of the present disclosure, the compounds disclosed herein may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodborne illness, typhoid fever, tuberculosis, pneumonia, syphilis, and leprosy.

Additionally, different bacteria have a wide range of interactions with body and those interactions can modulate ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlamydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire's disease, tuberculosis, leprosy, and brucellosis.

The compounds described herein may be used in the treatment of bacterial infections, including those caused by Staphyloccoccus aureus. S. aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains.

Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). The compounds described herein may be used in the treatment of MRSA bacterial strains.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

### A. Gram-Positive Bacteria

In some aspects of the present disclosure, the compounds disclosed herein may be used to treat a bacterial infection by a gram-positive bacteria. Gram-positive bacteria contain a thick peptidoglycan layer within the cell wall which prevents the bacteria from releasing the stain when dyed with crystal violet. Without being bound by theory, the gram-positive bacteria are often more susceptible to antibiotics. Generally, gram-positive bacteria, in addition to the thick peptidoglycan layer, also comprise a lipid monolayer and contain teichoic acids which react with lipids to form lipoteichoic acids that can act as a chelating agent. Additionally, in gram-positive bacteria, the peptidoglycan layer is outer surface of the bacteria. Many gram-positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

### B. Gram-Indeterminate Bacteria or Acid Fast Bacteria

In some aspects of the present disclosure, the compounds disclosed herein may be used to treat a bacterial infection by a gram-indeterminate bacteria. These bacteria may also be described as acid fast bacteria. Gram-indeterminate bacteria do not full stain or partially stain when exposed to crystal violet. Without being bound by theory, a gram-indeterminate bacteria may exhibit some of the properties of the gram-positive and gram-negative bacteria. A non-limiting example of a gram-indeterminate bacteria include Mycobacterium tuberculosis or Mycobacterium leprae.

### III. HYPERPROLIFERATIVE DISEASES

### A. Cancer and Other Hyperproliferative Disease

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the diaryl compounds may be used to lead to decreased cell counts and as such can potentially be used to treat a variety of types of cancer lines. In some aspects, it is anticipated that the biaryl compounds of the present disclosure may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### IV. THERAPIES

A. Pharmaceutical Formulations and Routes of Administration

Where clinical applications are contemplated, it will be necessary to prepare pharmaceutical compositions in a form appropriate for the intended application. In some embodiments, such formulation with the compounds of the present disclosure is contemplated. Generally, this will entail preparing compositions that are essentially free of pyrogens, as well as other impurities that could be harmful to humans or animals.

One will generally desire to employ appropriate salts and buffers to render delivery the compound stable and allow for uptake by target cells. Buffers also will be employed when the compounds are introduced into a patient. Aqueous compositions of the present disclosure comprise an effective amount of the compounds to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compounds of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the biaryl compounds of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate.

The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Methods of Treatment

In particular, the compositions that may be used in treating microbial infections and cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (complete blood count—CBC), or cancer cell growth or proliferation. In some embodiments, amounts of the biaryl compounds used to inhibit bacterial growth or induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the biaryl compounds may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

C. Combination Therapies

It is envisioned that the biaryl compounds may be used in combination therapies with an additional antimicrobial agent such as an antibiotic or a compound which mitigates one or more of the side effects experienced by the patient.

Furthermore, it is very common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the biaryl compounds may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Agents or factors suitable for use in a combined therapy with agents according to the present disclosure against an infectious disease include antibiotics such as penicillins, cephalosporins, carbapenems, macrolides, aminoglycosides, quinolones (including fluoroquinolones), sulfonamides and tetracylcines. Other combinations are contemplated. The following is a general discussion of antibiotic, antiviral, and cancer therapies that may be used combination with the compounds of the present disclosure.

1. Antibiotics

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930's. Since then, many different antibiotics have been developed and widely prescribed. In 2010, on average, 4 in 5 Americans are prescribed antibiotics annually. Given the prevalence of antibiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

2. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

3. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

4. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

5. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurrence of the tumor. Additionally, the compounds of the present disclosure can also be used in a neoadjuvant setting.

It also should be pointed out that any of the foregoing therapies may prove useful by themselves in treating cancer.

### V. SYNTHETIC METHODS

In some aspects, the compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the biaryl compounds.

B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanate” means —N═C═O; “azido” means —N₃; in a monovalent context “phosphate” means OP(O)(OH)₂ or a deprotonated form thereof; in a divalent context “phosphate” means —OP(O)(OH)O— or a deprotonated form thereof; “mercapto” means —SH; and “thio” means=S; “sulfato” means —SO₃H, “sulfamido” means —S(O)₂NH₂, “sulfonyl” means —S(O)₂—; and “sulfinyl” means —S(O)—.

In the context of chemical formulas, the symbol “—” means a single bond, “═” means a double bond, and “≡” means triple bond. The symbol “— — — —” represents an optional bond, which if present is either single or double. The symbol “ [symbol] ” represents a single bond or a double bond. Thus, for example, the formula

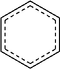

includes

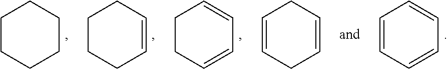

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

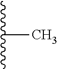

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper. When a group “R” is depicted as a “floating group” on a ring system, for example, in the formula:

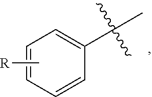

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

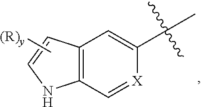

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≤n)” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. For example, “alkoxy_{(C≤10)}” designates those alkoxy groups having from 1 to 10 carbon atoms. (Cn−n′) defines both the minimum (n) and maximum number (n′) of carbon atoms in the group. Similarly, “alkyl_{(C2-10)}” designates those alkyl groups having from 2 to 10 carbon atoms.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂—, are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forms part of one or more non-aromatic ring structures, a cyclo or cyclic structure, no carbon-carbon double or triple bonds, and no atoms other than carbon and hydrogen. Non-limiting examples of cycloalkyl groups include: —CH(CH₂)₂ (cyclopropyl), cyclobutyl, cyclopentyl, or cyclohexyl. The term “cycloalkanediyl” when used without the “substituted” modifier refers to a divalent saturated aliphatic group with one or two carbon atom as the point(s) of attachment, said carbon atom(s) forms part of one or more non-aromatic ring structures, a cyclo or cyclic structure, no carbon-carbon double or triple bonds, and no atoms other than carbon and hydrogen.

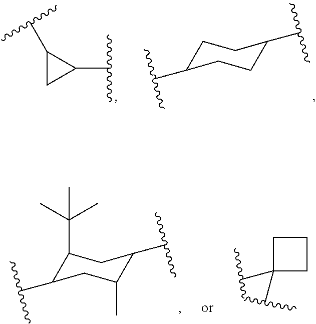

are non-limiting examples of cycloalkanediyl groups. A “cycloalkane” refers to the compound H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted cycloalkyl groups: —C(OH)(CH₂)₂,

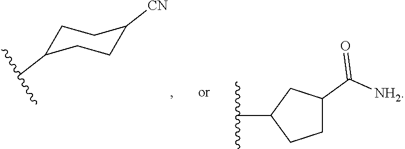 [SMILES: *C1CCC(C#N)CC1 ; *C1CCC(C(N)=O)C1]

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

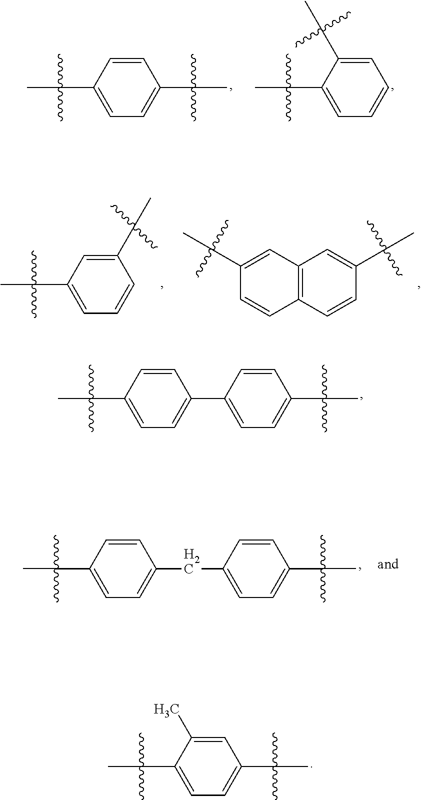

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “acyl” when used without the “substituted” modifier refers to the group —C(O)R, in which R is a hydrogen, alkyl, cycloalkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can each independently be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxylamino”, “cycloalkylamino”, “alkenylamino”, “cycloalkenylamino”, “alkynylamino”, “arylamino”, “aralkylamino”, “heteroarylamino”, “heterocycloalkylamino” and “alkylsulfonylamino” when used without the “substituted” modifier, refers to groups, defined as —NHR, in which R is alkoxy, cycloalkyl, alkenyl, cycloalkenyl, alkynyl, aryl, aralkyl, heteroaryl, heterocycloalkyl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH— alkanediyl-, —NH— alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), and —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O— alkanediyl-, —O— alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane or cycloalkane, as defined above, wherein at least one of the hydrogen atoms has been replaced with an alkoxy or cycloalkoxy group. When any of these terms is used with the “substituted” modifier one or more hydrogen atom has been independently replaced by —OH, —F, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

An “amine protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth).

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are minor images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2′, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—General Methods and Materials

All reactions were carried out in oven-dried glassware under air with magnetic stirring. All phenol and Naphthol compounds were purchased from Sigma-Aldrich Co. and used without further purification. All reactions were monitored by thin-layer chromatography (TLC) with E. Merck silica gel 60 F254 pre-coated plates (0.25 mm). Silica gel (particle size 0.032-0.063 mm) purchased from SiliCycle was used for flash chromatography. Proton (¹H) and carbon (¹³C) NMR spectra were recorded on a Bruker AV-400 (or a Bruker DRX-600) spectrometer operating at 400 MHz (or 600 MHz) for proton and 100 MHz (or 151 MHz) for carbon nuclei using CDCl₃ [or (CD₃)₂CO] as solvent, respectively. Chemical shifts are expressed as parts per million (δ, ppm) and are referenced to 7.26 (CDCl₃) or 2.05 (CD3)₂C0 for ¹H NMR and 77.00 (CDCl₃) or 206.26 (CD3)₂C0 for ¹³C NMR. Proton signal data uses the following abbreviations: s=singlet, d=doublet, t=triplet, q=quartet, m=multiplet and J=coupling constant. High Resolution Mass Spectrometry was performed on a Shimadzu LCMS-IT-TOF under the conditions of electrospray ionization (ESI) in both positive and negative mode.

X-ray Diffraction Experiment data were measured at 100(2) K on a SMART APEX II CCD area detector system equipped with a Oxford Cryosystems 700 series cooler, a graphite monochromator, and a Mo Kα fine-focus sealed tube (λ=0.71073 Å). Intensity data were processed using the Saint Plus program. All the calculations for the structure determination were carried out using the SHELXTL package (version 6.14). Initial atomic positions were located by using XT, and the structures of the compounds were refined by the least-squares method using XL. Absorption corrections were applied by using SADABS. Hydrogen atoms were placed at calculated positions and refined riding on the corresponding carbons.

### Synthesis of Quinone Monketals (Zhang et al., 2013 and Dohi et al., 2011)

### 4,4-dimethoxy-3,5-dimethylcyclohexa-2,5-dienone (1a)

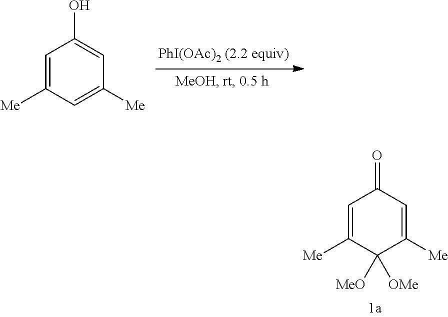 [SMILES: Cc1cc(C)cc(O)c1 ; COC1(OC)C(C)=CC(=O)C=C1C]

To a solution of 3,5-dimethylphenol (1.22 g, 10 mmol, 1.0 equiv) in distilled MeOH (20 mL) was added phenyliodoso diacetate (PIDA) (7.08 g, 22 mmol, 2.2 equiv) and the mixture was stirred at room temperature, and monitored by TLC analysis, which indicated the reaction was complete in 30 min. The reaction was quenched with sat. NaHCO₃ (50 mL) and then extracted with EtOAc (3×50 mL). The combined organic layers were washed with brine (50 mL), dried with anhydrous Na₂SO₄ and the solvent was removed in vacuo to give the crude product which was purified by flash column chromatography (SiO₂, 20:1 to 10:1, n-Hexane:EtOAc) to give the desired product 1a (1.10 g, 60% yield) as a yellow solid, R_{f}=0.50 (4:1, n-Hexane:EtOAc). ¹H NMR (400 MHz, CDCl₃): δ 6.27 (s, 2H), 3.02 (s, 6H), 1.89 (s, 6H).

### 2,5-dichloro-4,4-dimethoxycyclohexa-2,5-dien-1-one (1b)

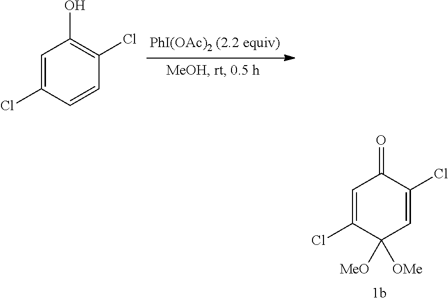 [SMILES: Oc1cc(Cl)ccc1Cl ; COC1(OC)C=C(Cl)C(=O)C=C1Cl]

The procedure of preparation was used as above. The crude product which was purified by flash column chromatography (SiO₂, 10:1 to 5:1, n-Hexane:EtOAc) to produce the desired product 1b as a light yellow solid, 40% yield, R_{f} 0.50 (5:1, n-Hexane:EtOAc). ¹H NMR (600 MHz, CDCl₃): 6.98 (s, 1H), 6.67 (s, 1H), 3.32 (s, 6H); ¹³C NMR (151 MHz, CDCl₃): δ 176.1, 153.3, 139.6, 134.9, 129.8, 96.2, 51.6.

### 4,4-dimethoxy-3-methylnaphthalen-1(4H)-one (1c)

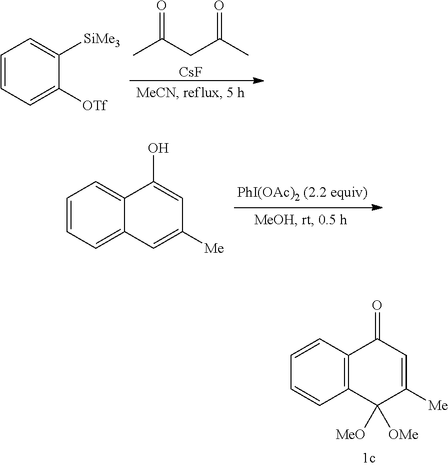

The procedure of preparation was used as above (Okuma et al., 2012). The crude product which was purified by flash column chromatography (SiO₂, 15:1 to 10:1, n-Hexane:EtOAc) to produce the desired product as a light yellow solid, 60% yield, R_{f} 0.40 (5:1, n-Hexane:EtOAc). ¹H NMR (400 MHz, CDCl₃): δ 8.05 (d, J=8.0 Hz, 1H), 7.71 (d, J=7.6 Hz, 1H), 7.67-7.61 (m, 1H), 7.48 (t, J=7.6 Hz, 1H), 6.53 (s, 1H), 2.89 (s, 6H), 2.02 (s, 3H); ¹³C NMR (100 MHz, CDCl₃): δ 183.3, 155.7, 139.3, 133.4, 132.9, 132.5, 129.3, 126.6, 125.9, 97.9, 51.2, 51.1, 16.5.

### N-(4,4-dimethoxy-3,5-dimethylcyclohexa-2,5-dien-1-ylidene)methanesulfonamide (6a)

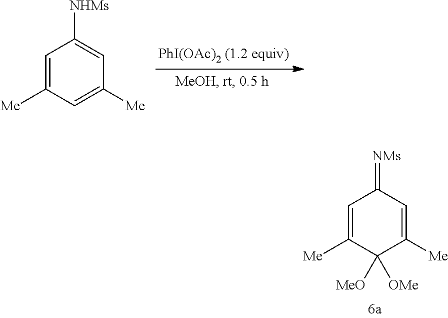

To a solution of N-(3,5-dimethylphenyl)methanesulfonamide (1.99 g, 10 mmol, 1.0 equiv) in distilled MeOH (20 mL) was added phenyliodoso diacetate (PIDA) (3.94 g, 12 mmol, 1.2 equiv) and the mixture was stirred at room temperature, and monitored by TLC analysis, which indicated the reaction was complete in 30 min. The reaction was quenched with sat. NaHCO₃ (50 mL) and then extracted with EtOAc (3×50 mL). The combined organic layers were washed with brine (50 mL), dried with anhydrous Na₂SO₄ and the solvent was removed in vacuo to give the crude product which was purified by flash column chromatography (SiO₂, 5:1 to 3:1, n-Hexane:EtOAc) to give the desired product 6a (2.08 g, 80% yield) as a yellow solid, R_{f} 0.30 (4:1, n-Hexane:EtOAc). NMR (400 MHz, CDCl₃): δ 7.37 (s, 1H), 6.35 (s, 1H), 3.14 (s, 3H), 3.00 (s, 6H), 1.97 (s, 3H), 1.94 (s, 3H); ¹³C NMR (100 MHz, CDCl₃): δ 163.5, 156.7, 154.7, 131.5, 125.2, 97.3, 50.9, 43.0, 17.1, 16.6.

### N-(4,4-dimethoxy-3,5-dimethylcyclohexa-2,5-dien-1-ylidene)-4-methylbenzenesulfonamide (6b)

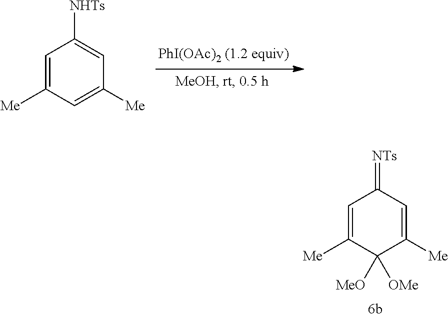

The procedure of preparation was used as 6a. The crude product which was purified by flash column chromatography (SiO₂, 5:1 to 3:1, n-Hexane:EtOAc) to produce the desired product as a light yellow solid, 60% yield, R_{f} 0.30 (4:1, n-Hexane:EtOAc). ¹H NMR (400 MHz, CDCl₃): δ 7.87 (d, J=5.6 Hz, 2H), 7.63 (s, 1H), 7.33 (d, J=5.2 Hz, 2H), 6.37 (s, 1H), 3.03 (s, 6H), 2.44 (s, 3H), 2.03 (s, 3H), 1.93 (s, 3H); ¹³C NMR (100 MHz, CDCl₃): δ 163.3, 156.8, 154.4, 143.7, 138.4, 132.0, 129.5, 127.1, 125.3, 97.5, 50.9, 21.6, 17.3, 16.6.

### N-(3,4,4,5-tetramethoxycyclohexa-2,5-dien-1-ylidene)methanesulfonamide (6c)

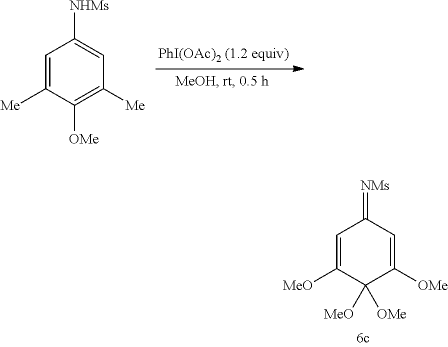

The procedure of preparation was used as 6a. The crude product which was purified by flash column chromatography (SiO₂, 3:1 to 1:1, n-Hexane:EtOAc) to produce the desired product as a light yellow solid, 72% yield, R_{f} 0.30 (1:1, n-Hexane:EtOAc). ¹H NMR (600 MHz, CDCl₃): δ 6.71 (s, 1H), 5.64 (s, 1H), 3.89 (s, 3H), 3.84 (s, 3H), 3.26 (s, 6H), 3.13 (s, 3H); ¹³C NMR (151 MHz, CDCl₃): δ 166.8, 166.7, 165.3, 103.8, 98.1, 94.8, 56.8, 56.5, 52.3, 43.1.

Optimization of the Reaction Conditions for the Conversion of 1a+2a→5a (See Table 1)

Table 1 below shows the further detailed optimization study referenced in Example 1.

TABLE 1

| Entry^{a} | Acid | Solvent | Temp. (° C.) | Time (h) | Yield^{b} (%) |
|---|---|---|---|---|---|
| 1 | TfOH | CF₃CH₂OH | 25 | 16 | <5 |
| 2 | MsOH | CF₃CH₂OH | 25 | 16 | <5 |
| 3 | HCl | CF₃CH₂OH | 25 | 16 | 48 |
| 4 | TsOH•H₂O | CF₃CH₂OH | 25 | 16 | 52 |
| 5 | TFA | CF₃CH₂OH | 25 | 16 | 59 |
| 6 | TFA | CF₃CH₂OH | 50 | 16 | 65 |
| 7 | (PhO)₂PO₂H | CF₃CH₂OH | 25 | 16 | 85 |
| 8^{c} | (PhO)₂PO₂H | CF₃CH₂OH | 25 | 18 | 75 |
| 9^{d} | (PhO)₂PO₂H | CF₃CH₂OH | 25 | 18 | 65 |
| 10^{e} | (PhO)₂PO₂H | CF₃CH₂OH | 25 | 24 | 83 |
| 11^{f} | (PhO)₂PO₂H | CF₃CH₂OH | 25 | 24 | 78 |
| 12 | (PhO)₂PO₂H | CH₂Cl₂ | 25 | 16 | N.R.^{g} |
| 13 | (PhO)₂PO₂H | CH₃CN | 25 | 30 | 62 |
| 14 | (PhO)₂PO₂H | CH₂Cl₂ | reflux | 16 | 42 |
| 15 | (PhO)₂PO₂H | CHCl₃ | 50 | 16 | 35 |
| 16 | (PhO)₂PO₂H | THF | 50 | 30 | N.R. |
| 17 | (PhO)₂PO₂H | EtOH | 50 | 30 | <5 |
| 18 | (PhO)₂PO₂H | Toluene | 50 | 30 | 80 |
| 19 | TFA | CH₂Cl₂ | 25 | 24 | N.R. |
| 20 | TFA | CH₂Cl₂ | reflux | 24 | 60 |
| 21 | TFA | DMF | 25 | 16 | N.R. |
| 22 | TFA | DMF | 100 | 16 | N.P.^{h} |
| 23 | TFA | DMSO | 25 | 16 | N.R. |
| 24 | TFA | DMSO | 100 | 16 | N.P. |
| 25 | TFA | Acetone | 25 | 16 | N.R. |
| 26 | TFA | Acetone | 50 | 16 | N.P. |
| 27 | TFA | Toluene | 25 | 16 | N.R. |
| 28 | TFA | Toluene | 50 | 18 | 64 |
| 29 | TFA | Toluene | 100 | 16 | 84 |

^{a}1a (0.3 mmol), 2a (2.0 equiv), 20 mol % of acid and 3 mL solvent were employed;
^{b}Isolated yield;
^{c}1.5 equiv of 2a was used;
^{d}1.2 equiv of 2a was used;
^{e}10 mol % of acid was used;
^{f}5 mol % of acid was used;
^{g}N.R. = No Reaction;
^{h}N.P. = No Target Product.

### General Procedure for the Synthesis of Biaryls

Procedure A:

To a stirred solution of 1a (55 mg, 0.30 mmol) and 2a (88 mg, 0.60 mmol) in toluene (3 mL), TFA (6.9 mg, 4.6 uL, 0.06 mmol) was added in one portion at room temperature, and it was stirred at 100° C. for 16 hours. Solvent was removed under reduced pressure and the crude residue was purified by column chromatography on silica-gel (hexane/ethyl acetate=20:1 to 5:1) to give pure product 5a (73.9 mg, 84% yield).

Procedure B:

To a stirred solution of 1a (36.5 mg, 0.20 mmol) and 2a (58 mg, 0.40 mmol) in CF₃CH₂OH (2 mL), (PhO)₂PO₂H (10 mg, 0.04 mmol) was added in one portion at room temperature, and it was stirred at room temperature for 16 hours. Solvent was removed under reduced pressure and the crude residue was purified by column chromatography on silica-gel (hexane/ethyl acetate=20:1 to 5:1) to give pure product 5a (50.2 mg, 85% yield).

### Example 2—Compound Characterization

### 1-(6-hydroxy-3-methoxy-2,4-dimethylphenyl)naphthalen-2-ol (5a)

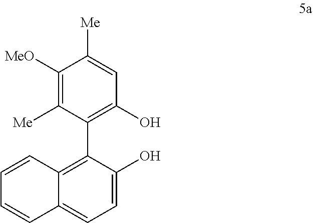 [SMILES: COc1c(C)cc(O)c(-c2c(O)ccc3ccccc23)c1C]

5a, white solid, m.p. 190-191° C.; R_{f} 0.50 (3:1, n-Hexane:EtOAc); ¹H NMR (400 MHz, Acetone-d⁶): δ 7.84-7.80 (m, 3H), 7.31-7.21 (m, 4H), 7.13 (s, 1H), 6.70 (s, 1H), 3.69 (s, 3H), 2.30 (s, 3H), 1.84 (s, 3H); ¹³C NMR (100 MHz. Acetone-d⁶): δ 153.5, 152.4, 151.3, 134.9, 132.5, 131.7, 130.0, 129.9, 128.8, 127.0, 125.1, 123.6, 120.9, 119.3, 116.8, 116.0, 60.1, 16.4, 13.4; HRMS (ESI): Exact mass calcd. for C₁₉H₁₈O₃ [M-H]⁻: 293.1183. Found: 293.1175.

### 3-bromo-1-(6-hydroxy-3-methoxy-2,4-dimethylphenyl)naphthalen-2-ol (5b)

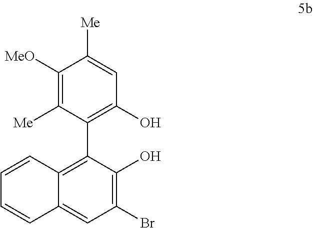 [SMILES: COc1c(C)cc(O)c(-c2c(O)c(Br)cc3ccccc23)c1C]

For 0.2 mmol scale, the standard procedure A was followed to provide 5b (48.5 mg, 65% yield); the standard procedure B was followed to provide 5b (8.9 mg, 12% yield).

5b, white solid, m.p. 208-210° C.; R_{f} 0.50 (3:1, n-Hexane:EtOAc); ¹H NMR (400 MHz, Acetone-d⁶): δ 8.20 (s, 1H), 8.04 (br s, 1H), 7.84-7.81 (m, 1H), 7.53 (br s, 1H), 7.35-7.32 (m, 2H), 7.20-7.17 (m, 1H), 6.72 (s, 1H), 3.69 (s, 3H), 2.31 (s, 3H), 1.83 (s, 3H); ¹³C NMR (100 MHz, Acetone-d⁶): δ 152.5, 151.3, 149.8, 133.9, 132.6, 132.5, 130.4, 128.0, 127.4, 125.2, 124.8, 119.9, 119.0, 116.32, 116.27, 113.6, 60.1, 16.4, 13.3; HRMS (ESI): Exact mass calcd. for C₁₉H₁₇O₃Br [M-H]⁻: 371.0288, Found: 371.0301.

### 6-bromo-1-(6-hydroxy-3-methoxy-2,4-dimethylphenyl)naphthalen-2-ol (5c)

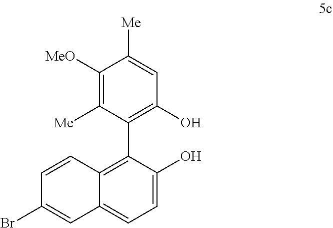 [SMILES: COc1c(C)cc(O)c(-c2c(O)ccc3cc(Br)ccc23)c1C]

For 0.2 mmol scale, the standard procedure A was followed to provide 5c (56.8 mg, 76% yield); the standard procedure B was followed to provide 5c (35.7 mg, 48% yield); For 98 mmol scale, the standard procedure A was followed to provide 5c (25.64 g, 70% yield).

5c, white solid, m.p. 217-218° C.; R_{f} 0.50 (3:1, n-Hexane:EtOAc); ¹H NMR (400 MHz, Acetone-d⁶): δ 8.05-8.03 (m, 2H), 7.80 (d, J=8.8 Hz, 1H), 7.41 (dd, J=2.0, 9.2 Hz, 1H), 7.33-7.30 (m, 2H), 7.16 (d, J=8.8 Hz, 1H), 6.70 (s, 1H), 3.69 (s, 3H), 2.30 (s, 3H), 1.84 (s, 3H); ¹³C NMR (100 MHz, Acetone-d⁶): δ 154.0, 152.4, 151.2, 133.5, 132.4, 132.0, 131.0, 130.6, 129.9, 129.2, 127.4, 120.6, 117.4, 116.7, 116.08, 116.05, 60.1, 16.4, 13.3; HRMS (ESI): Exact mass calcd. for C₁₉H₁₇O₃Br [M-H]⁻: 371.0288, Found: 371.0299.

### 7-bromo-1-(6-hydroxy-3-methoxy-2,4-dimethylphenyl)naphthalen-2-ol (5d)

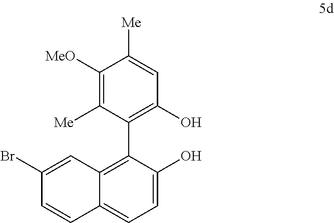 [SMILES: COc1c(C)cc(O)c(-c2c(O)ccc3ccc(Br)cc23)c1C]

For 0.2 mmol scale, the standard procedure A was followed to provide 5d (55.8 mg, 75% yield).

5d, white solid, m.p. 76-78° C.; R_{f} 0.50 (3:1, n-Hexane:EtOAc); ¹H NMR (400 MHz, Acetone-d⁶): δ 8.13 (br s, 1H), 7.84 (d, J=9.2 Hz, 1H), 7.79 (d, J=8.0 Hz, 1H), 7.39-7.36 (m, 3H), 7.31 (d, J=8.4 Hz, 1H), 6.71 (s, 1H), 3.69 (s, 3H), 2.30 (s, 3H), 1.86 (s, 3H); ¹³C NMR (100 MHz, Acetone-d⁶): δ 154.4, 152.3, 151.2, 136.2, 132.4, 132.1, 130.9, 130.0, 128.2, 126.9, 126.5, 121.0, 120.1, 119.9, 116.5, 116.1, 60.1, 16.3, 13.3; HRMS (ESI): Exact mass calcd. for C₁₉H₁₇O₃Br [M-H]⁻: 371.0288, Found: 371.0298.

### 1-(6-hydroxy-3-methoxy-2,4-dimethylphenyl)-3-methoxynaphthalen-2-ol (5e)

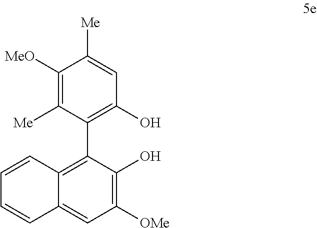 [SMILES: COc1cc2ccccc2c(-c2c(O)cc(C)c(OC)c2C)c1O]

For 0.2 mmol scale, the standard procedure B was followed to provide 5e (45.6 mg, 70% yield).

5e, white solid, m.p. 241-242° C.; R_{f} 0.50 (2:1, n-Hexane:EtOAc); ¹H NMR (400 MHz, Acetone-d⁶): δ 7.75 (d, J=8.4 Hz, 1H), 7.74 (br s, 1H), 7.33 (s, 1H), 7.28-7.17 (m, 4H), 6.68 (s, 1H), 4.02 (s, 3H), 3.68 (s, 3H), 2.30 (s, 3H), 1.84 (s, 3H); ¹³C NMR (100 MHz, Acetone-d⁶) δ: 152.0, 151.1, 149.3, 145.5, 132.0, 131.3, 130.1, 130.0, 127.7, 124.9, 124.7, 124.2, 121.7, 117.4, 115.9, 106.6, 60.2, 56.1, 16.4, 13.5; HRMS (ESI): Exact mass calcd. for C₂₀H₂₀O₄ [M-H]⁻: 323.1289, Found: 323.1294.

### 1-(6-hydroxy-3-methoxy-2,4-dimethylphenyl)-7-methoxynaphthalen-2-ol (5f)

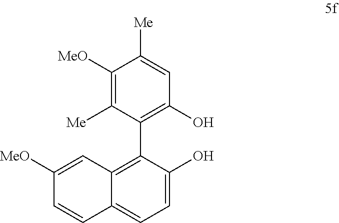 [SMILES: COc1ccc2ccc(O)c(-c3c(O)cc(C)c(OC)c3C)c2c1]

For 0.2 mmol scale, the standard procedure A was followed to provide 5f (38.8 mg, 60% yield); the standard procedure B was followed to provide 5f (35.7 mg, 55% yield).

5f, white solid, m.p. 166-167° C.; R_{f} 0.50 (2:1, n-Hexane:EtOAc); ¹H NMR (400 MHz, Acetone-d⁶): δ 7.76 (d, J=7.6 Hz, 1H), 7.74-7.71 (m, 2H), 7.09 (d, J=8.0 Hz, 1H), 7.06 (s, 1H), 6.95 (dd, J=2.4, 9.2 Hz, 1H), 6.70 (s, 1H), 6.59 (d, J=2.0 Hz, 1H), 3.69 (s, 3H), 3.64 (s, 3H), 2.30 (s, 3H), 1.86 (s, 3H); ¹³C NMR (100 MHz, Acetone-d⁶): δ 159.3, 154.1, 152.3, 151.3, 136.3, 132.4, 131.7, 130.4, 129.8, 125.2, 121.0, 116.7, 116.1, 116.0, 115.4, 104.2, 60.1, 55.2, 16.4, 13.4; HRMS (ESI): Exact mass calcd. for C₂₀H₂₀O₄ [M-H]⁻: 323.1289, Found: 323.1296.

### 1-(6-hydroxy-3-methoxy-2,4-dimethylphenyl)naphthalene-2,3-diol (5g)

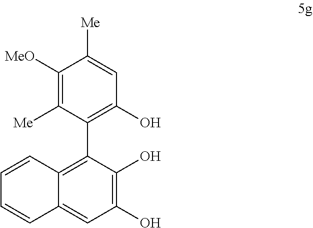 [SMILES: COc1c(C)cc(O)c(-c2c(O)c(O)cc3ccccc23)c1C]

For 0.2 mmol scale, the standard procedure A was followed to provide 5g (43.6 mg, 70% yield); the standard procedure B was followed to provide 5g (37.3 mg, 60% yield); For 1 mmol scale, the standard procedure A was followed to provide 5g (210 mg, 68% yield).

5g, white solid, m.p. 197-198° C.; R_{f} 0.40 (1:1, n-Hexane:EtOAc); ¹H NMR (400 MHz, Acetone-d⁶): δ 7.84 (br s, 3H), 7.65 (d, J=8.0 Hz, 1H), 7.26 (s, 1H), 7.25-7.20 (m, 1H), 7.13 (d, J=8.0 Hz, 1H), 6.69 (s, 1H), 3.69 (s, 3H), 2.30 (s, 3H), 1.85 (s, 3H); ¹³C NMR (100 MHz, Acetone-d⁶): δ 152.1, 151.0, 146.9, 144.8, 132.2, 131.6, 130.4, 129.4, 127.0, 124.8, 124.0, 120.9, 117.3, 115.9, 115.8, 109.9, 60.0, 16.3, 13.3; HRMS (ESI): Exact mass calcd. for C₁₉H₁₈O₄ [M-H]⁻: 309.1132, Found: 309.1144.

### Methyl 6-hydroxy-5-(6-hydroxy-3-methoxy-2,4-dimethylphenyl)-2-naphthoate (5h)

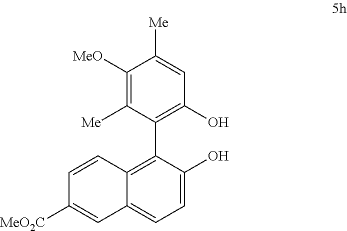 [SMILES: COC(=O)c1ccc2c(-c3c(O)cc(C)c(OC)c3C)c(O)ccc2c1]

For 0.2 mmol scale, the standard procedure A was followed to provide 5h (63.6 mg, 90% yield).

5h, white solid. m.p. 226-227° C.; R_{f} 0.35 (2:1, n-Hexane:EtOAc); ¹H NMR (400 MHz, Acetone-d⁶) δ 8.56 (s, 1H), 8.00 (d, J=8.8 Hz, 1H), 7.86 (d, J=8.8 Hz, 1H), 7.76 (br s, 2H), 7.36 (d, J=9.2 Hz, 1H), 7.30 (d, J=8.8 Hz, 1H), 6.71 (s, 1H), 3.90 (s, 3H), 3.69 (s, 3H), 2.30 (s, 3H), 1.84 (s, 3H); ¹³C NMR (100 MHz, Acetone-d⁶) δ 167.5, 155.8, 152.4, 151.2, 137.3, 132.4, 132.0, 131.8, 131.6, 128.7, 126.2, 125.4, 125.3, 120.4, 120.3, 116.10, 116.07, 60.1, 52.2, 16.4, 13.3; HRMS (ESI): Exact mass calcd. for C₂₁H₂₀O₅ [M-H]⁻: 351.1238, Found: 351.1248.

### 1-(2,5-dichloro-6-hydroxy-3-methoxyphenyl)naphthalen-2-ol (5i)

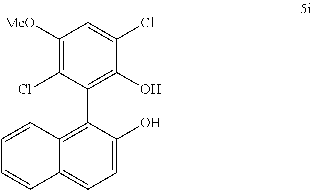 [SMILES: COc1cc(Cl)c(O)c(-c2c(O)ccc3ccccc23)c1Cl]

For 0.2 mmol scale, the standard procedure A was followed to provide 5i (10.3 mg, 15% yield); the standard procedure B was followed to provide 5i (45.6 mg, 68% yield); For 5 mmol scale, the standard procedure B was followed to provide 5i (1.01g, 60% yield).

5i, yellow oil; R_{f} 0.50 (2:1, n-Hexane:EtOAc); ¹H NMR (400 MHz, CDCl₃): δ 7.89-7.82 (m, 2H), 7.42-7.34 (m, 2H), 7.27-7.20 (m, 2H), 7.12 (s, 1H), 5.20 (br s, 2H), 3.92 (s, 3H); ¹³C NMR (100 MHz, CDCl₃): δ 151.3, 149.7, 144.8, 132.3, 131.3, 129.0, 128.3, 127.3, 123.9, 123.5, 123.1, 122.1, 118.9, 117.8, 113.7, 112.3, 56.8; HRMS (ESI): Exact mass calcd. for C₁₇H₁₂O₃Cl₂ [M-H]⁻: 333.0091, Found: 333.0091.

### 6-bromo-1-(2,5-dichloro-6-hydroxy-3-methoxyphenyl)naphthalen-2-ol (5j)

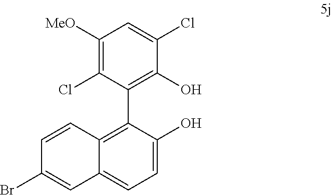 [SMILES: COc1cc(Cl)c(O)c(-c2c(O)ccc3cc(Br)ccc23)c1Cl]

For 0.2 mmol scale, the standard procedure A was followed to provide 5j (60.1 mg, 72% yield); For 40 mmol scale, the standard procedure A was followed to provide 5j (9.95g, 60% yield).

5j, yellow oil; R_{f} 0.50 (2:1, n-Hexane:EtOAc); ¹H NMR (400 MHz, CDCl₃): δ 7.98 (d, J=2.0 Hz, 1H), 7.78 (d, J=8.8 Hz, 1H), 7.43 (dd, J=2.0, 8.8 Hz, 1H), 7.27 (d, J=9.2 Hz, 1H), 7.13 (s, 1H), 7.08 (d, J=8.8 Hz, 1H), 5.31 (br s, 1H), 5.19 (br s, 1H), 3.93 (s, 3H); ¹³C NMR (100 MHz, CDCl₃): δ 151.5, 149.8, 144.7, 130.9, 130.5, 130.3, 130.2, 125.5, 123.2, 121.6, 119.0, 118.9, 117.6, 113.71, 113.68, 113.0, 56.9; HRMS (ESI): Exact mass calcd. for C₁₇H₁₁O₃BrCl₂ [M-H]⁻: 410.9196, Found: 410.9195.

### Methyl 5-(2,5-dichloro-6-hydroxy-3-methoxyphenyl)-6-hydroxy-2-naphthoate (5k)

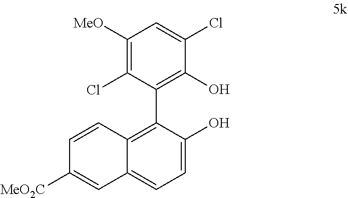 [SMILES: COC(=O)c1ccc2c(-c3c(O)c(Cl)cc(OC)c3Cl)c(O)ccc2c1]

For 0.3 mmol scale, the standard procedure A was followed to provide 5k (75 mg, 64% yield).

5k, white solid, m.p. 205-206° C.; R_{f} 0.30 (2:1, n-Hexane:EtOAc); ¹H NMR (600 MHz, Acetone-d⁶): δ 8.91 (s, 1H), 8.59 (d, J=1.2 Hz, 1H), 8.07 (d, J=9.0 Hz, 1H), 7.92 (dd, J=8.4, 1.8 Hz, 1H), 7.81 (s, 1H), 7.41 (d, J=8.4 Hz, 1H), 7.29 (s, 1H), 7.28 (d, J=8.4 Hz, 1H), 3.96 (s, 3H), 3.93 (s, 3H); ¹³C NMR (151 MHz, Acetone-d⁶): δ 166.6, 155.3, 149.3, 146.1, 136.0, 131.8, 131.0, 127.8, 125.8, 124.7, 124.6, 124.0, 122.7, 119.5, 119.2, 114.4, 113.4, 56.3, 51.4; HRMS (ESI): Exact mass calcd. for C₁₉H₁₄O₅C12 [M-H]⁻: 391.0146, Found: 391.0145.

### 4′-methoxy-3′-methyl-[1,2′-binaphthalene]-1′,2-diol (5l)

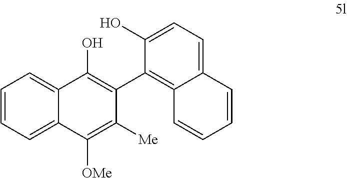 [SMILES: COc1c(C)c(-c2c(O)ccc3ccccc23)c(O)c2ccccc12]

For 0.2 mmol scale, the standard procedure A was followed to provide 5l (46 mg, 70% yield).

5l, white solid, m.p. 202-203° C.; R_{f} 0.45 (2:1, n-Hexane:EtOAc); NMR (400 MHz, CDCl₃): δ 8.27 (d, J=8.0 Hz, 1H), 8.15 (d, J=8.4 Hz, 1H), 7.93 (d, J=9.2 Hz, 1H), 7.89-7.86 (m, 1H), 7.65-7.61 (m, 1H), 7.55-7.51 (m, 1H), 7.39-7.34 (m, 3H), 7.26-7.24 (m, 1H), 5.17 (br s, 1H), 5.12 (br s, 1H), 3.94 (s, 3H), 2.03 (s, 3H); ¹³C NMR (100 MHz, CDCl₃): δ 152.2, 147.9, 146.9, 133.1, 131.2, 129.4, 129.1, 128.4, 127.5, 127.3, 126.6, 125.2, 124.0, 123.8, 123.6, 123.0, 121.7, 117.6, 112.4, 112.2, 61.6, 13.1; HRMS (ESI): Exact mass calcd. for C₂₂H₁₈O₃ [M-H]⁻: 329.1183, Found: 329.1195.

### 2-(6-hydroxy-3-methoxy-2,4-dimethylphenyl)naphthalen-1-ol (5m)

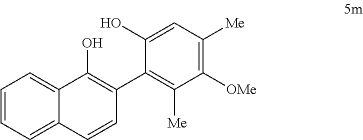 [SMILES: COc1c(C)cc(O)c(-c2ccc3ccccc3c2O)c1C]

For 0.2 mmol scale, the standard procedure A was followed to provide 5m (40.5 mg, 69% yield).

5m, white solid, m.p. 125-127° C.; R_{f} 0.50 (4:1, n-Hexane:EtOAc); ¹H NMR (400 MHz, Acetone-d⁶): δ 8.34-8.29 (m, 1H), 7.88-7.83 (m, 2H), 7.53-7.42 (m, 4H), 7.15 (d. J=8.8 Hz, 1H), 6.67 (s, 1H), 3.68 (s, 3H), 2.26 (s, 3H), 1.99 (s, 3H); ¹³C NMR (100 MHz, Acetone-d⁶): δ 152.1, 151.3, 150.8, 135.4, 132.2, 131.8, 130.4, 128.3, 126.8, 126.3, 125.6, 123.4, 123.3, 120.1, 117.8, 116.1, 60.0, 16.3, 13.8; HRMS (ESI): Exact mass calcd. for C₁₉H₁₈O₃ [M-H]⁻: 293.1183, Found: 293.1185.

### 4-(6-hydroxy-3-methoxy-2,4-dimethylphenyl)naphthalen-1-ol (5m′)

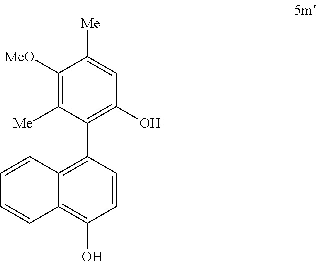 [SMILES: COc1c(C)cc(O)c(-c2ccc(O)c3ccccc23)c1C]

For 0.2 mmol scale, the standard procedure B was followed to provide 5m′ (47.2 mg, 80% yield).

5m′, white solid; m.p. 190-191° C.; R_{f} 0.50 (3:1, n-Hexane:EtOAc); ¹H NMR (400 MHz, Acetone-d⁶): δ 9.11 (s, 1H), 8.31 (d, J=8.4 Hz, 1H), 7.49-7.34 (m, 3H), 7.13 (d, J=8.0 Hz, 1H), 6.99 (d, J=8.0 Hz, 1H), 6.96 (s, 1H), 6.69 (s, 1H), 3.68 (s, 3H), 2.30 (s, 3H), 1.81 (s, 3H); ¹³C NMR (100 MHz, Acetone-d⁶): δ 153.6, 151.9, 151.0, 134.6, 131.7, 131.2, 129.3, 127.0, 126.7, 126.23, 126.18, 125.3, 123.2, 115.73, 115.69, 108.9, 60.1, 16.3, 13.8; HRMS (ESI): Exact mass calcd. for C₁₉H₁₈O₃ [M-H]⁻: 293.1183, Found: 293.1186.

### 5-methoxy-4,4′,6,6′-tetramethyl-[1,1′-biphenyl]-2,2′-diol (5n)

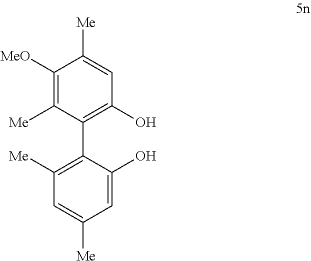 [SMILES: COc1c(C)cc(O)c(-c2c(C)cc(C)cc2O)c1C]

For 0.3 mmol scale, the standard procedure A was followed to provide 5n (44 mg, 54% yield).

5n, white solid, m.p. 128-129° C.; R_{f} 0.55 (3:1, n-Hexane:EtOAc); ¹H NMR (600 MHz, CDCl₃): δ 6.74 (s, 1H), 6.712 (s, 1H), 6.706 (s, 1H), 4.71 (br s, 1H), 4.55 (br s, 1H), 3.70 (s, 3H), 2.33 (s, 3H), 2.31 (s, 3H), 1.96 (s, 3H), 1.94 (s, 3H); ¹³C NMR (151 MHz, CDCl₃): δ 153.7, 151.0, 149.7, 140.2, 138.7, 132.9, 131.3, 123.5, 118.1, 116.7, 115.1, 113.7, 60.1, 21.3, 19.4, 16.3, 13.0; HRMS (ESI): Exact mass calcd. for C₁₇H₂₀O₃ [M-H]⁻: 271.1340, Found: 271.1339.

### 4′,5,6′-trimethoxy-4,6-dimethyl-[1,1′-biphenyl]-2,2′-diol (5o)

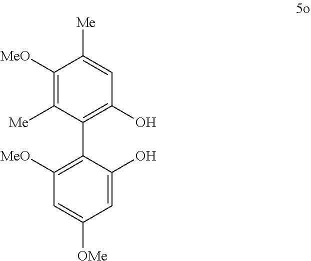 [SMILES: COc1cc(O)c(-c2c(O)cc(C)c(OC)c2C)c(OC)c1]

For 0.3 mmol scale, the standard procedure A was followed to provide 5o (59 mg, 65% yield).

5o, brown solid, m.p. 146-148° C.; R_{f} 0.30 (3:1, n-Hexane:EtOAc); NMR (600 MHz, CDCl₃): δ 6.70 (s, 1H), 6.25 (d, J=2.4 Hz, 1H), 6.18 (d, J=2.4 Hz, 1H), 5.02 (br s, 1H), 4.77 (br s, 1H), 3.82 (s, 3H), 3.701 (s, 3H), 3.695 (s, 3H), 2.29 (s, 3H), 1.97 (s, 3H); ¹³C NMR (151 MHz, CDCl₃): δ 162.1, 158.9, 155.5, 150.9, 150.3, 132.8, 132.4, 115.5, 115.0, 101.4, 93.0, 91.9, 60.0, 55.7, 55.4, 16.3, 13.1; HRMS (ESI): Exact mass calcd. for C₁₇H₂₀O₅ [M-H]⁻: 303.1238, Found: 303.1240.

### 4′,6′-dichloro-5-methoxy-4,6-dimethyl-[1,1′-biphenyl]-2,2′-diol (5p)

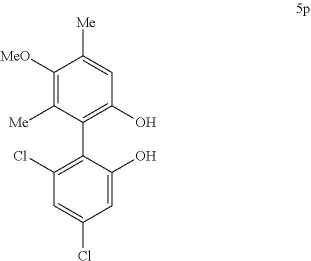 [SMILES: COc1c(C)cc(O)c(-c2c(O)cc(Cl)cc2Cl)c1C]

For 0.3 mmol scale, the standard procedure A was followed to provide 5p (71.5 mg, 76% yield).

5p, white solid, m.p. 144-146° C.; R_{f} 0.50 (3:1, n-Hexane:EtOAc); NMR (600 MHz, Acetone-d⁶): δ 8.54 (s, 1H), 7.68 (s, 1H), 7.08 (d, J=1.8 Hz, 1H), 6.97 (d, J=2.4 Hz, 1H), 6.63 (s, 1H), 3.66 (s, 3H), 2.25 (s, 3H), 1.95 (s, 3H); ¹³C NMR (151 MHz, Acetone-d⁶): δ 157.1, 150.8, 150.1, 136.0, 133.3, 131.3, 130.8, 123.1, 119.9, 119.4, 115.1, 114.5, 59.3, 15.5, 12.4; HRMS (ESI): Exact mass calcd. for C₁₅H₁₄Cl₂O₃ [M-H]⁻: 311.0247, Found: 311.0247.

### 4′,6′-dibromo-5-methoxy-4,6-dimethyl-[1,1′-biphenyl]-2,2′-diol (5q)

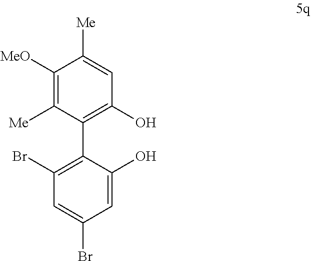 [SMILES: COc1c(C)cc(O)c(-c2c(O)cc(Br)cc2Br)c1C]

For 0.3 mmol scale, the standard procedure A was followed to provide 5q (83.2 mg, 69% yield).

5q, brown solid, m.p. 147-149° C.; R_{f} 0.50 (3:1, n-Hexane:EtOAc); NMR (600 MHz, CDCl₃): δ 7.46 (d, J=1.8 Hz, 1H), 7.17 (d, J=1.8 Hz, 1H), 6.68 (s, 1H), 5.16 (br s, 1H), 4.60 (br s, 1H), 3.70 (s, 3H), 2.30 (s, 3H), 1.95 (s, 3H); ¹³C NMR (151 MHz, CDCl₃): δ 155.2, 151.2, 149.2, 134.1, 131.5, 127.6, 125.9, 123.6, 122.0, 118.3, 117.9, 115.9, 60.2, 16.4, 13.0; HRMS (ESI): Exact mass calcd. for C₁₅H₁₄Br₂O₃ [M-H]⁻: 398.9237, Found: 398.9233.

### 1,4-bis(6-hydroxy-3-methoxy-2,4-dimethylphenyl)naphthalene-2,3-diol (5r)

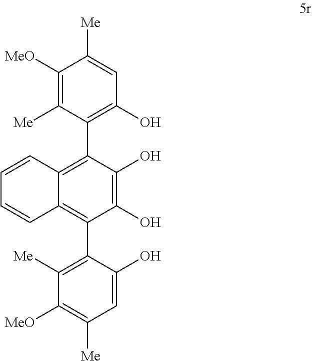 [SMILES: COc1c(C)cc(O)c(-c2c(O)c(O)c(-c3c(O)cc(C)c(OC)c3C)c3ccccc23)c1C]

For 0.2 mmol scale, the standard procedure A was followed to provide 5r (50 mg, 54% yield); For 8 mmol scale, the standard procedure A was followed to provide 5r (1.85g, 50% yield).

5r, white solid, m.p. 263-264° C.; R_{f} 0.30 (1:1, n-Hexane:EtOAc); NMR (400 MHz, CDCl₃) δ 7.31-7.24 (m, 4H), 6.77 (s, 2H), 6.02 (br s, 2H), 4.64 (br s, 2H), 3.75 (s, 6H), 2.36 (s, 6H), 1.94 (s, 6H); ¹³C NMR (100 MHz, CDCl₃) δ 151.0, 149.7, 142.4, 132.9, 131.8, 128.2, 125.3, 124.4, 117.8, 115.4, 115.0, 60.2, 16.3, 13.4; HRMS (ESI): Exact mass calcd. for C₂₈H₂₈O₆ [M-H]⁻: 459.1813, Found: 459.1816.

### N-(2-(2-hydroxynaphthalen-1-yl)-4-methoxy-3,5-dimethylphenyl)methanesulfonamide (7a)

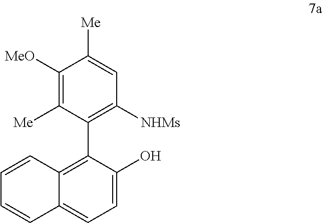

For 0.2 mmol scale, the standard procedure A was followed to provide 7a (62.5 mg, 84% yield); the standard procedure B was followed to provide 7a (68 mg, 92% yield).

7a, white solid, m.p. 187-188° C.; R_{f} 0.50 (2:1, n-Hexane:EtOAc); ¹H NMR (400 MHz, CDCl₃): δ 7.87-7.83 (m, 2H), 7.48 (s, 1H), 7.37-7.24 (m, 2H), 7.24 (d, J=8.8 Hz, 1H), 7.12-7.09 (m, 1H), 5.85 (s, 1H), 5.80 (br s, 1H), 3.76 (s, 3H), 2.66 (s, 3H), 2.40 (s, 3H), 1.87 (s, 3H); ¹³C NMR (100 MHz, CDCl₃): δ 154.9, 151.0, 132.8, 132.7, 132.5, 131.7, 130.9, 129.2, 128.6, 127.5, 124.3, 124.0, 123.1, 121.6, 117.8, 114.1, 60.0, 39.4, 16.4, 13.3; HRMS (ESI): Exact mass calcd. for C₂₀H₂₁NO₄S [M-H]⁻: 370.1119, Found: 370.1119.

### N-(2-(3-bromo-2-hydroxynaphthalen-1-yl)-4-methoxy-3,5-dimethylphenyl)methanesulfonamide (7b)

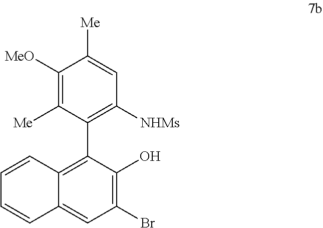

For 0.2 mmol scale, the standard procedure A was followed to provide 7b (76.1 mg, 84% yield); the standard procedure B was followed to provide 7b (53.8 mg, 60% yield).

7b, white solid, m.p. 157-159° C.; R_{f} 0.50 (2:1, n-Hexane:EtOAc); ¹H NMR (400 MHz, CDCl₃): δ 8.18 (s, 1H), 7.79-7.77 (m, 1H), 7.49 (s, 1H), 7.42-7.36 (m, 2H), 7.13-7.10 (m, 1H), 5.76 (br s, 1H), 5.74 (br s, 1H), 3.76 (s, 3H), 2.68 (s, 3H), 2.41 (s, 3H), 1.85 (s, 3H); ¹³C NMR (100 MHz, CDCl₃): δ 154.9, 147.0, 132.8, 132.6, 132.1, 132.0, 131.1, 129.8, 128.0, 127.7, 125.2, 124.9, 123.6, 121.7, 116.2, 112.4, 60.0, 39.4, 16.5, 13.3; HRMS (ESI): Exact mass calcd. for C₂₀H₂₀NO₄SBr [M-H]⁻: 448.0224, Found: 448.0234.

### N-(2-(6-bromo-2-hydroxynaphthalen-1-yl)-4-methoxy-3,5-dimethylphenyl)methanesulfonamide (7c)

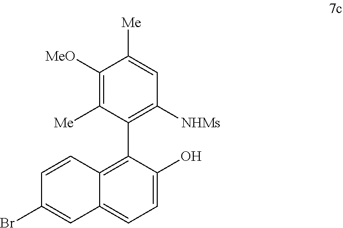

For 0.2 mmol scale, the standard procedure A was followed to provide 7c (80.1 mg, 89% yield); the standard procedure B was followed to provide 7c (57.5 mg, 64% yield); For 2 mmol scale, the standard procedure A was followed to provide 7c (785 mg, 87% yield).

7c, white solid, m.p. 199-200° C.; R_{f} 0.50 (2:1, n-Hexane:EtOAc); ¹H NMR (400 MHz, CDCl₃): δ 8.00 (d, J=1.6 Hz, 1H), 7.77 (d, J=8.4 Hz, 1H), 7.46 (s, 1H), 7.41 (dd, J=9.2, 2.0 Hz, 1H), 7.27 (d, J=9.2 Hz, 1H), 6.98 (d, J=8.8 Hz, 1H), 5.79 (s, 1H), 5.72 (br s, 1H), 3.76 (s, 3H), 2.71 (s, 3H), 2.39 (s, 3H), 1.84 (s, 3H); ¹³C NMR (100 MHz, CDCl₃): δ 154.9, 151.4, 133.3, 132.8, 131.8, 131.1, 130.7, 130.6, 130.4, 130.1, 125.0, 123.4, 121.5, 119.1, 117.8, 114.5, 60.1, 39.7, 16.5, 13.3; HRMS (ESI): Exact mass calcd. for C₂₀H₂₀NO₄SBr [M-H]⁻: 448.0224, Found: 448.0232.

### N-(2-(7-bromo-2-hydroxynaphthalen-1-yl)-4-methoxy-3,5-dimethylphenyl)methanesulfonamide (7d)

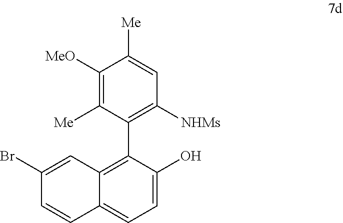

For 0.2 mmol scale, the standard procedure A was followed to provide 7d (81.1 mg, 90% yield); the standard procedure B was followed to provide 7d (51.5 mg, 57% yield).

7d, white solid, m.p. 190-191° C.; R_{f} 0.50 (2:1, n-Hexane:EtOAc); ¹H NMR (400 MHz, CDCl₃): δ 7.83 (d, J=9.2 Hz, 1H), 7.72 (d, J=8.4 Hz, 1H), 7.49 (s, 1H), 7.44 (dd, J=2.0, 8.4 Hz, 1H), 7.26 (d, J=8.4 Hz, 1H), 7.21 (d, J=1.6 Hz, 1H), 5.75 (s, 1H), 5.61 (br s, 1H), 3.77 (s, 3H), 2.75 (s, 3H), 2.41 (s, 3H), 1.86 (s, 3H); ¹³C NMR (100 MHz, CDCl₃): δ 154.9, 152.1, 133.8, 133.5, 132.9, 131.9, 131.0, 130.4, 127.7, 127.5, 125.2, 122.8, 122.2, 121.2, 118.3, 113.4, 60.1, 39.6, 16.6, 13.3; HRMS (ESI): Exact mass calcd. for C₂₀H₂₀NO₄SBr [M-H]⁻: 448.0224, Found: 448.0232.

### N-(2-(2-hydroxy-3-methoxynaphthalen-1-yl)-4-methoxy-3,5-dimethylphenyl)methanesulfonamide (7e)

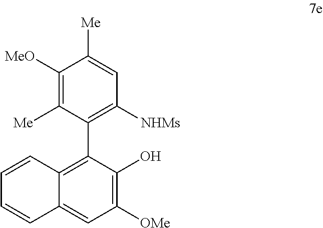

For 0.2 mmol scale, the standard procedure A was followed to provide 7e (57.1 mg, 71% yield); the standard procedure B was followed to provide 7e (67.6 mg, 84% yield).

7e, white solid, m.p. 220-222° C.; R_{f} 0.35 (2:1, n-Hexane:EtOAc); ¹H NMR (400 MHz, CDCl₃): δ 7.76 (d, J=8.0 Hz, 1H), 7.50 (s, 1H), 7.38-7.33 (m, 1H), 7.27-7.23 (m, 2H), 7.11 (d, J=8.0 Hz, 1H), 6.17 (s, 1H), 5.87 (s, 1H), 4.08 (s, 3H), 3.77 (s, 3H), 2.62 (s, 3H), 2.41 (s, 3H), 1.87 (s, 3H); ¹³C NMR (100 MHz, CDCl₃): δ 154.8, 146.8, 142.9, 132.0, 130.9, 129.2, 127.8, 127.3, 125.8, 125.3, 124.7, 123.4, 121.7, 114.8, 106.6, 60.0, 56.0, 16.4, 13.4; HRMS (ESI): Exact mass calcd. for C₂₁H₂₃NO₅S [M-H]⁻: 400.1224, Found: 400.1231.

### N-(2-(2-hydroxy-7-methoxynaphthalen-1-yl)-4-methoxy-3,5-dimethylphenyl)methanesulfonamide (70

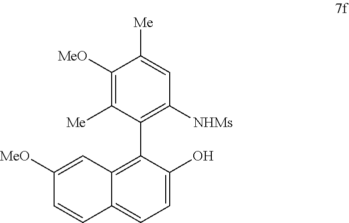

For 0.2 mmol scale, the standard procedure A was followed to provide 7f (75.6 mg, 94% yield); the standard procedure B was followed to provide 7f (46.6 mg, 58% yield).

7f, white solid. m.p. 188-190° C.; R_{f} 0.35 (2:1, n-Hexane:EtOAc); ¹H NMR (400 MHz, CDCl₃): δ 7.75 (t, J=8.8 Hz, 2H), 7.47 (s, 1H), 7.08 (d, J=8.8 Hz, 1H), 7.02 (dd, J=2.4, 8.8 Hz, 1H), 6.38 (d, J=2.4 Hz, 1H), 5.87 (s, 1H), 5.68 (br s, 1H), 3.75 (s, 3H), 3.67 (s, 3H), 2.70 (s, 3H), 2.39 (s, 3H), 1.90 (s, 3H); ¹³C NMR (100 MHz, CDCl₃): δ 159.1, 154.9, 151.6, 133.9, 132.8, 132.7, 131.7, 130.7, 130.3, 124.6, 124.4, 121.8, 115.6, 115.1, 113.3, 102.4, 60.0, 55.2, 39.4, 16.5, 13.2; HRMS (ESI): Exact mass calcd. for C₂₁H₂₃NO₅S [M-H]⁻: 400.1224, Found: 400.1231.

### N-(2-(2,3-dihydroxynaphthalen-1-yl)-4-methoxy-3,5-dimethylphenyl)methane-sulfonamide (7g)

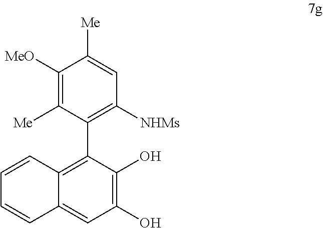

For 0.2 mmol scale, the standard procedure A was followed to provide 7g (44.8 mg, 58% yield).

7g, white solid, m.p. 87-88° C.; R_{f} 0.35 (1:1, n-Hexane:EtOAc); ¹H NMR (400 MHz, CDCl₃): δ 7.69 (d, J=8.4 Hz, 1H), 7.44 (s, 1H), 7.35-7.31 (m, 2H), 7.23-7.19 (m, 1H), 7.03 (d, J=8.0 Hz, 1H), 6.47 (br s, 1H), 5.89 (br s, 1H), 5.85 (s, 1H), 3.77 (s, 3H), 2.69 (s, 3H), 2.40 (s, 3H), 1.87 (s, 3H); ¹³C NMR (100 MHz, CDCl₃): δ 155.1, 144.3, 141.9, 132.8, 132.7, 131.4, 129.8, 127.4, 127.2, 125.2, 125.0, 124.8, 123.2, 122.1, 115.5, 111.0, 60.1, 39.7, 16.5, 13.4; HRMS (ESI): Exact mass calcd. for C₂₀H₂₁NO₅S [M-H]⁻: 386.1068, Found: 386.1081.

### Methyl 6-hydroxy-5-(3-methoxy-2,4-dimethyl-6-(methylsulfonamido)phenyl)-2-naphthoate (7h)

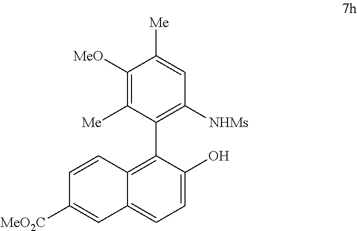

For 0.2 mmol scale, the standard procedure A was followed to provide 7h (70.6 mg, 82% yield).

7h, white solid, m.p. 178-179° C.; R_{f} 0.30 (1:1, n-Hexane:EtOAc); ¹H NMR (400 MHz, CDCl₃): δ 7.84-7.80 (m, 3H), 7.31-7.21 (m, 4H), 7.13 (s, 1H), 6.70 (s, 1H), 3.69 (s, 3H), 2.30 (s, 3H), 1.84 (s, 3H); ¹³C NMR (100 MHz, CDCl₃): δ 153.5, 152.4, 151.3, 134.9, 132.5, 131.7, 130.0, 129.9, 128.8, 127.0, 125.1, 123.6, 120.9, 119.3, 116.8, 116.0, 60.1, 16.4, 13.4; HRMS (ESI): Exact mass calcd. for C₂₂H₂₃NO₆S [M-H]⁻: 428.1173, Found: 428.1174.

### N-(2-(1-hydroxynaphthalen-2-yl)-4-methoxy-3,5-dimethylphenyl)methane-sulfonamide (7i)

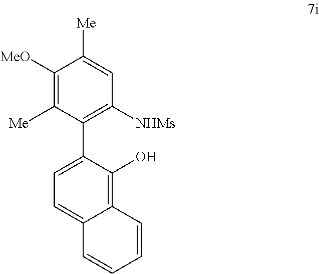

For 0.3 mmol scale, the standard procedure A was followed to provide 7i (85 mg, 76% yield); the standard procedure B was followed to provide 7i (66 mg, 59% yield);

7i, white solid, m.p. 135-137° C.; R_{f} 0.40 (1:1, n-Hexane:EtOAc); NMR (600 MHz, CDCl₃): δ 8.24 (d, J=8.4 Hz, 1H), 8.14 (d, J=8.4 Hz, 1H), 7.60-7.55 (m, 1H), 7.47-7.40 (m, 2H), 7.22 (s, 2H), 6.49 (d, J=8.4 Hz, 1H), 3.65 (s, 3H), 3.22 (s, 3H), 2.23 (s, 6H); ¹³C NMR (151 MHz, CDCl₃): δ 155.5, 152.8, 136.6, 133.0, 131.9, 128.8, 128.1, 127.7, 126.3, 125.5, 123.2, 122.5, 108.0, 59.6, 39.8, 16.2; HRMS (ESI): Exact mass calcd. for C₂₀H₂₁NO₄S [M-H]⁻: 370.1119, Found: 370.1112.

### N-(2′-hydroxy-5-methoxy-4,4′,6,6′-tetramethyl-[1,1′-biphenyl]-2-yl)methane-sulfonamide (7j)

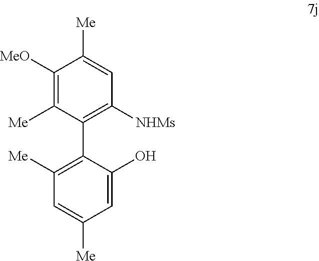

For 0.3 mmol scale, the standard procedure A was followed to provide 7j (72.2 mg, 69% yield).

7j, white solid, m.p. 216-218° C.; R_{f} 0.50 (2:1, n-Hexane:EtOAc); ¹H NMR (600 MHz, CDCl₃): δ 6.85 (s, 2H), 6.50 (s, 2H), 3.69 (s, 3H), 3.25 (s, 3H), 2.22 (s, 6H), 2.21 (s, 6H); ¹³C NMR (151 MHz, CDCl₃): δ 155.7, 153.2, 140.2, 136.3, 131.8, 129.8, 119.7, 115.8, 59.7, 39.9, 19.2, 16.4; HRMS (ESI): Exact mass calcd. for C₁₈H₂₃NO₄S [M-H]⁻: 348.1275, Found: 378.1268.

### N-(2′-hydroxy-4′,5,6′-trimethoxy-4,6-dimethyl-[1,1′-biphenyl]-2-yl)methane-sulfonamide (7k)

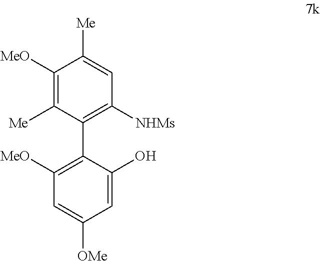

For 0.3 mmol scale, the standard procedure A was followed to provide 7k (92 mg, 80% yield).

7k, white solid, m.p. 209-210° C.; R_{f} 0.50 (1:1, n-Hexane:EtOAc); ¹H NMR (600 MHz, CDCl₃): δ 7.38 (s, 1H), 6.23 (s, 1H), 6.19 (s, 1H), 6.05 (s, 1H), 4.87 (br s, 1H), 3.83 (s, 3H), 3.74 (s, 3H), 3.69 (s, 3H), 2.76 (s, 3H), 2.34 (s, 3H), 1.98 (s, 3H); ¹³C NMR (151 MHz, CDCl₃): δ 162.1, 158.0, 155.1, 154.6, 132.9, 132.6, 132.0, 123.1, 122.6, 103.1, 93.6, 92.1, 59.9, 55.7, 55.4, 39.1, 16.4, 13.3; HRMS (ESI): Exact mass calcd. for C₁₈H₂₃NO₆S [M-H]⁻: 380.1173, Found: 380.1176.

### N-(2-(2-hydroxynaphthalen-1-yl)-4-methoxy-3,5-dimethylphenyl)-4-methylbenzene-sulfonamide (7l)

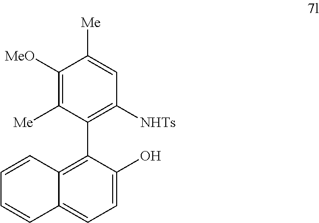

For 0.3 mmol scale, the standard procedure A was followed to provide 7l (114 mg, 85% yield); the standard procedure B was followed to provide 7l (110 mg, 82% yield).

7l, white solid, m.p. 194-195° C.; R_{f} 0.50 (2:1, n-Hexane:EtOAc); ¹H NMR (600 MHz, Acetone-d⁶): δ 8.48 (s, 1H), 7.90 (d, J=8.4 Hz, 1H), 7.85 (d, J=7.8 Hz, 1H), 7.47 (s, 1H), 7.35 (d, J=9.6 Hz, 1H), 7.33-7.27 (m, 3H), 7.16-7.12 (m, 1H), 7.00 (d, J=8.4 Hz, 2H), 6.86 (s, 1H), 6.67 (d, J=8.4 Hz, 1H), 3.70 (s, 3H), 2.36 (s, 3H), 2.30 (s, 3H), 1.74 (s, 3H); ¹³C NMR (151 MHz, Acetone-d⁶): δ 154.4, 152.1, 143.2, 137.1, 133.2, 131.8, 131.7, 130.8, 130.3, 129.3, 128.9, 128.1, 126.9, 126.8, 126.6, 123.5, 123.0, 121.2, 118.3, 114.9, 59.2, 20.6, 15.7, 12.9; HRMS (ESI): Exact mass calcd. for C₂₆H₂₅NO₄S [M-H]⁻; 446.1432, Found: 446.1430.

### N-(2-(2-hydroxy-7-methoxynaphthalen-1-yl)-4-methoxy-3,5-dimethylphenyl)-4-methylbenzenesulfonamide (7m)

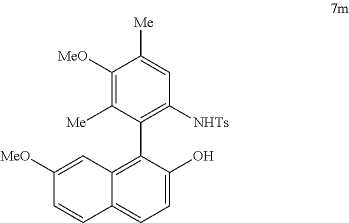

For 0.3 mmol scale, the standard procedure A was followed to provide 7m (125 mg, 87% yield).

7m, white solid, m.p. 163-165° C.; R_{f} 0.40 (3:1, n-Hexane:EtOAc); ¹H NMR (600 MHz, Acetone-d⁶): δ 8.61 (s, 1H), 7.81 (d, J=9.0 Hz, 1H), 7.74 (d, J=9.0 Hz, 1H), 7.50 (s, 1H), 7.21 (d, J=9.0 Hz, 1H), 7.17 (d, J=7.8 Hz, 2H), 6.95 (dd, J=9.0, 2.4 Hz, 1H), 6.90-6.86 (m, 3H), 5.92 (d, J=2.4 Hz, 1H), 3.71 (s, 3H), 3.47 (s, 3H), 2.37 (s, 3H), 2.25 (s, 3H), 1.79 (s, 3H); ¹³C NMR (151 MHz, Acetone-d⁶): δ 158.4, 154.8, 152.4, 143.1, 137.0, 134.8, 131.6, 131.3, 130.7, 130.0, 129.6, 129.1, 127.6, 126.7, 124.2, 122.4, 115.35, 115.33, 114.4, 102.3, 59.3, 54.1, 20.6, 15.8, 13.1; HRMS (ESI): Exact mass calcd. for C₂₇H₂₇NO₅S [M-H]⁻: 476.1537, Found: 476.1541.

### N-(2-(6-bromo-2-hydroxynaphthalen-1-yl)-4-methoxy-3,5-dimethylphenyl)-4-methylbenzenesulfonamide (7n)

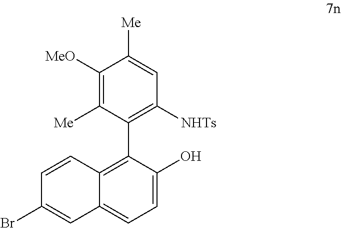

For 0.3 mmol scale, the standard procedure A was followed to provide 7n (139 mg, 88% yield).

7n, white solid, m.p. 204-206° C.; R_{f} 0.40 (3:1, n-Hexane:EtOAc); ¹H NMR (600 MHz, Acetone-d⁶): δ 8.03 (d, J=1.8 Hz, 1H), 7.87 (d, J=9.0 Hz, 1H), 7.48 (s, 1H), 7.39 (d, J=9.0 Hz, 1H), 7.22 (d, J=8.4 Hz, 2H), 7.17 (dd, J=9.0, 2.4 Hz, 1H), 6.96 (d, J=8.4 Hz, 2H), 6.53 (d, J=9.0 Hz, 1H), 3.71 (s, 3H), 2.37 (s, 3H), 2.34 (s, 3H), 1.71 (s, 3H); ¹³C NMR (151 MHz, Acetone-d⁶): δ 154.7, 152.5, 143.2, 137.3, 132.0, 131.62, 131.57, 131.1, 130.0, 129.8, 129.6, 129.4, 129.1, 126.8, 126.6, 125.8, 122.8, 119.5, 116.2, 115.5, 59.2, 20.8, 15.7, 13.0.

### methyl-6-hydroxy-5-(3-methoxy-2,4-dimethyl-6-((4-methylphenyl)sulfonamido)-phenyl)-2-naphthoate (7o)

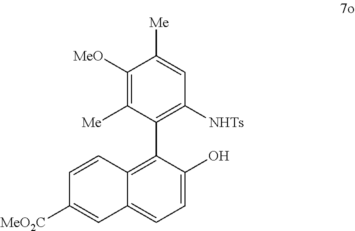

For 0.3 mmol scale, the standard procedure A was followed to provide 7o (142 mg, 94% yield).

7m, white solid, m.p. 209-210° C.; R_{f} 0.30 (2:1, n-Hexane:EtOAc); NMR (600 MHz, Acetone-d⁶): δ 8.93 (s, 1H), 8.54 (d, J=1.8 Hz, 1H), 8.06 (d, J=9.0 Hz, 1H), 7.60 (dd, J=9.0, 1.8 Hz, 1H), 7.50 (s, 1H), 7.45-7.40 (m, 1H), 7.22 (dd, J=8.4, 1.8 Hz, 2H), 6.99 (s, 1H), 6.94 (d, J=7.8 Hz, 2H), 6.65 (d, J=9.0 Hz, 1H), 3.96 (s, 3H), 3.72 (s, 3H), 2.38 (s, 3H), 2.26 (s, 3H), 1.71 (s, 3H); NMR (151 MHz, Acetone-d⁶): δ 166.6, 154.7, 154.4, 143.2, 137.3, 135.8, 131.8, 131.7, 131.6, 131.0, 130.9, 129.2, 127.8, 126.7, 126.6, 126.0, 124.6, 123.8, 122.5, 119.2, 115.5, 59.3, 51.4, 20.5, 15.7, 12.9; HRMS (ESI): Exact mass calcd. for C₂₈H₂₇NO₆S [M-H]⁻: 504.1486, Found: 504.1490.

### N-(2-(2-hydroxynaphthalen-1-yl)-3,4,5-trimethoxyphenyl)methanesulfonamide (7p)

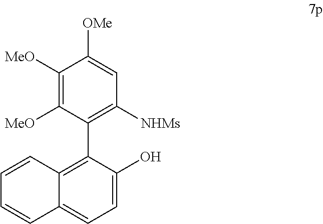

For 0.3 mmol scale, the standard procedure A was followed to provide 7p (78 mg, 64% yield).

7p, white solid, m.p. 241-242° C.; R_{f} 0.50 (1:1, n-Hexane:EtOAc); ¹H NMR (600 MHz, Acetone-d⁶): δ 8.59 (s, 1H), 7.91 (d, J=9.0 Hz, 1H), 7.88 (d, J=7.8 Hz, 1H), 7.40-7.32 (m, 3H), 7.29 (d, J=8.4 Hz, 1H), 7.22 (s, 1H), 6.61 (s, 1H), 3.96 (s, 3H), 3.87 (s, 3H), 3.52 (s, 3H), 2.72 (s, 3H); ¹³C NMR (151 MHz, Acetone-d⁶): δ 154.0, 153.0, 152.8, 140.0, 134.3, 132.4, 130.3, 129.0, 128.2, 126.7, 124.1, 123.2, 118.3, 114.4, 112.6, 101.6, 60.2, 60.1, 55.5, 38.9; HRMS (ESI): Exact mass calcd. for C₂₀H₂₁NO₆S [M-H]⁻: 402.1017, Found: 402.1019.

### N-(2-(6-bromo-2-hydroxynaphthalen-1-yl)-3,4,5-trimethoxyphenyl)methanesulfonamide (7q)

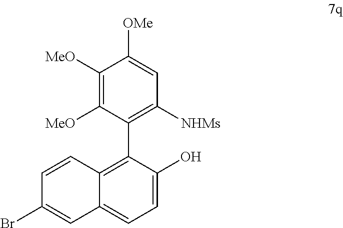

For 0.3 mmol scale, the standard procedure A was followed to provide 7q (108 mg, 74% yield).

7q, white solid, m.p. 236-237° C.; R_{f} 0.40 (1:1, n-Hexane:EtOAc); ¹H NMR (600 MHz, Acetone-d⁶): δ 8.08 (d, J=2.4 Hz, 1H), 7.89 (d, J=9.0 Hz, 1H), 7.48 (dd, J=9.0, 2.4 Hz, 1H), 7.39 (d, J=8.4 Hz, 1H), 7.26-7.22 (m, 2H), 3.96 (s, 3H), 3.87 (s, 3H), 3.52 (s, 3H), 2.75 (s, 3H); ¹³C NMR (151 MHz, Acetone-d⁶): δ 154.1, 153.5, 152.7, 140.0, 133.1, 132.6, 130.2, 130.0, 129.5, 129.4, 126.5, 119.6, 116.3, 113.9, 113.1, 101.9, 60.19, 60.15, 55.5, 39.1.

### N-(2-(2,3-dihydroxynaphthalen-1-yl)-3,4,5-trimethoxyphenyl)methanesulfonamide (7r)

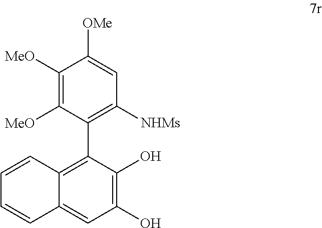

For 0.3 mmol scale, the standard procedure A was followed to provide 7r (84 mg, 67% yield).

7r, white solid, m.p. 216-217° C.; R_{f} 0.30 (1:1, n-Hexane:EtOAc); ¹H NMR (600 MHz, Acetone-d⁶): δ 7.69 (d, J=7.8 Hz, 1H), 7.35 (s, 1H), 7.30-7.25 (m, 1H), 7.24 (s, 1H), 7.23-7.17 (m, 2H), 3.96 (s, 3H), 3.87 (s, 3H), 3.53 (s, 3H), 2.74 (s, 3H); ¹³C NMR (151 MHz, Acetone-d⁶): δ 154.0, 152.8, 145.8, 144.8, 140.0, 132.4, 129.6, 128.8, 126.4, 123.9, 123.8, 123.7, 114.4, 113.2, 110.1, 101.6, 60.21, 60.17, 55.5, 38.9; HRMS (ESI): Exact mass calcd. for C₂₀H₂₁NO₇S [M-H]⁻: 418.0966, Found: 418.0970.

### Methyl 6-hydroxy-5-(2,3,4-trimethoxy-6-(methylsulfonamido)phenyl)-2-naphthoate (7s)

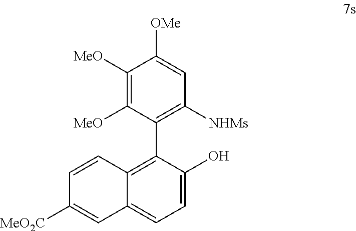

For 0.3 mmol scale, the standard procedure A was followed to provide 7s (91 mg, 66% yield).

7s, white solid, m.p. 183-184° C.; R_{f} 0.50 (1:1, n-Hexane:EtOAc); ¹H NMR (600 MHz, Acetone-d⁶): δ 8.96 (s, 1H), 8.58 (d, J=1.8 Hz, 1H), 8.08 (d, J=9.0 Hz, 1H), 7.93 (dd, J=9.0, 1.8 Hz, 1H), 7.44 (d, J=8.4 Hz, 1H), 7.38 (d, J=8.4 Hz, 1H), 7.25 (s, 1H), 6.84 (s, 1H), 3.96 (s, 3H), 3.92 (s, 3H), 3.88 (s, 3H), 3.53 (s, 3H), 2.75 (s, 3H); ¹³C NMR (151 MHz, Acetone-d⁶): δ 166.6, 155.3, 154.1, 152.7, 140.0, 136.9, 132.6, 131.8, 131.1, 128.0, 125.8, 124.9, 124.6, 119.4, 113.9, 113.1, 101.9, 60.20, 60.17, 55.5, 51.4, 39.1; HRMS (ESI): Exact mass calcd. for C₂₂H₂₃NO₈S [M+Na]⁺: 484.1037, Found: 484.1045.

### N-(2′,4′-dichloro-6′-hydroxy-4,5,6-trimethoxy-[1,1′-biphenyl]-2-yl)methane-sulfonamide (7t)

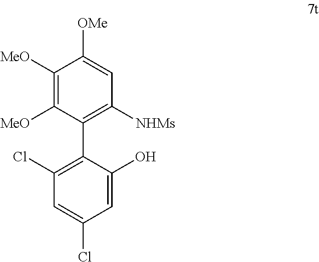

For 0.3 mmol scale, the standard procedure A was followed to provide 7t (65.8 mg, 52% yield).

7t, white solid, m.p. 195-197° C.; R_{f} 0.50 (2:1, n-Hexane:EtOAc); ¹H NMR (600 MHz, Acetone-d⁶): δ 9.11 (s, 1H), 7.22 (s, 1H), 7.16 (s, 1H), 7.10 (d, J=1.8 Hz, 1H), 7.01 (d, J=1.8 Hz, 1H), 3.91 (s, 3H), 3.83 (s, 3H), 3.70 (s, 3H), 2.92 (s, 3H); ¹³C NMR (151 MHz, Acetone-d⁶): δ 157.5, 154.2, 152.0, 139.6, 136.7, 134.3, 132.0, 120.4, 119.6, 114.8, 113.7, 102.0, 60.3, 60.1, 55.5, 39.2; HRMS (ESI): Exact mass calcd. for C₁₆H₁₇Cl₂NO₆S [M-H]⁻: 420.0081, Found: 420.0085.

### N-(2′,4′-dibromo-6′-hydroxy-4,5,6-trimethoxy-[1,1′-biphenyl]-2-yl)methane-sulfonamide (7u)

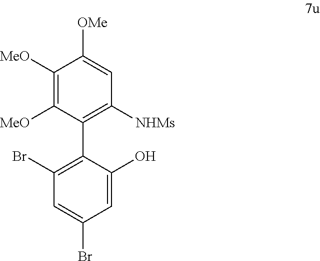

For 0.3 mmol scale, the standard procedure A was followed to provide 7u (73.5 mg, 48% yield).

7u, white solid, m.p. 209-210° C.; R_{f} 0.50 (2:1, n-Hexane:EtOAc); ¹H NMR (600 MHz, Acetone-d⁶): δ 9.10 (s, 1H), 7.40 (d, J=2.4 Hz, 1H), 7.20 (d, J=1.8 Hz, 2H), 7.17 (s, 1H), 3.91 (s, 3H), 3.83 (s, 3H), 3.71 (s, 3H), 2.95 (s, 3H); ¹³C NMR (151 MHz, Acetone-d⁶): δ 157.5, 154.2, 151.8, 139.5, 131.8, 127.3, 126.1, 122.3, 122.0, 118.2, 115.5, 101.5, 60.3, 60.1, 55.5, 39.4; HRMS (ESI): Exact mass calcd. for C₁₆H₁₇Br₂NO₆S [M-H]⁻: 507.9071, Found: 507.9070.

### Deprotection of the Mesyl (Ms) Group (Ito et al., 2013)

To a stirred solution of 7a (150 mg, 0.40 mmol) and Boc₂O (210 mg, 0.96 mmol) in CH₂Cl₂ (10 DMAP (20 mg) was added at room temperature under air, and it was stirred for 1 h. Solvent was removed under reduced pressure. The crude residue was dissolved in THF (10 mL) under Argon atmosphere and n-BuLi (1.6 M in hexane, 0.75 mL, 1.2 mmol) was added dropwise at 0° C. After stirring 20 min., Argon balloon was replaced by dry O₂ balloon and then stirred for 1 h at room temperature. Water was added to the reaction mixture and the aqueous phase was extracted with EtOAc three times. The combined extract was dried over anhydrous Na₂SO₄ and then evaporated to dryness. The crude residue was dissolved to CH₂Cl₂ (5 mL) and then TFA (0.6 mL) was added at room temperature. After reaction completion was confirmed by TLC, solvent was removed under vacuum and crude product was purified by column chromatography on SiO₂ (hexane/ethyl acetate=3:1) to give pure amine 8a (88 mg, 75% yield).

### 1-(6-amino-3-methoxy-2,4-dimethylphenyl)naphthalen-2-ol (8a)

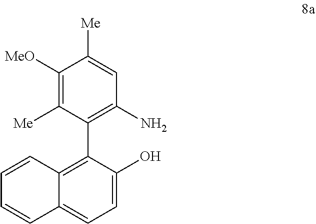 [SMILES: COc1c(C)cc(N)c(-c2c(O)ccc3ccccc23)c1C]

8a, 75% yield (3 steps), yellow amorphous; R_{f} 0.45 (2:1, n-Hexane:EtOAc); ¹H NMR (400 MHz, CDCl₃): δ 7.83 (d, J=8.8 Hz, 2H), 7.35-7.26 (m, 4H), 6.56 (s, 1H), 4.02 (br s, 3H), 3.71 (s, 3H), 2.32 (s, 6H), 1.84 (s, 3H); ¹³C NMR (100 MHz, CDCl₃): δ 151.1, 150.2, 141.1, 132.7, 132.4, 132.2, 129.9, 129.3, 128.3, 126.9, 123.9, 123.5, 117.7, 116.7, 115.80, 115.76, 60.2, 16.3, 13.1.

### Example 3—Antimicrobial Activity

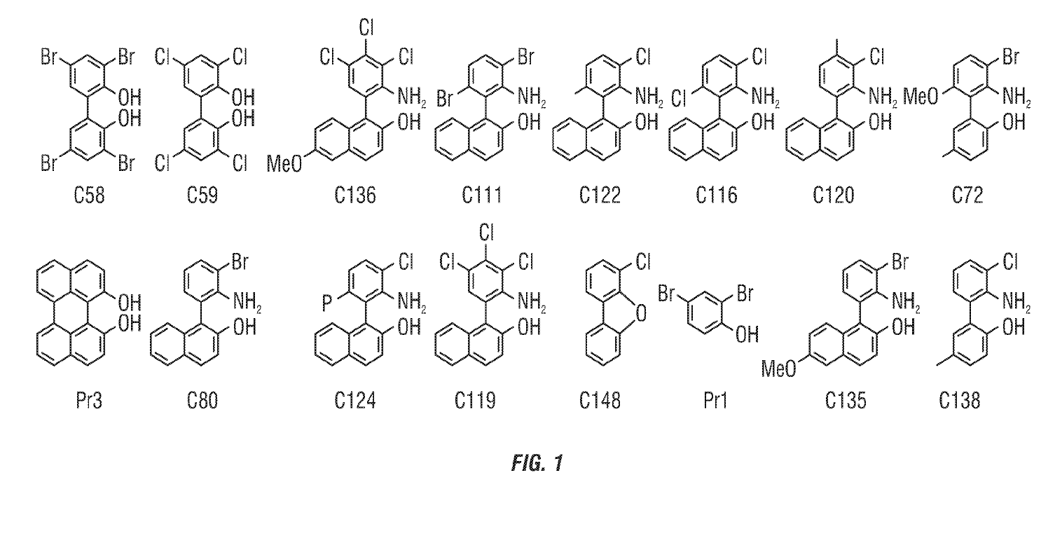

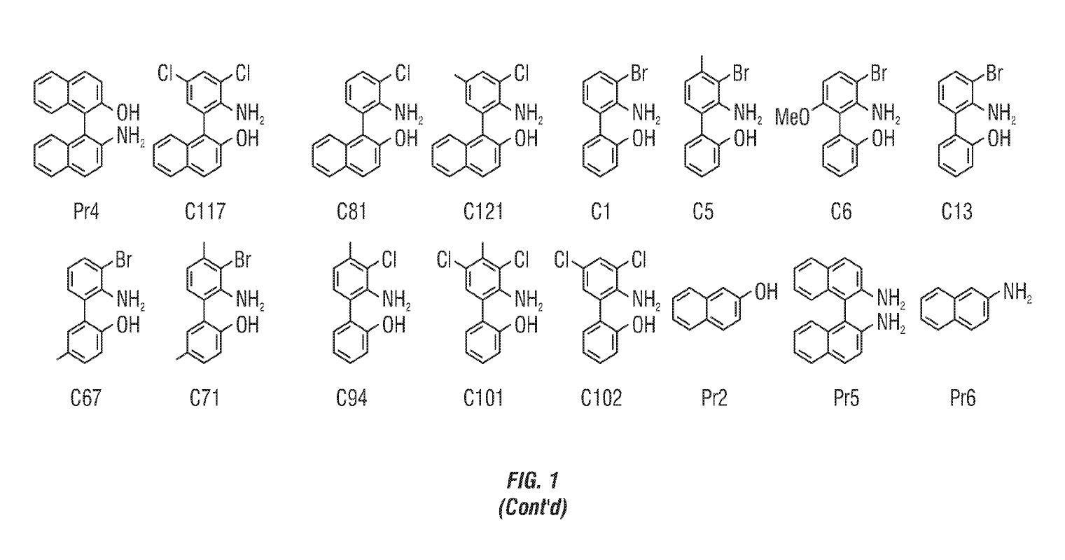

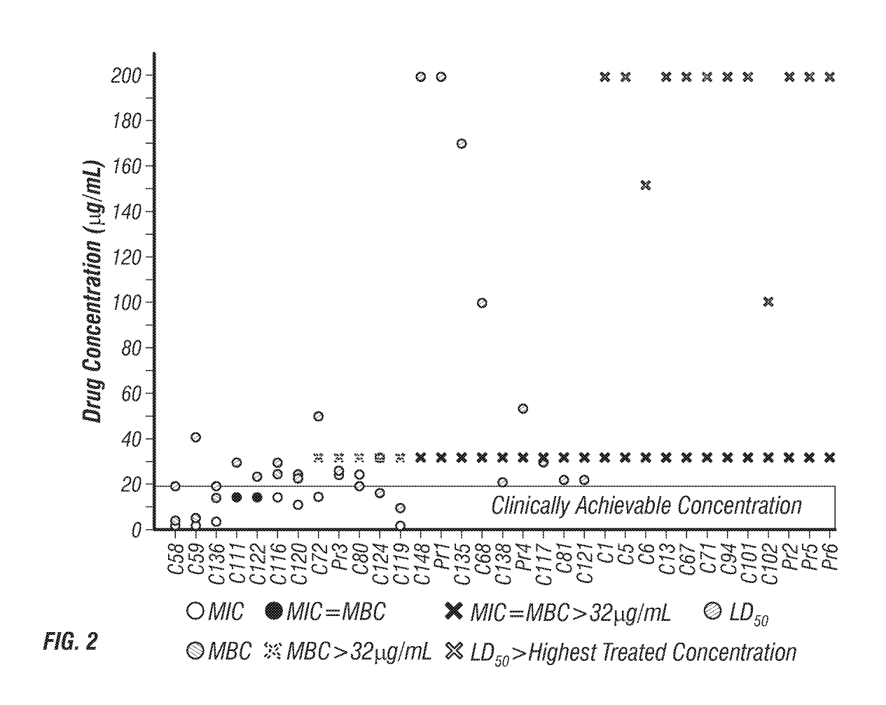

Biaryl compounds and their precursor molecules tested for antimicrobial activity against Gram-positive methicillin resistant Staphylococcus aureus (MRSA) are listed in FIG. 1. Results from in vitro screening for activity against MRSA strain USA300 (ATCC) and toxicity towards human bronchial epithelial (16HBE) cell line (as identified by LD₅₀) are reported in FIG. 2 and were used to eliminate compounds with poor antimicrobial activity or high toxicity. Compounds with a 10 fold or greater difference between bacteriostatic concentration and inhibitory concentration at median cell survival (LD₅₀), also known as therapeutic window, were chosen for further investigation. Active compounds with a smaller therapeutic window were modified using basic medicinal chemistry and structure-activity relationship principles to enhance activity and reduce toxicity. Based on these criteria, a detailed investigation was performed with C58 and C59, while C136, C111, and C122 were modified to enhance anti-microbial activity and reduce toxicity.

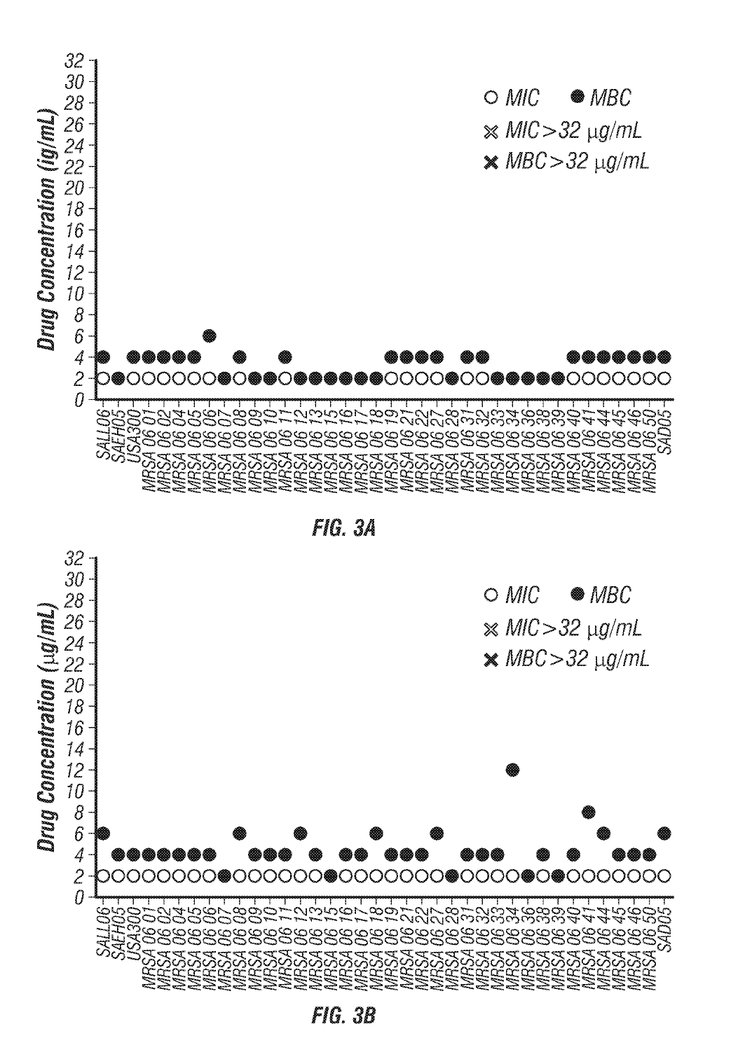

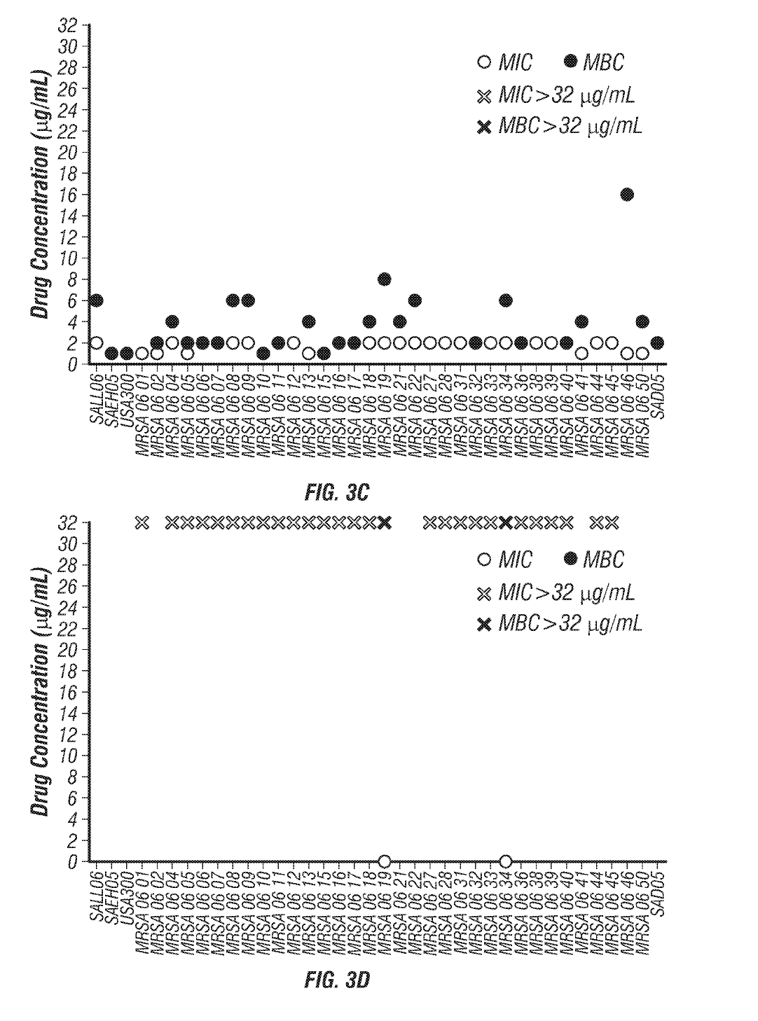

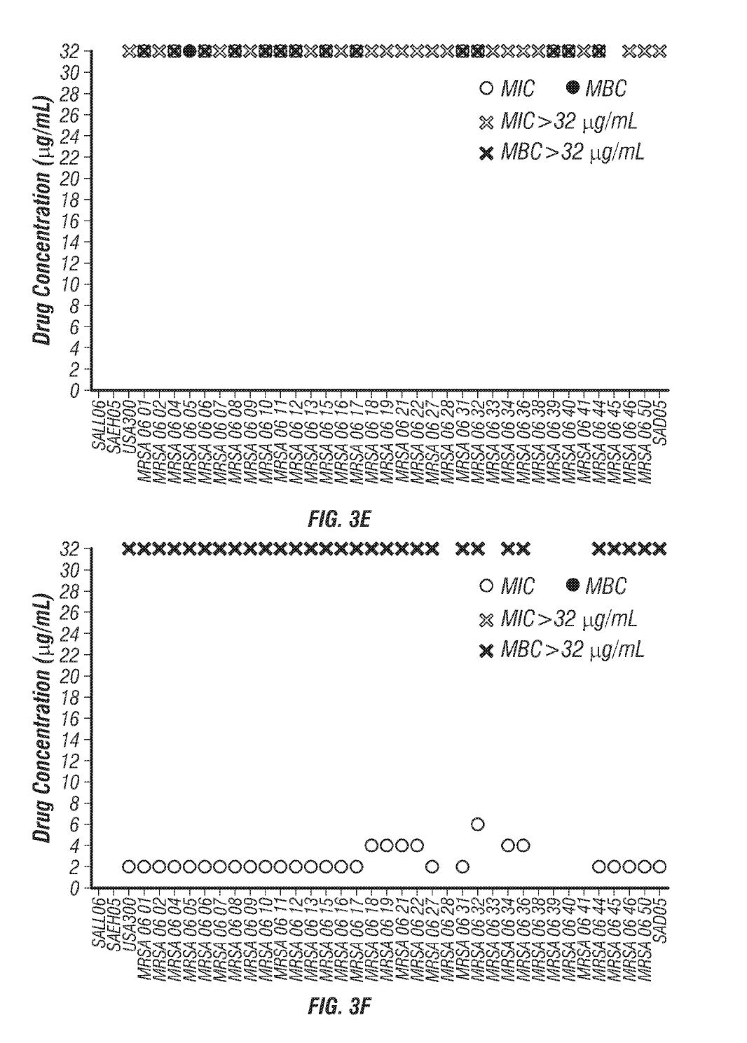

Anti-microbial activity C58 and C59 was tested against 38 clinical isolates of MRSA using a standard broth microdilution method described previously in a standard protocol using Meuller-Hinton (MH) broth (Wright et al., 2012 and Leid et al., 2012) to determine minimum bacteriostatic and bactericidal concentrations (MIC and MBC). Antimicrobials used for treatment of MRSA, including the current gold standard vancomycin hydrochloride, comprised the controls. These results are shown in FIG. 3 and are summarized as MIC₉₀ and MBC₉₀ in Table 2 (below). These values represent the concentration of antimicrobial agent required to inhibit the growth (MIC₉₀) or kill (MBC₉₀) 90% of the tested bacterial strains.

TABLE 2
MIC₉₀ and MBC₉₀ values for antimicrobial agents tested against
38 MRSA strains using a standard broth microdilution method.

| | MIC₉₀ (μg/mL) | MBC₉₀ (μg/ml) |
|---|---|---|
| C58 | 2 | 4 |
| C59 | 2 | 6 |
| Vancomycin hydrochloride | 2 | 16 |
| Clindamycin hydrochloride | >32 | >32 |
| Daptomycin hydrochloride | >32 | >32 |
| Linezolid | 4 | >32 |

Biaryl compounds C58 and C59 exhibit superior bacteriostatic activity compared with clindamycin hydrochloride, daptomycin hydrochloride, and linezolid, and comparable activity to the current gold standard, vancomycin hydrochloride and daptomycin hydrochloride (Table 2). However, the bactericidal activity of C58 and C59 is superior to all the commercially available antimicrobials, including vancomycin (Table 2). In addition, at least 10 of the 38 strains have been identified as potentially hypermutable strains and exhibit highly variable and inconsistent bactericidal concentrations upon incubation with vancomycin. These strains are MRSA 0601, 0611, 0612, 0627, 0628, 0631, 0633, 0638, 0644, and 0645. This phenomenon is limited to vancomycin and biaryl compounds eradicate these strains at MBC₉₀ concentrations (FIG. 3). Clinical and Laboratory Standards Institute (CLSI) recommends use of cation-adjusted MH broth containing Ca⁺² ions to determine the antimicrobial activity of daptomycin hydrochloride. The results demonstrated in Table 2 and FIG. 3 were generated using MH broth, which is used for all the antimicrobial agents. The activity of daptomycin against the 38 MRSA strains using cation adjusted MH broth was determined. These results demonstrate an MIC₉₀ and MBC₉₀ of 2 μg/mL, which is comparable to the antimicrobial activity of biaryls.

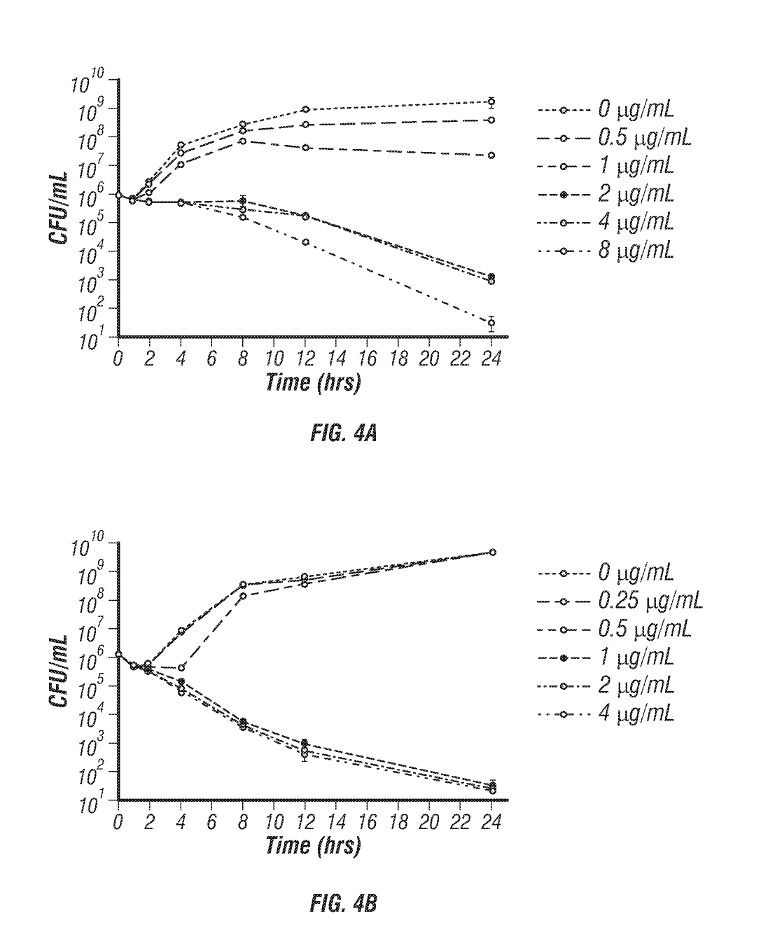

The rate of bacterial eradication for C59 and vancomycin was then measured using planktonic, log-phase MRSA strain USA300 (FIGS. 4A-4B) over a period of 24 hours. These experiments show an absence of dose response for vancomycin, while C59 effectively reduces the bacterial burden in a dose-dependent manner. A reduction in bacterial burden is observed at sub-MIC concentration as low as 0.5 μg/mL compared with untreated bacteria and complete killing is observed at 8 μg/mL. These results also suggest a higher risk of resistance development with vancomycin compared to biaryl compounds.

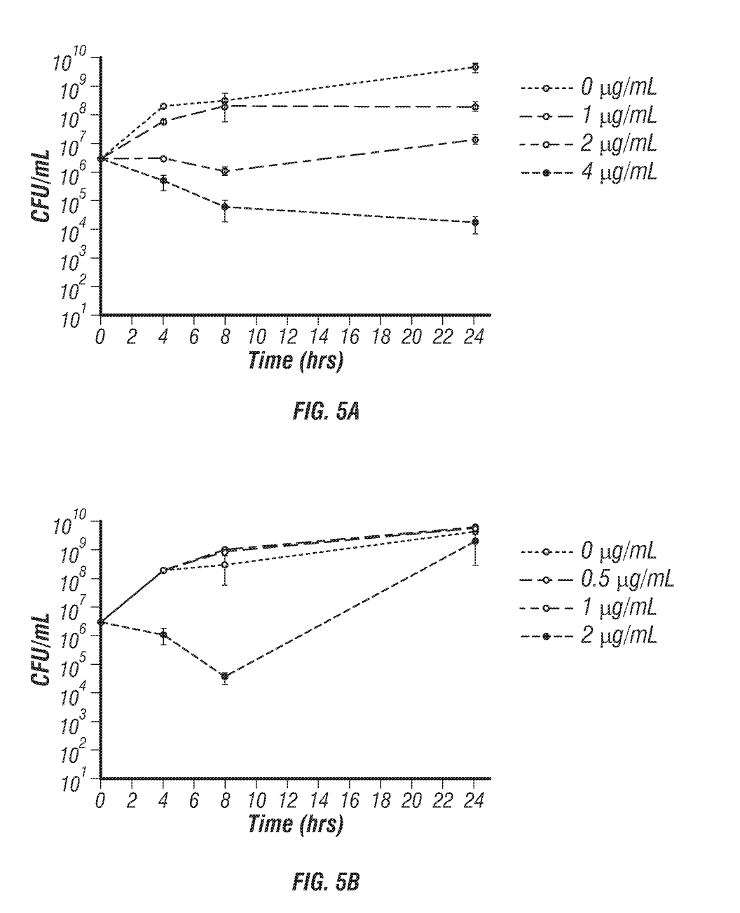

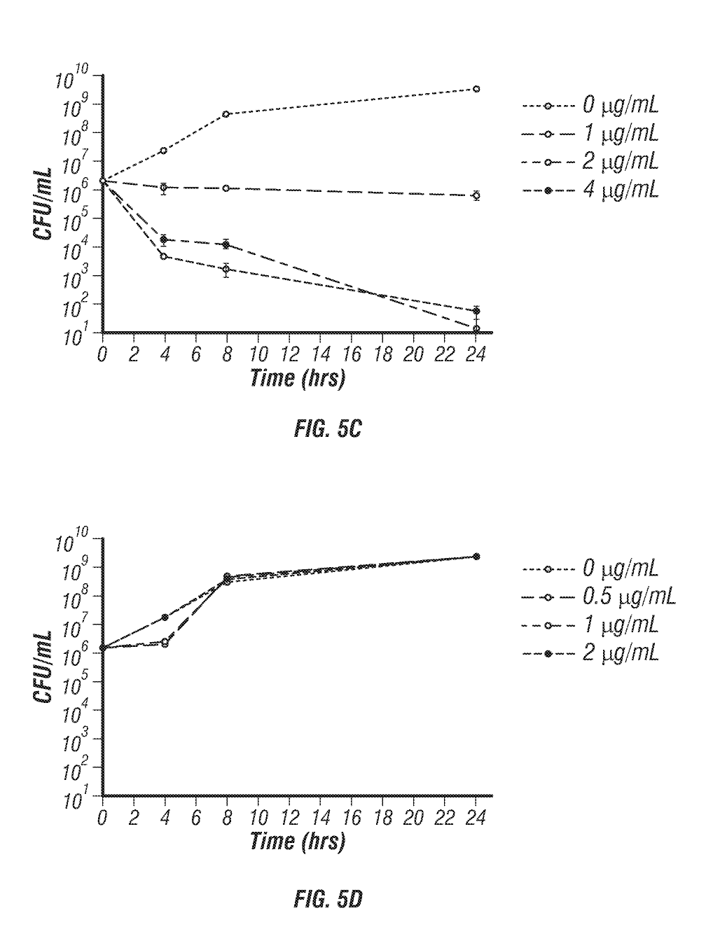

Next, a similar kill kinetics experiment was performed, but using stationary-phase bacteria instead of log-phase actively growing bacteria using MRSA strains USA300 and SALL06. The activity of vancomycin observed with log-phase bacteria (FIGS. 4A-4B) is not preserved with stationary-phase bacteria as vancomycin fails to eradicate the bacterial burden completely (FIGS. 5A-5D). At 24 hours, no difference is observed in bacterial counts between groups treated with 0.5-2.0 Kg/mL vancomycin and untreated controls (FIGS. 5B and 5D). The activity of C59 appears to be slightly diminished in comparison with its activity against log-phase bacteria; however, C59 continues to eradicate bacteria and a dose response is maintained at concentrations ranging from sub-MIC to MBC. Thus, biaryl compound C59 exhibits superior antimicrobial efficacy against stationary-phase bacteria compared to the current gold standard, vancomycin hydrochloride.

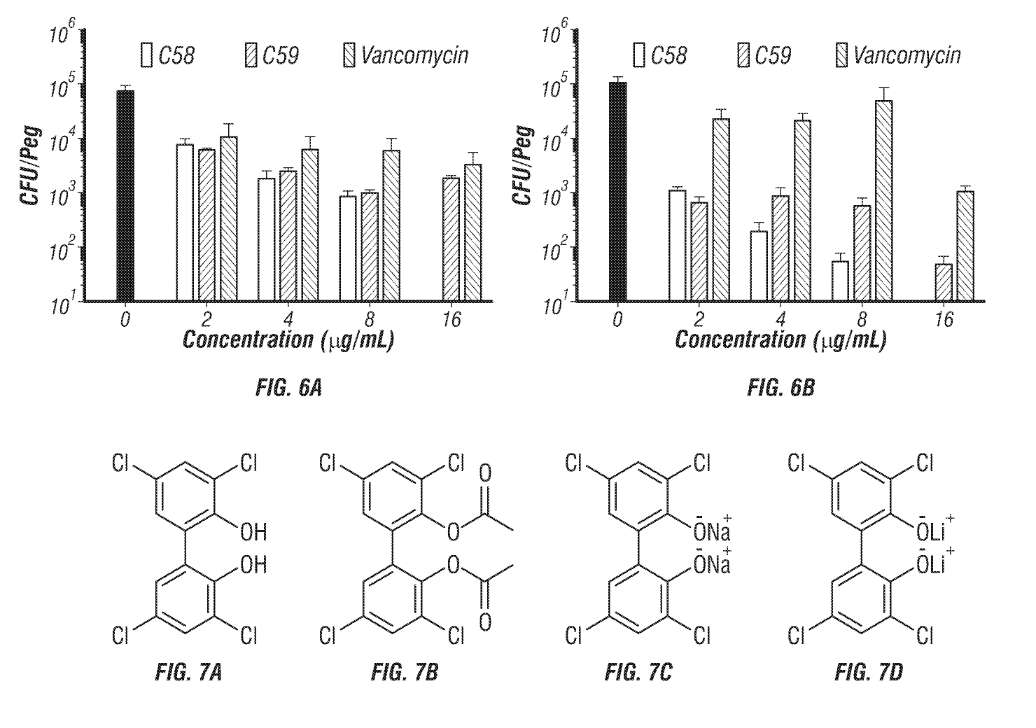

Bacterial infections often lead to formation of biofilms at the site of infection (Olson et al., 2002). These biofilms are safe havens for bacteria as they allow bacteria to survive in a stationary-phase. Thus, an effective antimicrobial agent must be able to penetrate and eradicate stationary-phase bacteria hidden inside a biofilm. An MBEC™ High-throughput assay that allows bacteria to form biofilms was used to mimic biofilms formed at the site of infection. First, 8 MRSA strains were screened for their ability to consistently form biofilms and MRSA strains SAD05 and 0632 were identified as promising candidates for further experiments. Bacterial biofilms formed using the MBEC™ High-throughput assay plate using MRSA strains SAD05 and 0632 were treated with biaryl compounds or vancomycin for 6 hours, and colony-forming units of bacteria for each treatment (peg) were measured to determine activity. Biaryl compounds show a significant reduction in bacterial burden at concentrations as low as 2 μg/mL compared to vancomycin (FIGS. 6A-6B) and biaryl compound C58 appears to be more potent compared to C59. C58, but not vancomycin completely eradicates bacteria in a biofilm at a concentration of 16 μg/mL after a six-hour incubation with MRSA strains, SAD05 and 0632. Lack of activity of vancomycin against biofilm mode bacteria is consistent with published reports (Raad et al., 2007 and Rose and Poppen 2009). Thus, biaryl compounds show superior efficacy compared to vancomycin against planktonic bacteria in a wide range of conditions as well as against stationary-phase bacteria in biofilm mode.

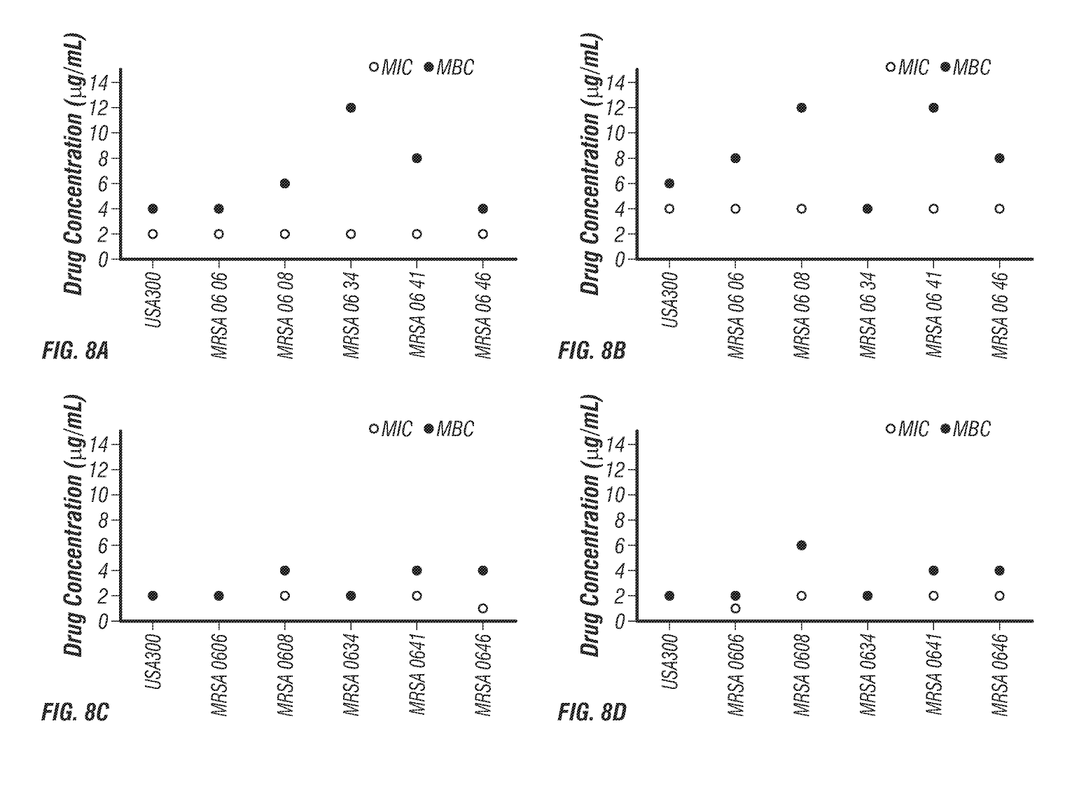

Six MRSA strains, USA300, 0606, 0608, 0634, 0641, and 0646 were chosen based on their activity against biaryl compounds and vancomycin to determine structure-activity relationship of biaryl compounds. The effect of phenolic group on the activity of biaryl compounds was tested by modifying C59. First, the phenolic group was protected by acetylation to yield an acetylated C59 (C59-2) (FIG. 7B). Additional variants of C59, in the form of sodium and lithium salts were synthesized by incubation with sodium hydroxide and lithium hydroxide (FIGS. 7C and 7D). Antimicrobial activity of these deprotonated, water-soluble sodium (C59Na) and lithium (C59Li) salts, and acetylated derivative of C59 was tested against 6 MRSA strains and compared to the parent compound, C59 (FIGS. 8A-8D). Results from this comparative experiment indicate the contribution of phenolic group/phenoxide ion to the antimicrobial activity. Reduction of activity is observed upon protecting the phenolic group with an acetyl group (C59-2) compared to the parent compound C59 as evident by higher MIC and MBC values (FIGS. 8A-8D). On the other hand, deprotonated sodium and lithium salts, show enhanced activity compared to C59 under similar conditions.

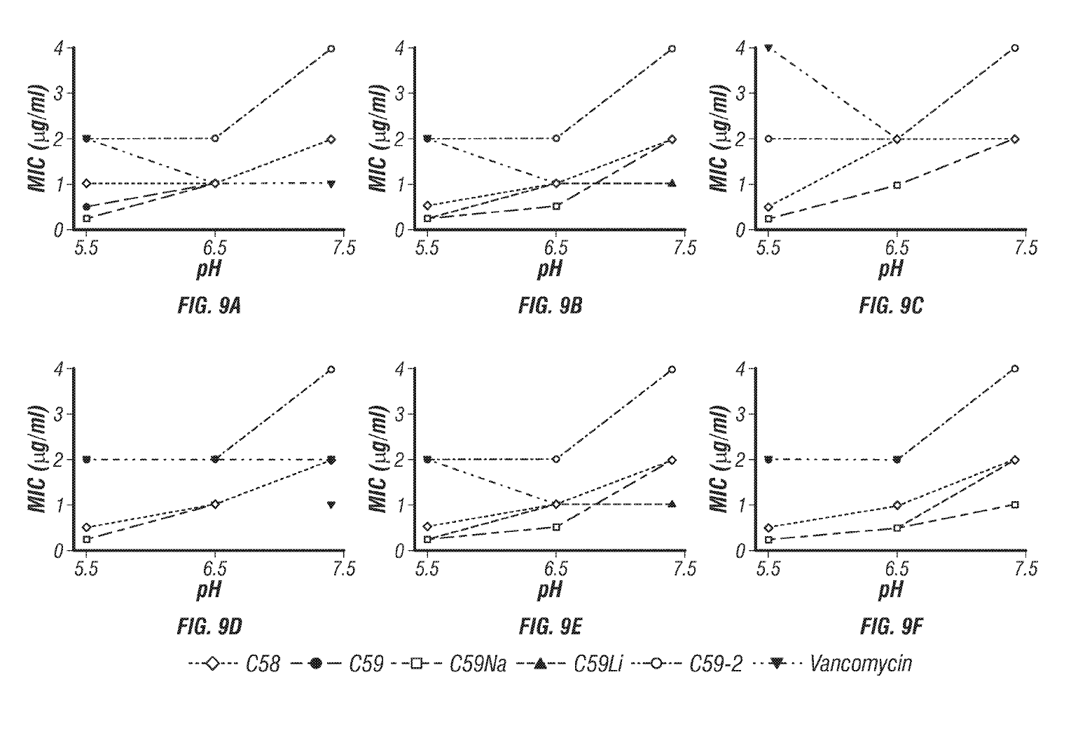

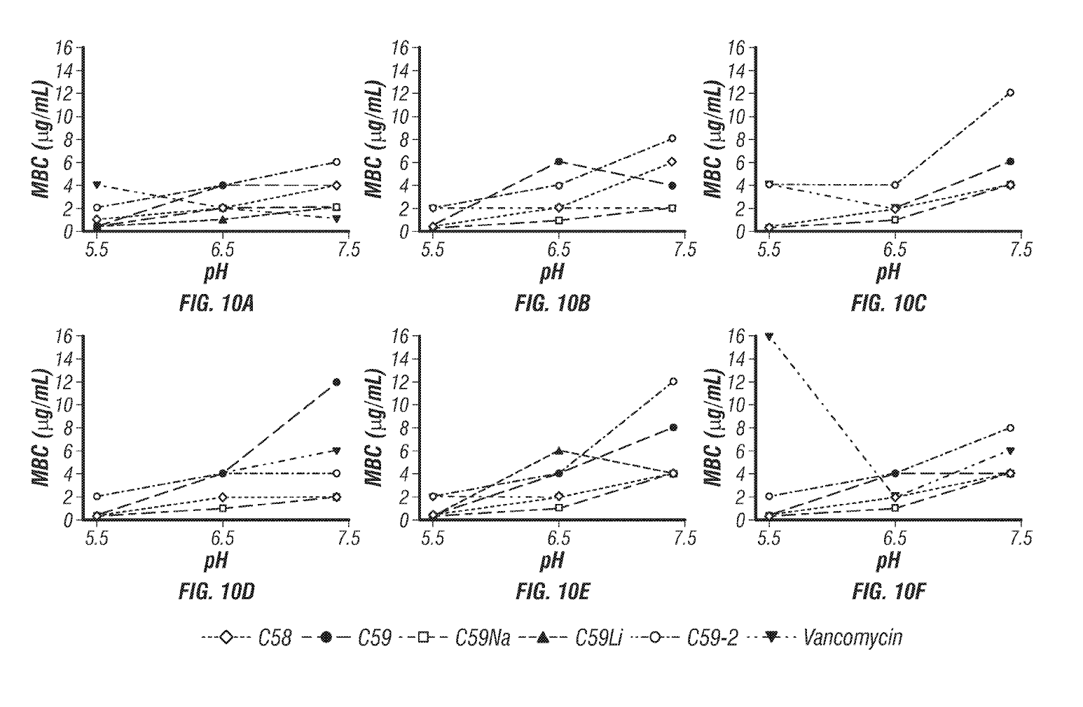

Further investigation into activity of these compounds under acidic pH conditions also points towards the role of phenolic group. An effective antimicrobial should be active under a wide range of pH conditions, including the physiological pH of 7.4. Localized pH in an infection is often lower than the physiological pH, and may be as low as 5.5 (Radovic-Moreno et al., 2012). Additionally, pH varies among organs (Radovic-Moreno et al., 2012). For example, pH in the bladder or on the skin is found to be between 4.5 and 8.0, or 4.0 and 5.5, respectively (Radovic-Moreno et al., 2012). The activity of biaryl compounds, C58, C59, C59-2, C59Na, and C59Li, as well as vancomycin control were tested under acidic pH conditions (FIGS. 9A-9F and 10A-10F). The activity of all biaryl compounds is enhanced by at least four- to eight-fold with reduction in pH from 7.4 to 5.5. Vancomycin on the other hand does not exhibit any improvement and in several cases shows deteriorated activity as the pH changes from 7.4 to 5.5.

It is believed the activity of the phenolic group can be partly attributed to the presence of electron withdrawing halogens in an ortho- and para-position to the phenol in C58 and C59. These halogens increase the acidity of the phenolic group, which is lost when the phenol is protected using an acetyl group. In addition, the sodium and lithium salts add to the polarity of the phenolic group and complement the activity, which indicates that the ease of ionization of the phenolic group to form a phenoxide ion is critical for antimicrobial activity. Thus, the electron withdrawing halogen groups, phenolic groups, and their position relative to each other on the phenyl ring seem to play a crucial role in the mechanism of action for biaryl compounds.

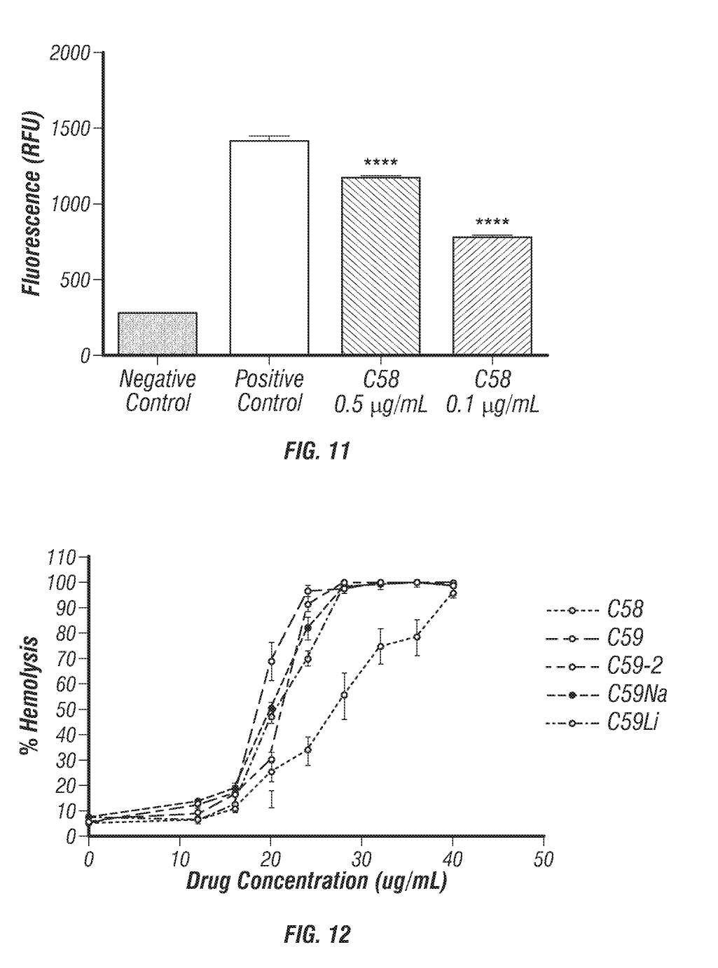

Biaryl compounds and their analogues have been reported to be membrane active agents (Isnansetyo and Kamei, 2003 and Kamei et al., 2003). An experiment was performed to verify the membrane permeabilization activity of biaryl compound. C58 using a fluorescent dye, 3,3′-Dipropylthiacarbocyanine iodide (DiSC₃(5)). The fluorescence signal from DiSC₃(5) is in a quenched state when it interacts with the hydrophobic region of the cell membrane. Upon disruption of the membrane, DiSC₃(5) is released and regains its fluorescent activity, which is measured as a fluorescence signal (RFU). Results from this experiment show positive membrane disruption of MRSA strain USA300 after incubation with 0.1 and 0.5 μg/mL C58 as well as positive control, 100 nM 3,3′,4′,5-tetrachlorosalicylanilide (FIG. 11). These results indicate the mechanism of action to be cell membrane disruption.

Toxicity of biaryl compounds was tested against erythrocytes as well as eukaryotic epithelial, dermal, and macrophage cell lines in vitro. Hemolytic activity of biaryl compounds, C58, C59, C59-2, C59Na, and C59Li was determined by incubating red blood cells (erythrocytes from sheep blood) with biaryl compounds for 18 hours. None of the biaryl compounds are hemolytic at therapeutic concentrations with the onset of hemolysis occurring between 16 and 20 μg/mL (FIG. 12) for each of the biaryl compounds. In contrast, FDA approved membrane active agents Gramicidin D and Amphotericin B result in 60% and 70% hemolysis at 50 and 10 μg/mL concentrations after just two hour incubation with erythrocytes (Isnansetyo and Kamei, 2003).

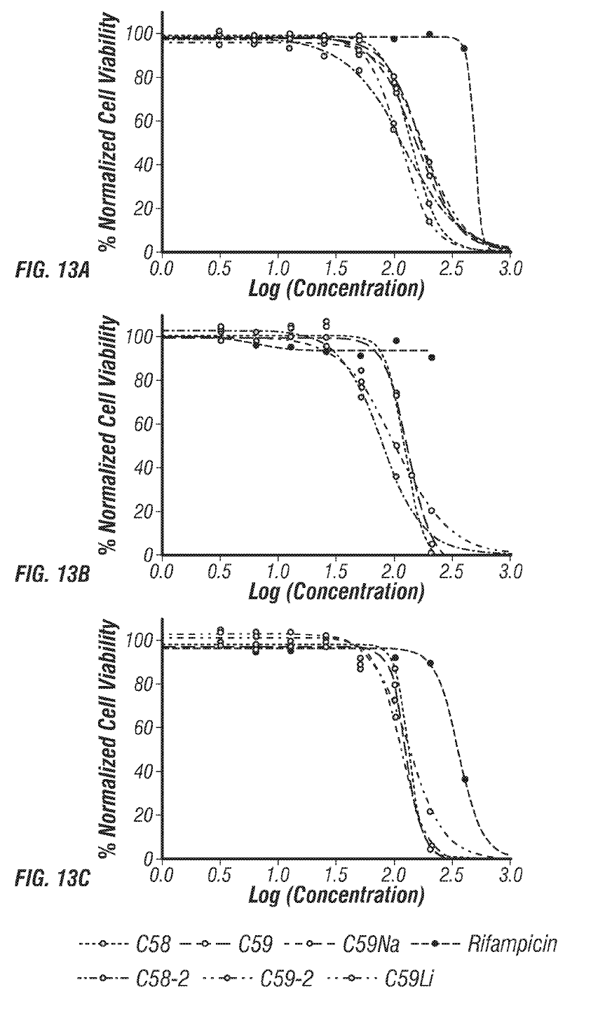

Finally, toxicity of biaryl compounds was tested against human lung airway epithelial cells (16HBE), human dermal fibroblasts, and J774.A₁ murine macrophages using an ALAMARBLUE® Cell Viability Assay. Biaryl compounds, C58, acetylated C58 (C58-2), C59, C59-2, C59Na, and C59Li were incubated with cells for 24 hours and an ALAMARBLUE® Cell Viability Assay was performed to determine cell viability relative to untreated controls (FIGS. 13A-13C). Cell viability curves were analyzed using non-linear curve fit to yield the inhibitory concentration at median cell survival (LD₅₀) for all cell lines (Table 3). These data demonstrate a lack of toxicity towards the eukaryotic cell lines tested at concentrations at which the antimicrobial activity is exerted. The MBC₉₀ of C58 and C59 at 4 and 6 μg/mL is at least five fold lower than the LD₅₀ of 60 and 40 μg/mL, respectively. Additionally, the sodium and lithium salts of C59 demonstrate enhanced antimicrobial activity against MRSA while conserving the safety profile of C59. Thus, these compounds provide a large therapeutic window for treatment.

In addition to MRSA, biaryl compounds demonstrate antimicrobial activity against other Gram-positive bacteria. Initial screening performed using Enterococcus faecalis (E. faecalis) and Rhodococcus equi (R. equi) demonstrates improved activity compared to clinically used antibiotics. Activity of all tested biaryl compounds is similar for both vancomycin sensitive and resistant E. faecalis strains (Table 4). Additionally, vancomycin fails to eradicate both sensitive and resistant E. faecalis strains, while biaryl compounds successfully eradicate these bacteria at clinically achievable concentrations.

TABLE 4
MIC and MBC values for antimicrobial agents tested against E. faecalis
strains 49532 and 51575 using a standard broth microdilution method.

| | 49532 | | 51575 | |
|---|---|---|---|---|
| | MIC | | MIC | MBC |
| | (μg/mL) | MBC (μg/mL) | (μg/mL) | (μg/mL) |
| C58 | 6 | 12 | 6 | 12 |
| C59 | 8 | 16 | 8 | 16 |
| C59Na | 12 | 24 | 12 | 24 |
| Vancomycin | 1 | >512 | 192 | >192 |

The activity of biaryl compounds against R. equi is of particular interest. While R. equi primarily infects foals between ages 3 and 5 months, it shares its genera with Mycobacterium, and the mean identity of the shared core orthologs between R. equi and Mycobacterium tuberculosis (Mtb) is 64.6%. Furthermore, R. equi, analogous to Mtb, is an intracellular bacterium and often resides in macrophages. Thus, R. equi can also serve as a model to identify lead candidates for MtB treatment. The activity of biaryl compounds against four R. equi strains, 701+, 701−, 703, and 5331 is listed in Table 5. Biaryl compounds exhibit comparable bacteriostatic activity and superior bactericidal activity to clinically used rifampicin. Their activity against R. equi is at much lower concentrations compared to the IC₅₀ towards macrophages, providing a large therapeutic window for treatment. These preliminary screening experiments show tremendous potential and screening will be expanded to include the rest of the library to identify lead candidates and develop structure-activity relationship for effective eradication of R. Equi, E. faecalis, and Mtb.

TABLE 5
MIC and MBC values for antimicrobial agents tested
against R. equi strains 701+, 701−, 703, and 5331
using standard broth microdilution method.

| Concen- | | | | | | | | |
|---|---|---|---|---|---|---|---|---|
| tration | 701+ | | 701− | | 703 | | 5331 | |
| (μg/mL) | MIC | MBC | MIC | MBC | MIC | MBC | MIC | MBC |
| C58 | 1 | 4 | 1 | 8 | 0.5 | 8 | 1 | 4 |
| C59 | 2 | 4 | 2 | 16 | 2 | 8 | 2 | 4 |
| C59-2 | 4 | 16 | 4 | 16 | 2 | 16 | 4 | 16 |
| C59Na | 2 | 16 | 8 | 32 | 1 | 8 | 2 | 8 |
| Rifampicin | 0.5 | >192 | 0.5 | >192 | 0.125 | 80 | 0.125 | 32 |

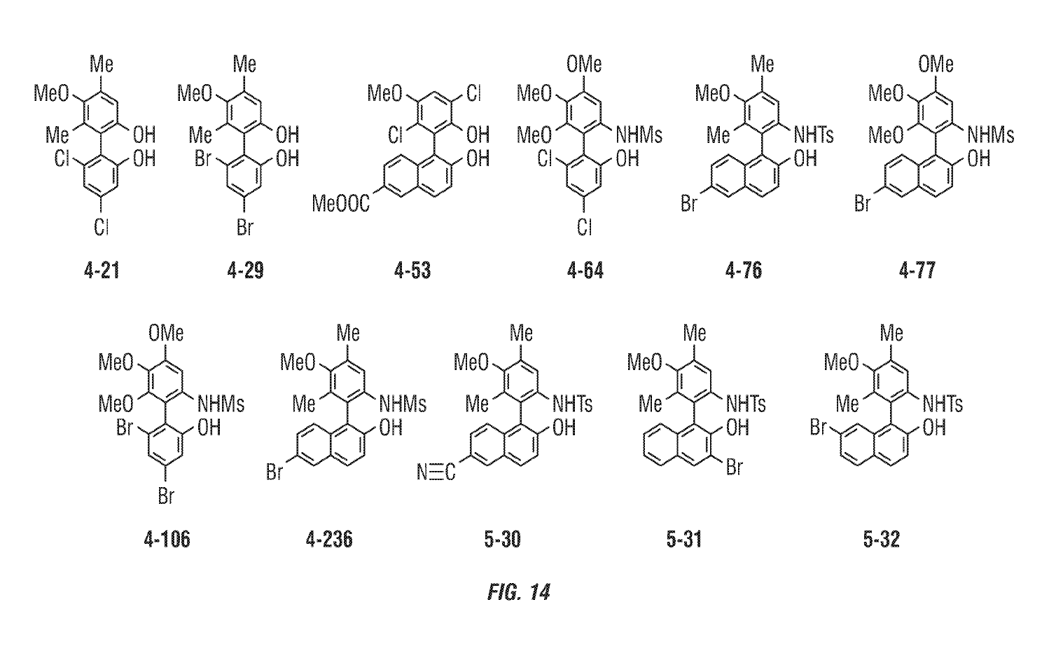

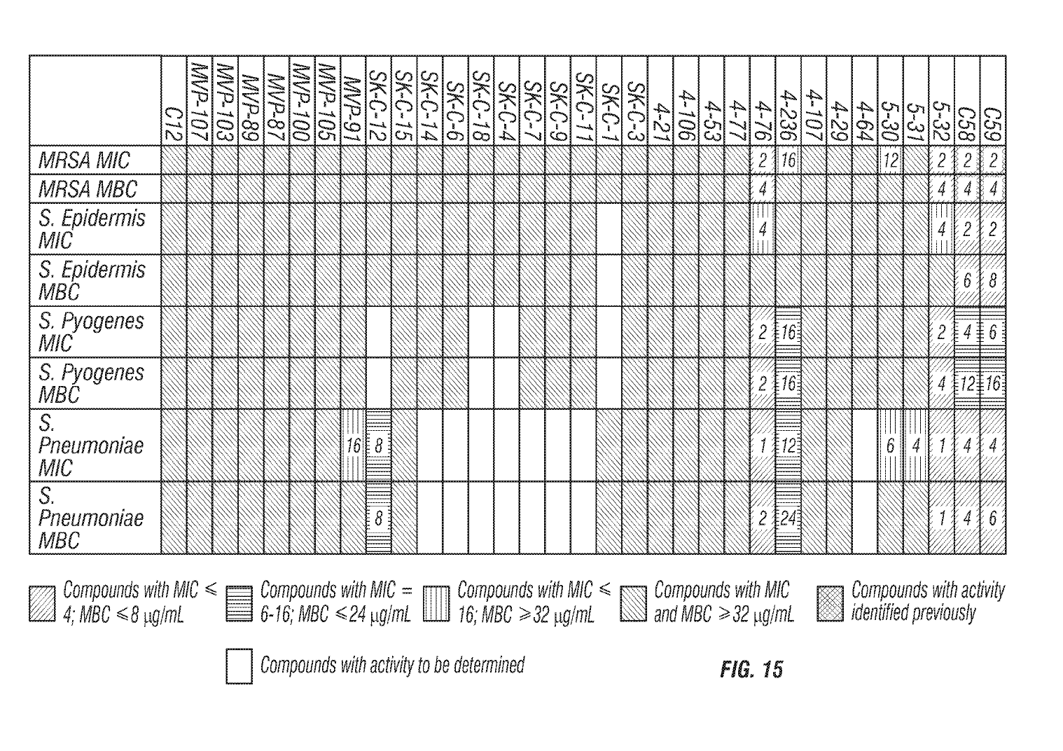

An expanded library of biaryl compounds (FIG. 14) were synthesized and evaluated against a representative methicillin-resistant Staphylococcus aureus (MRSA) strain, USA 300, using the standard CLSI broth microdilution method to identify active compounds as described previously. All experiments were performed in presence of 2.5% DMSO according to the CLSI guidelines for hydrophobic agents, unless otherwise stated. In addition to MRSA, compounds from this library have also been screened against other Gram-positive pathogens, Streptococcus pyogenes, Streptococcus pneumoniae, and Staphylococcus epidermidis to identify pathogen-specific lead candidates. Using these screens, two new lead candidates, 4-76 and its structural isomer, 5-32, were identified with potent anti-MRSA activity (MIC≤4; MBC≤8 μg/mL), as well as activity against other Gram-positive pathogens (FIG. 15). Moreover, C58 and C59 also demonstrate potent antimicrobial activity against S. epidermidis and S. pneumoniae. Additional compounds identified using these screens include, pathogen-specific bacteriostatic agents (MIC≤16; MBC≥32 μg/mL) and compounds with moderate activity (MIC=6-16; MBC≤24 μg/mL). These compounds are 4-76 and 5-32, MVP-91, 5-30, and 5-31, as well as 4-236 and 5-30, with bacteriostatic activity against S. epidermidis, S. pneumoniae, and MRSA, respectively. Compounds, C58 and C59, with activity against MRSA, also demonstrate moderate antimicrobial activity against S. pyogenes. Finally, SK-C-12 and 4-236 demonstrate moderate activity against S. pneumoniae.

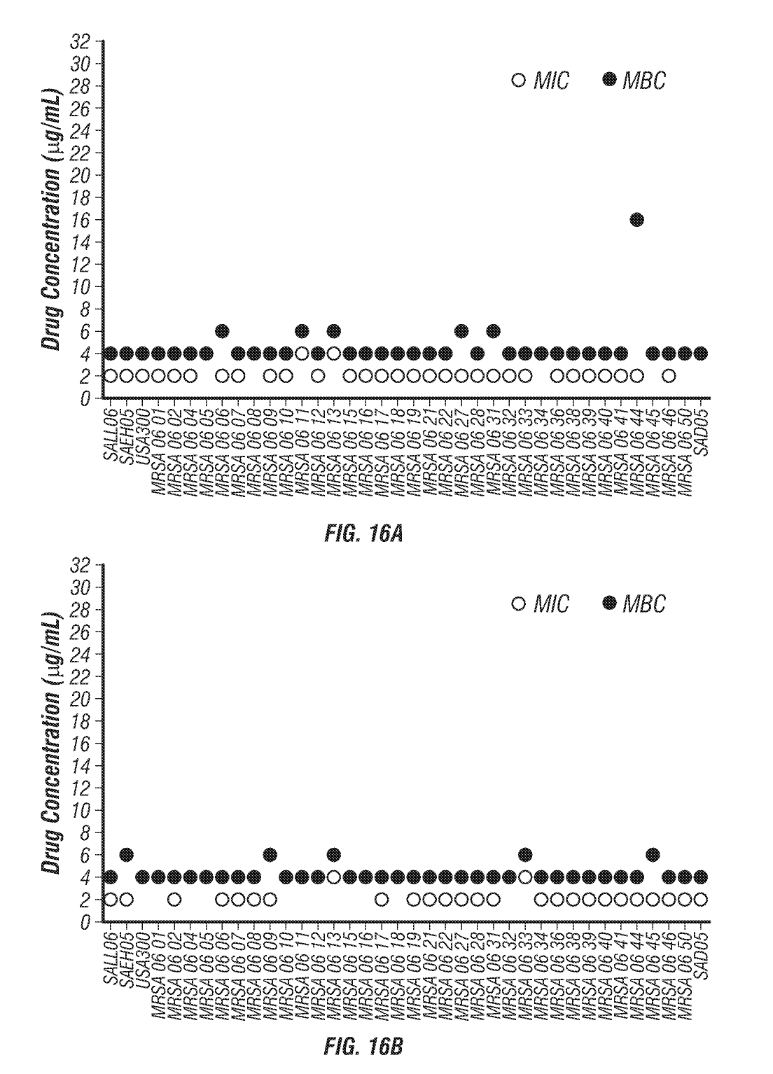

Next, the minimum inhibitory and bactericidal concentrations (MIC and MBC) of the active lead candidates were evaluated against panels of each Gram-positive pathogen and compared with standard-of-care (SoC) antimicrobials, including vancomycin. Reported above are MICs and MBCs for C58, C59, and SoC comparators, vancomycin hydrochloride, clindamycin hydrochloride, daptomycin hydrochloride, and linezolid against MRSA. The activity of the two new candidates, 4-76 and 5-32, against MRSA is represented in FIG. 16. The MIC₉₀ and MBC₉₀ values representing concentrations required to inhibit or eradicate 90% of the strains (MIC₉₀ and MBC₉₀) calculated from these data are listed in Table 6. These two lead candidates demonstrate comparable bacteriostatic and superior bactericidal activity over vancomycin and linezolid. Both compounds have superior antimicrobial activity compared with clindamycin (MIC₉₀ and MBC₉₀≥32 μg/mL).

TABLE 6
MIC₉₀ and MBC₉₀ values for antimicrobial agents tested against 38
MRSA strains usinga standard broth microdilution method.

| | MIC₉₀ (μg/mL) | MBC₉₀ (μg/mL) |
|---|---|---|
| 4-76 | 4 | 6 |
| 5-32 | 4 | 6 |

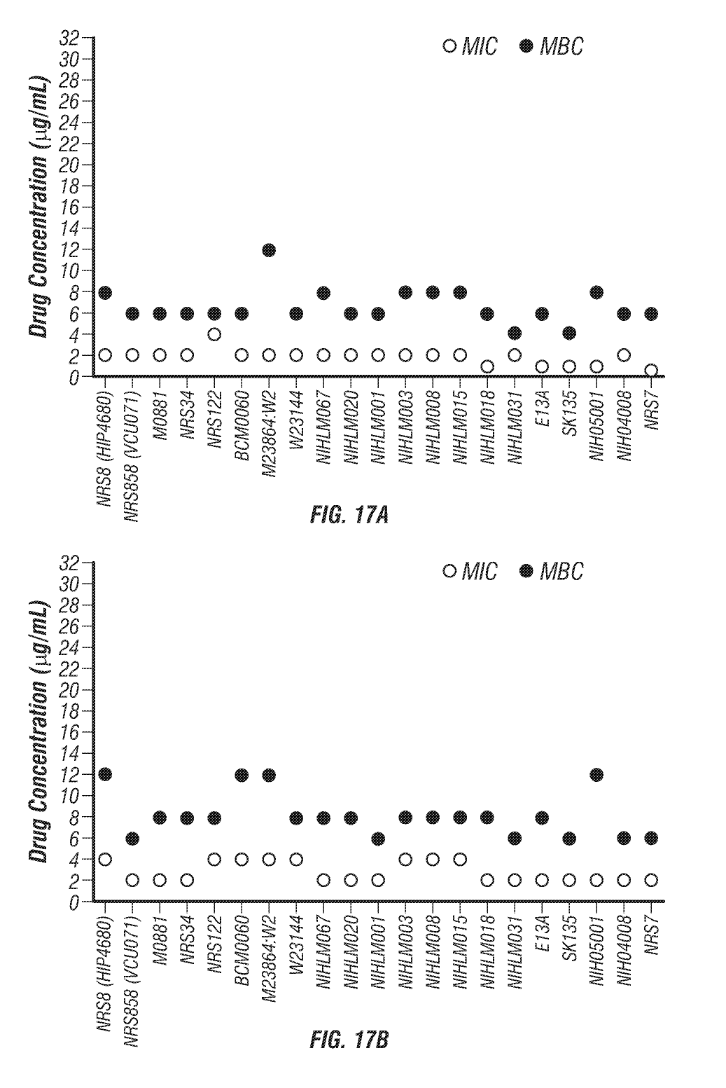

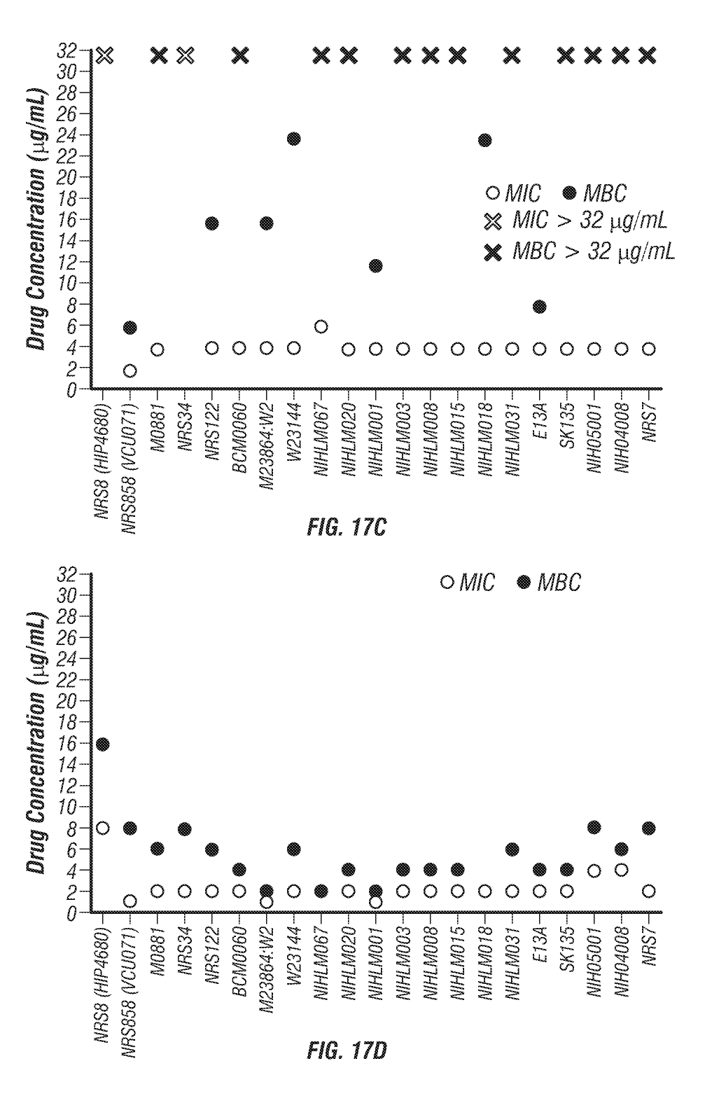

In addition to MRSA, C58, C59 also demonstrate potent bactericidal activity against S. epidermidis, whereas, 4-76 and 5-32, demonstrate bacteriostatic activity against S. epidermidis (FIG. 15). The activity of C58, C59, and 4-76 was determined against a panel of 21 S. epidermidis strains and compared with SoC, vancomycin hydrochloride (FIGS. 17A-17D). We are currently in the process of evaluating the MICs and MBCs for 5-32. All three lead candidates demonstrate MIC₉₀ values comparable to vancomycin, however, only C58 and C59 demonstrate MBC₉₀ values comparable to vancomycin, while 4-76 primarily demonstrates bacteriostatic activity (Table 7).

TABLE 7
MIC₉₀ and MBC₉₀ values for antimicrobial agents tested against
21 S. epidermidis strains using standard broth microdilution method.

| | MIC₉₀ (μg/mL) | MBC₉₀ (μg/mL) |
|---|---|---|
| C58 | 2 | 8 |
| C59 | 4 | 12 |
| 4-76 | 4 | >32 |
| Vancomycin hydrochloride | 4 | 8 |

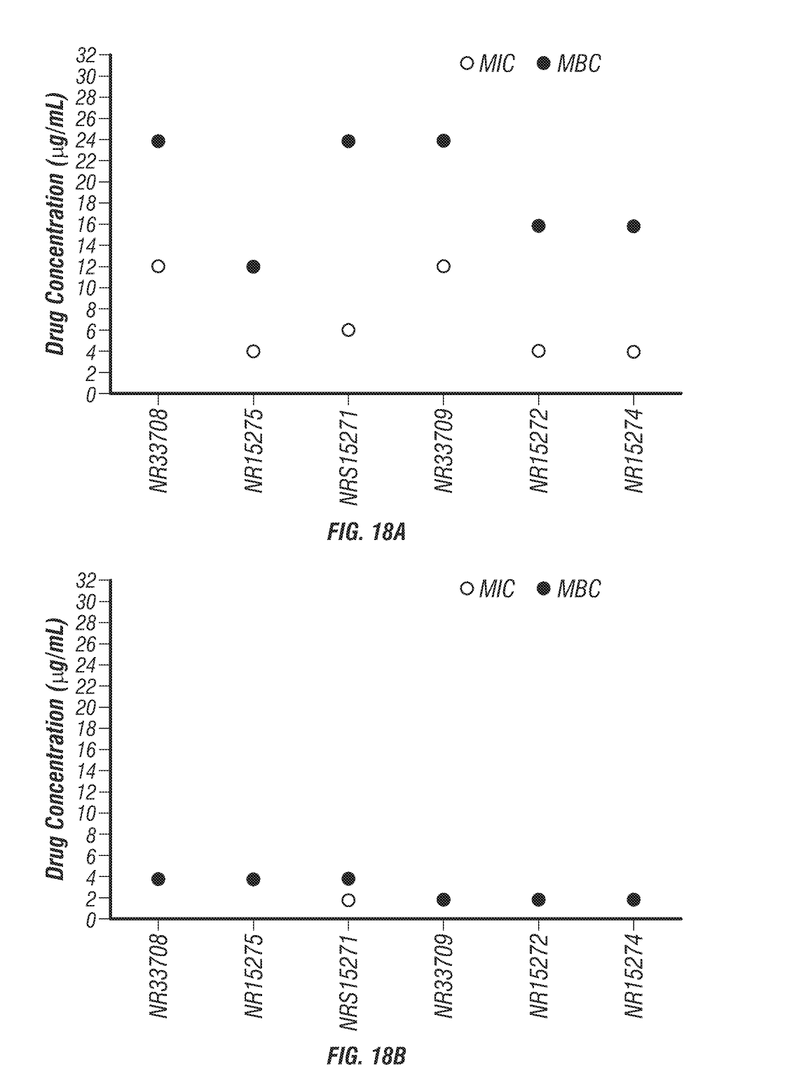

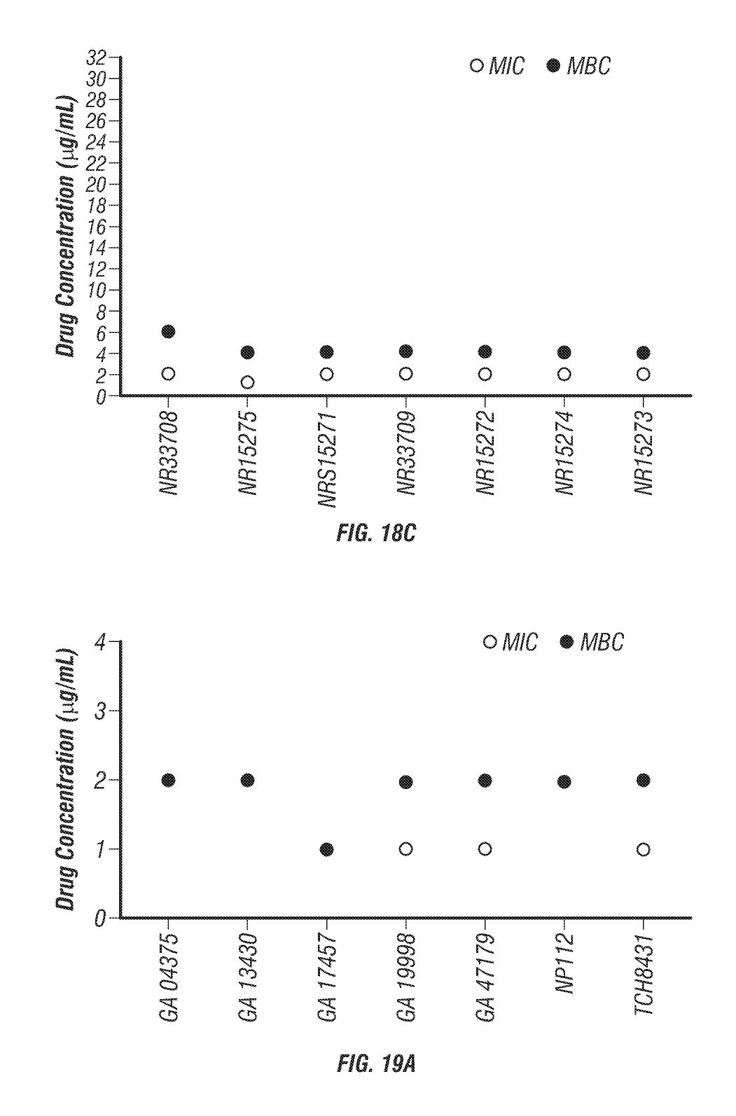

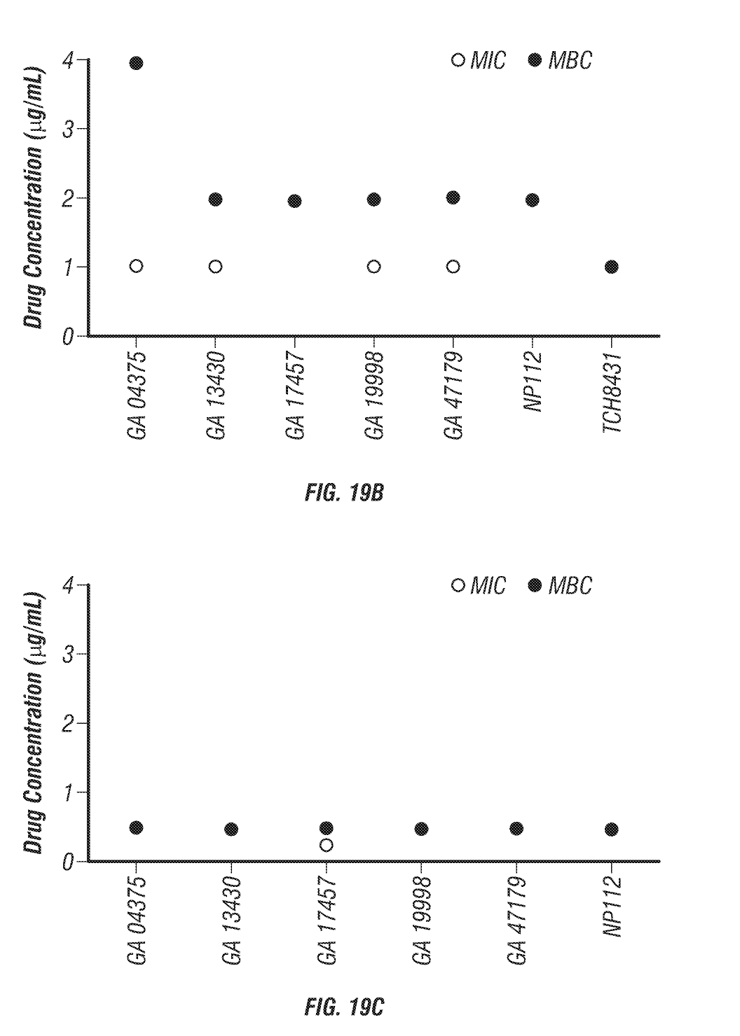

The activity of compounds against Gram-positive pathogens, S. pyogenes and S. pneumoniae, identified through the preliminary screen (FIG. 15) was evaluated against a panel of these pathogens. Preliminary results demonstrate MICs and MBCs for 4-76 and 5-32 ranging between 2 and 4 μg/mL and 2 and 6 μg/mL, respectively against seven S. pyogenes strains (FIGS. 18A-18C). These compounds are more potent compared with C59, which demonstrates MICs and MBCs ranging between 2-12 μg/mL and 12-24 μg/mL, respectively. Similarly, 4-76 and 5-32 demonstrate MICs and MBCs ranging between 1 and 2 μg/mL and 1 and 4 μg/mL, respectively against seven S. pneumoniae strains (FIGS. 19A-19C). These results are comparable to vancomycin, which demonstrates MICs and MBCs ranging between 0.25 and 0.5 μg/mL.

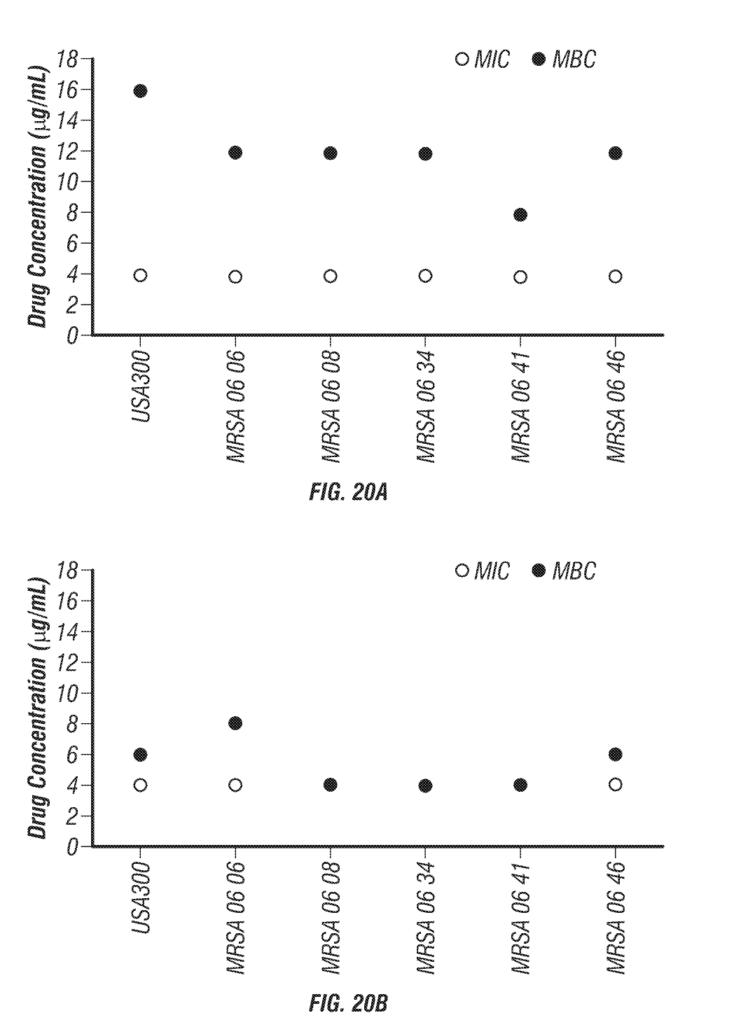

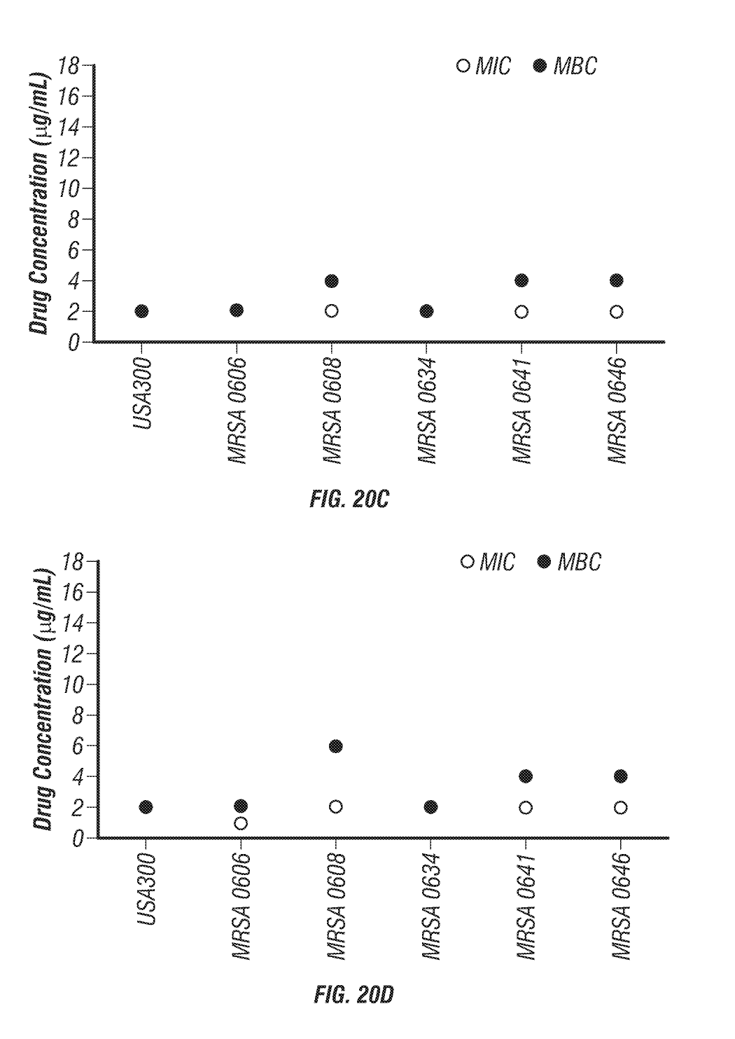

Water soluble sodium and lithium salts of C59 (C59Na and C59Li) and their antimicrobial activity against six MRSA strains are reported above. A sodium salt of C58 (C58Na) was prepared and evaluated efficacy of this compound against these six MRSA strains. These results demonstrate C58 to have superior activity over the sodium salt C58Na (FIGS. 20A & 20B). In contrast, C59Na and C59Li demonstrate superior activity over C59 (FIG. 8). The poor activity of C58Na may be a result of poor stability of C58Na compared with C59Na and C59Li. In addition, all MIC and MBC determinations are performed in presence of 2.5% DMSO to solubilize hydrophobic antimicrobials according to CLSI guidelines. The ability of C58Na, C59Na, and C59Li to inhibit and eradicate MRSA in absence of DMSO were also tested to demonstrate their high solubility in aqueous media. Both sodium salts, C58Na and C59Na, as well as the lithium salt of C59, C59Li, demonstrate at least comparable activity in the absence of DMSO, providing a potential water-soluble formulation for delivery of these compounds.

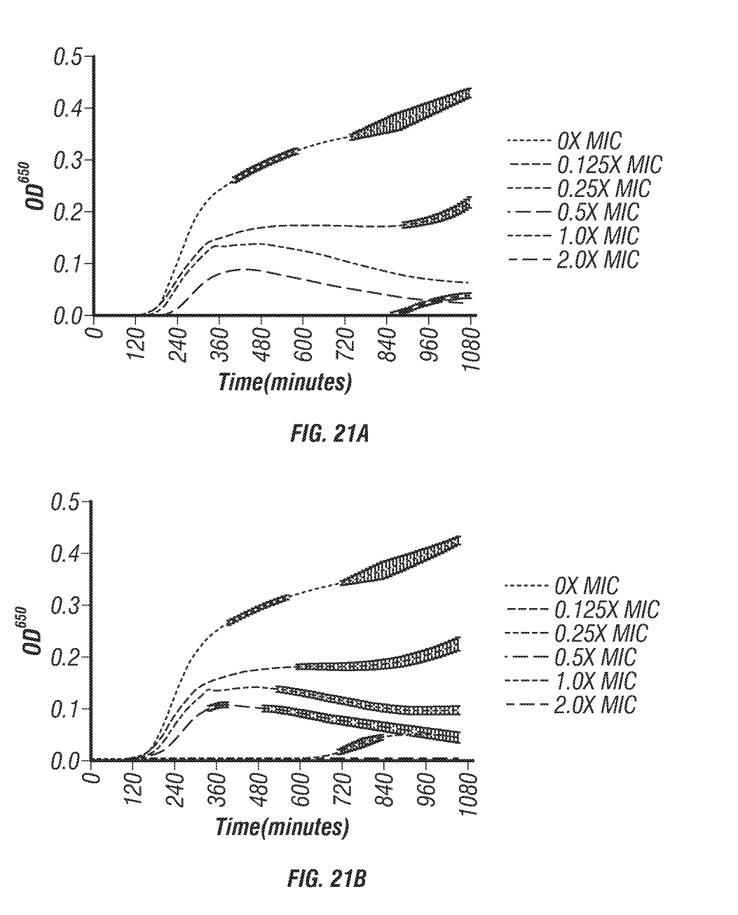

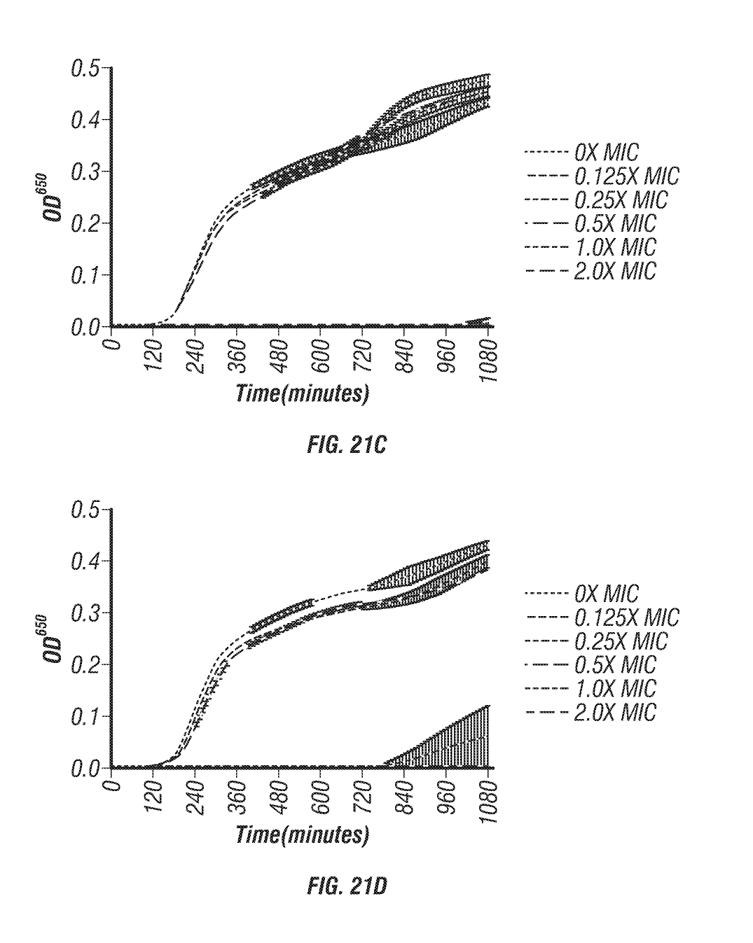

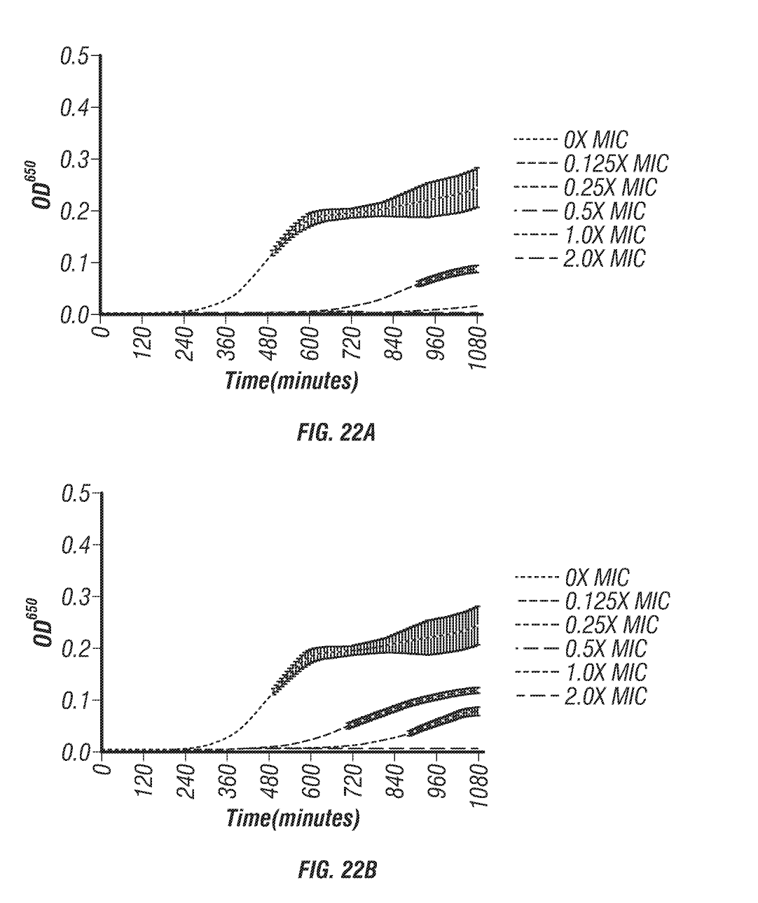

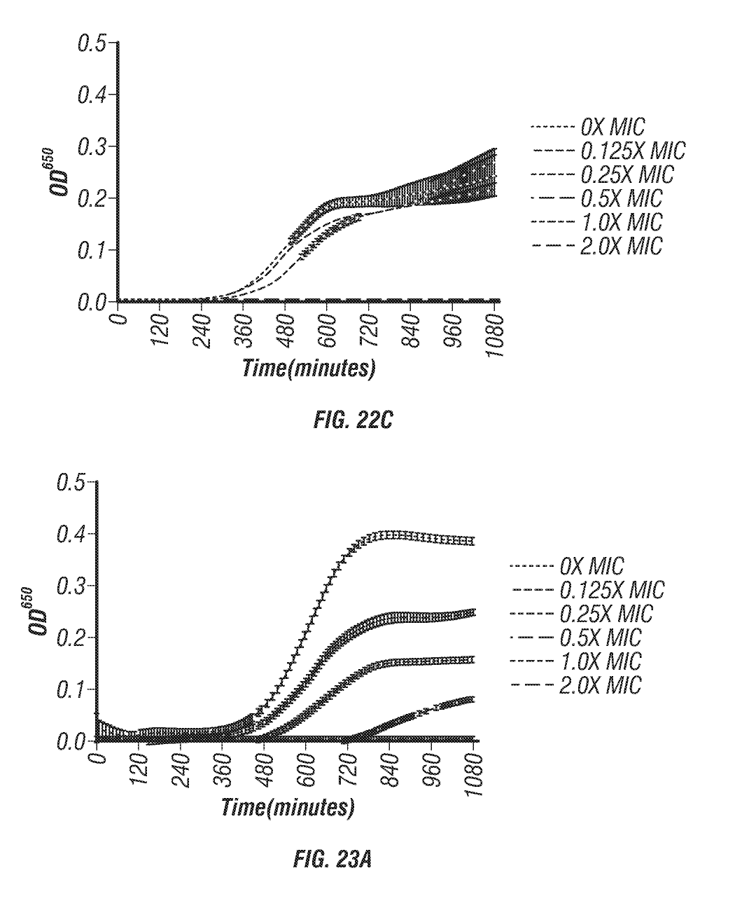

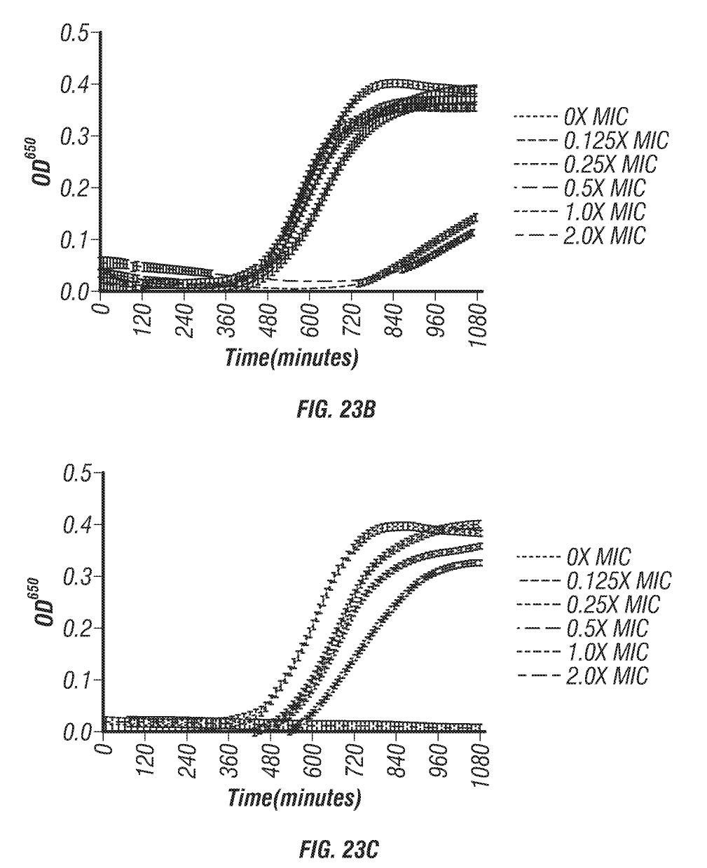

The rate of bacterial growth in presence of antimicrobial candidates, C58, C59, C59Na, or 4-76, was performed using stationary phase MRSA, USA 300 and SA LL06, or S. epidermidis, M0881, and compared with SoC antimicrobial, vancomycin. In addition, these growth rates were compared with the kill kinetics described earlier (FIGS. 4 & 5). Bacteria grown to stationary phase was then incubated with 0.125×, 0.25×, 0.5×, 1×, and 2×MIC concentrations of antimicrobial agents and absorbance measured at 650 nm (OD₆₅₀) at 10-min intervals for 18-h. Growth curves with MRSA strain USA 300 (FIGS. 21A-21D) demonstrate inhibition of bacteria at 1×MIC, and a dose response is observed with C59 and C59Na, but not with 4-76 and vancomycin. A reduction in bacterial growth rate, represented by a lower absorbance at OD₆₅₀, can be observed with C59 and C59Na at concentrations as low as 0.125×MIC compared with non-treated control. A growth curve with MRSA SA LL06 demonstrates similar results (FIGS. 22A-22C); C58 and C59 demonstrate a dose response with a reduction in bacterial growth rate at concentrations as low as 0.125×MIC. These results are similar to the kill kinetics results, in that C59 demonstrates a dose response, but vancomycin does not. However, in the kill kinetics experiments, vancomycin fails to eradicate stationary phase MRSA at concentrations up to 2×MIC. This anomaly can be explained by the different experimental conditions between the kill kinetics and growth curve experiments; bacterial samples are grown with constant shaking on an orbital shaker for a kill kinetics experiment but not for a growth curve experiment. Growth curve performed with S. epidermidis strain M0881 using log-phase mode bacteria demonstrates similar results to MRSA with C59 and vancomycin (FIGS. 23A-23C). At 1×MIC, the bacterial growth is inhibited, however, 4-76 show signs of growth (OD₆₅₀-0.1) between 10 and 18-h. These results differ from the MIC determination performed using the standard CLSI broth microdilution method and can be attributed to the higher sensitivity of optical density measurement using a plate reader for growth curve experiments, in contrast to the visual determination for growth for MIC determination.

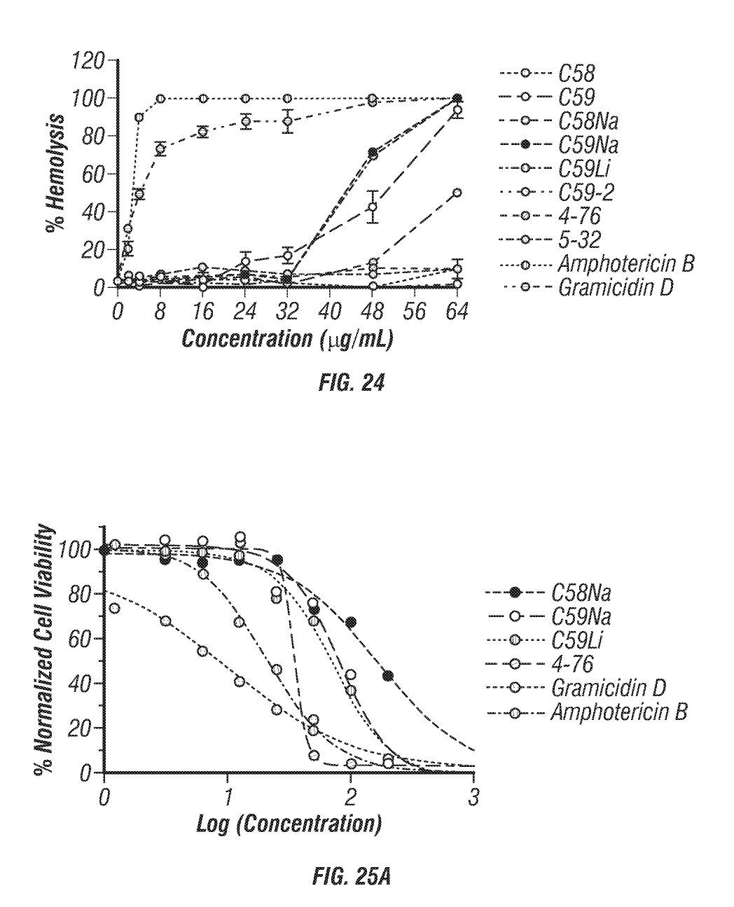

Toxicity of lead antimicrobial candidates was tested against erythrocytes, as well as eukaryotic epithelial and dermal cell lines in vitro. Hemolytic activity of lead candidates, C58, C58Na, C59, C59Na, C59Li, 4-76, and 5-32, as well as FDA approved membrane active agents, Gramicidin D and Amphotericin B was determined by incubating these compounds for 1-h with red blood cells (erythrocytes from sheep blood). Previously, hemolytic activity had been reported at 18-h, however, reviewing the literature revealed that most reported assays are performed with 1-h and 2-h time points. Hence, these experiments were repeated and determined hemolytic activity at a 1-h time point. At 1-h, C58, C59-2, 4-76, and 5-32 are found to be non-hemolytic and do not result in hemolysis at concentrations up to 64 μg/mL (FIG. 24). Additionally, C58Na, C59, C59Na, and C59Li are non-hemolytic at therapeutic concentrations with the onset of hemolysis occurring between 32 and 48 μg/mL for each of these compounds. In contrast, FDA-approved membrane active agents, Amphotericin B and Gramicidin D, demonstrate an onset of hemolysis at 2 μg/mL and result in 100% hemolysis at 8 and 48 μg/mL, respectively.

Next, the toxicity of lead candidates was tested against human lung airway epithelial cells (16HBE) and human dermal fibroblasts (HDF) using an alamarBlue® Cell Viability Assay. Cell viability was determined for C58Na, C59Na, C59Li, 4-76, Gramicidin D, and Amphotericin B relative to untreated controls (FIGS. 25A & 25B) and cell viability curves were analyzed using non-linear curve fit to yield the lethal dose at median cell viability (LD₅₀) for all cell lines (Table 8). These data demonstrate a lack of toxicity towards eukaryotic cells at concentrations that exert antimicrobial activity. The MIC₉₀ for these compounds ranges between 2 and 4 μg/mL and is at least three-fold lower than the LD₅₀ values. Thus, these compounds provide a large therapeutic window for treatment.

TABLE 8
Lethal dose at median cell viability (LD₅₀) after an 24-h
incubation with lead antimicrobials and FDA-
approved membrane active agents determined using non-linear
curve fit from the cell viability curves shown in FIGS. 25A & 25B.

| | 16HBE LD₅₀ (μg/mL) | Fibroblasts LD₅₀ (μg/mL) |
|---|---|---|
| C58Na | 92.3 | In progress |
| C59Na | 30 | Previously reported |
| C59Li | 24.4 | Previously reported |
| 4-76 | 18.1 | 22.2 |
| Gramicidin D | 20.5 | 3.9 |
| Amphotericin | 12.6 | 2.2 |

The potential of spontaneous resistance acquisition in MRSA strain, USA 300 with C59 and vancomycin, has been analyzed using a plate-based assay. An aliquot of 100 μL of bacterial suspension grown to stationary phase and adjusted to a density of 10¹¹ CFU/mL was spread onto agar plates supplemented with C59 or vancomycin at 1, 2, 4, and 8×MIC concentrations. The frequency of selection of resistant strains after a single-step exposure to antimicrobials is listed in Table 9. Preliminary results show bacterial colonies and hence, the risk of spontaneous resistance development, when stationary phase USA 300 is incubated with 2×, 4×, and 8×MIC of vancomycin for 72-h. In contrast, incubation with C59 does not result in colony formation at concentrations greater than 1×MIC.

TABLE 9
Frequency of resistance development with MRSA USA
300 after incubation with vancomycin or C59.

| Frequency of | | |
|---|---|---|
| Resistance Development | Vancomycin | C59 |
| 2× MIC | 1.83 × 10⁻⁸ ± 7.10 × 10⁻⁹ | No colonies |
| 4× MIC | 1.56 × 10⁻⁹ ± 7.12 × 10⁻¹⁰ | No colonies |
| 8× MIC | 5.77 × 10⁻¹⁰ ± 2.77 × 10⁻¹⁰ | No colonies |

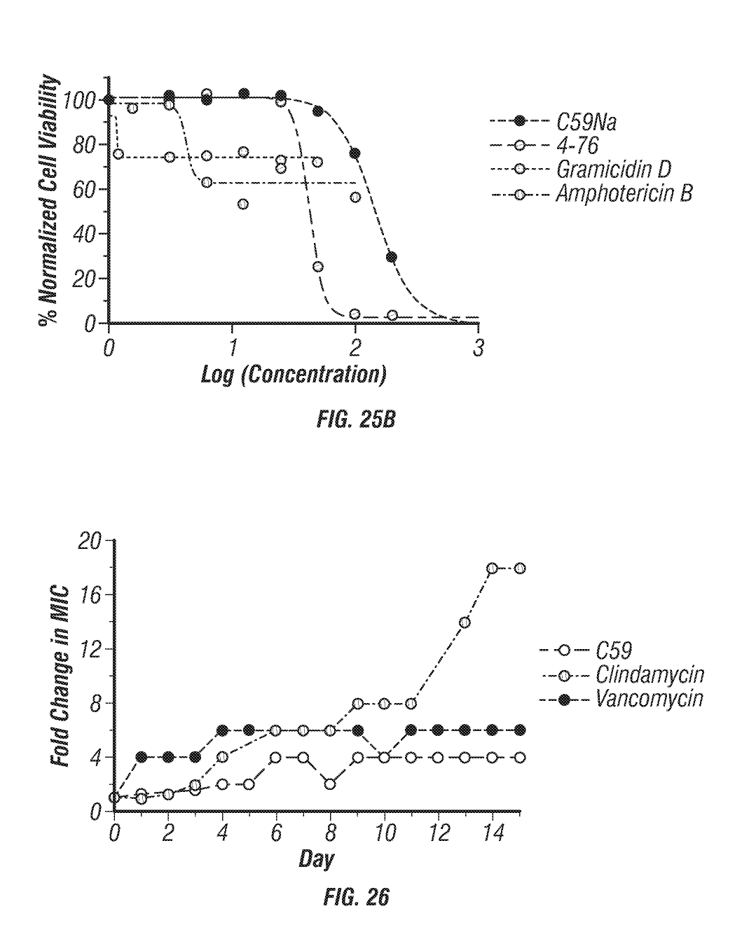

As a follow-up, the potential of MRSA to develop phenotypic resistance to C59 and control antimicrobials, vancomycin and clindamycin, was determined through sequential drug exposure studies. MRSA strain, USA 300, was grown to stationary phase and adjusted to a density of 10″ CFU/mL. A 100 μL aliquot of bacterial suspension was used to inoculate 10 mL antibiotic-free or antibiotic-supplemented Mueller-Hinton broth contained in glass vials. The antibiotic concentrations ranged from 4 doubling dilutions above to 3 below the MIC of each drug. Passages were performed every 24-h by transferring 1:1000 dilution from the second tube nearest the MIC for 15 days. Incubation with clindamycin results in an 18-fold increase in MIC after 15 days, while vancomycin and C59 incubation results in 6- and 4-fold increase in MIC, respectively (FIG. 26). These results further indicate a lower propensity of resistance acquisition for C59 over SoC antimicrobials, clindamycin and vancomycin.

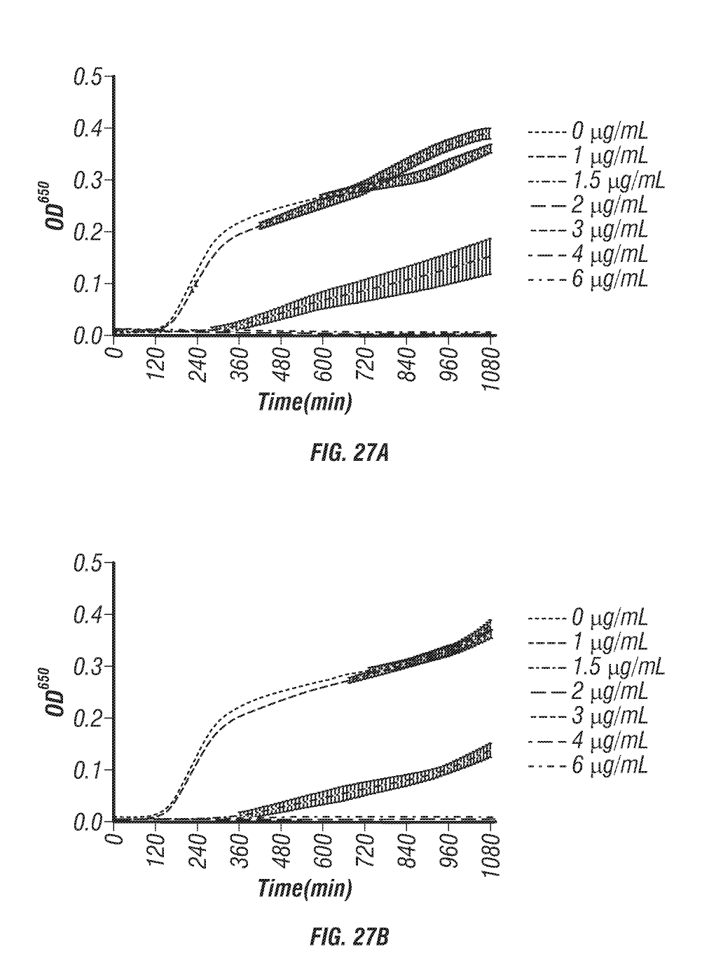

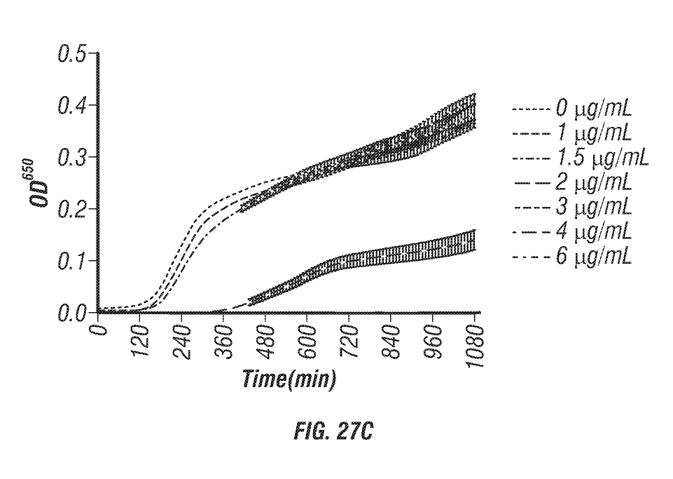

Finally, the antimicrobial efficacy of enantiomers of compound 4-76 were evaluated against log-phase MRSA strain USA 300, as described earlier. Results demonstrate that the enantiomer PK-1 has similar activity to 4-76, the parent compound, and is marginally superior to the enantiomer PK-2 (FIGS. 27A-27C). Both, PK-1 and 4-76 at a concentration of 1.5 μg/mL demonstrate lower bacterial growth rate represented by a lower absorbance at OD₆₅₀. Enantiomer PK-2 demonstrates similar activity at a concentration of 2 μg/mL.

### Example 4—Chemotherapeutic Activity

In addition to antimicrobial activity, preliminary screening of biaryl compounds with lung cancer cell lines demonstrates potential anticancer activity. The IC₅₀ values generated using a non-linear curve fit with lung cancer cell lines, A549, H460, H2122, and H2073 are listed in Table 10. Experiments were performed using C58, C58-2, C59, C59-2, C119, and acetylated C119 (C119-2). Biaryl compounds, specifically C119 demonstrates potential as an anticancer agent against both non-small cell lung cancer and large cell lung cancer cell lines. These results also favor acetylated versions over phenolic compounds. Additionally, these concentrations are clinically achievable for treatment of lung cancer and a detailed structure activity relationship experiments needs to be performed to identify promising anticancer candidates.

TABLE 10
Inhibitory concentration at median cell survival (LD₅₀) after a 72
hour incubation with biaryl compounds determined using non-linear
curve fit for lung cancer cell lines A549, H460, H2122, and H2073.

| | A549 | H460 | H2122 | H2073 |
|---|---|---|---|---|
| | LD₅₀ (μM) | LD₅₀ (μM) | LD₅₀ (μM) | LD₅₀ (μM) |
| C58 | 120 | 42 | 98 | 32 |
| C58-2 | 74 | 51 | 87 | 36 |
| C59 | 123 | 37 | 91 | 43 |
| C59-2 | 74 | 73 | 99 | 45 |
| C119 | 52 | 37 | 58 | 33 |
| C119-2 | TBD | TBD | TBD | 33 |

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference:
Anderson, N. G., Practical Process Research & Development—A Guide For Organic Chemists, 2^{nd} ed., Academic Press, New York, 2012.
Antibiotic Resistance Threats in the United States, 2013,
Antibiotic Resistance Threats in the United States. 2014 ed. www.cdc.gov/drugresistance/threat-report-2013/: Centers for Disease Control and Prevention; p. 114, 2013.
Austin-Ward and Villaseca, Rev. Med. Chil., 126(7):838-45, 1998.
Barber, J. Clin. Pathol., 14:385-393, 1961.
Bukowski et al., Clin. Cancer Res., 4(10):2337-47, 1998.
Christodoulides et al., Microbiology, 144(Pt 11):3027-37, 1998.
Davidson et al., J. Immunother., 21(5):389-98, 1998.
Dohi et al., Angew. Chem. Int. Ed. 50, 6142-6146, 2011.
Frisch, et al. Gaussian 09, revision B.01; Gaussian, Inc.: Wallingford, Conn., 2009.
Greene's Protective Groups in Organic Chemistry, Wuts and Greene, Ed., 1973
Hanibuchi et al., Int. J. Cancer, 78(4):480-485, 1998.
Hellstrand et al., Acta Oncol., 37(4):347-353, 1998.
Hui and Hashimoto, Infect. Immun., 66(11):5329-36, 1998.
Isnansetyo and Kamei, Antimicrobial Agents and Chemotherapy, 47:480-8, 2003.
Isnansetyo and Kamei, Antimicrobial Agents and Chemotherapy, 47:480-8, 2003.
Isnansetyo and Kamei, International Journal of Antimicrobial Agents, 34:131-5, 2009.
Isnansetyo and Kamei, International Journal of Antimicrobial Agents, 34:131-5, 2009.
Ito et al., J. Am. Chem. Soc. 2013, 135, 14078-14081.
Ito et al., J. Am. Chem. Soc., 135:14078-14081, 2013.
Jevons, British Med. J., 1:124-125, 1961.
Ju et al., Gene Ther., 7(19):1672-1679, 2000.
Kamei and Isnansetyo, International Journal of Antimicrobial Agents, 21:71-4, 2003.
Lambert and Danishefsky, J. Am. Chem. Soc., 128:426-427, 2006.
Leid et al., The Journal of Antimicrobial Chemotherapy, 67:138-48, 2012.
Livermore, Int. J. Antimicrob. Agents, 16(1:)S3-10, 2000.
March's Advanced Organic Chemistry: Reactions, Mechanisms, and Structure, 2007.
Marenich et al., J. Phys. Chem. B 2009, 113, 6378-6396.
Mitchell et al., Ann. NY Acad. Sci., 690:153-166, 1993.
Mitchell et al., J. Clin. Oncol., 8(5):856-869, 1990.
Morton et al., Arch. Surg., 127:392-399, 1992.
Nathan, Nature, 431:899-902, 2004.
Nicolaou et al., Angew. Chem. Int. Ed., 54:9203-9208, 2015.
Okuma et al., Chem. Commun., 48:11145-11147, 2012.
Olson et al., Can J Vet Res., 66:86-92, 2002.
Pangborn et al., Organometallics, 15:1518-1520, 1996.
Pietras et al., Oncogene, 17(17):2235-49, 1998.
Qin et al., Proc. Natl. Acad. Sci. USA, 95(24):14411-14416, 1998.
Raad et al., Antimicrobial Agents and Chemotherapy, 51:1656-60, 2007.
Radovic-Moreno et al., ACS Nano, 6:4279-87, 2012.
Ravindranath and Morton, Intern. Rev. Immunol., 7: 303-329, 1991.
Remington's Pharmaceutical Sciences, 15^{th} Ed., 1035-1038 and 1570-1580, 1990.
Remington's Pharmaceutical Sciences, 15^{th} Ed., 3:624-652, 1990.
Rose and Poppens, The Journal of Antimicrobial Chemotherapy, 63:485-8, 2009.
Rosenberg et al., Ann. Surg. 210(4):474-548, 1989.
Rosenberg et al., N. Engl. J. Med., 319:1676, 1988.
Still et al., J. Org. Chem., 43:2923-2925, 1978.
U.S. Pat. No. 5,739,169
U.S. Pat. No. 5,801,005
U.S. Pat. No. 5,824,311
U.S. Pat. No. 5,830,880
U.S. Pat. No. 5,846,945
US Department of Health and Human Services, Center of Disease Control and Prevention, WHO. Antimicrobial resistance: global report on surveillance. In: Resistance D, editor. www.who.int/drugresistance/documents/surveillancereport/en/: World Health Organization; p. 257, 2014.
Wright et al., Dalton Transactions, 41:6500-6, 2012.
www.cdc.gov/drugresistance/threat report-2013/pdf/ar-threats-2013-508.pdf.
Zhang et al., Angew. Chem. Int. Ed, 52, 1753-1757, 2013.
Zhao and Truhlar, Acc. Chem. Res. 2008b, 41, 157-167.
Zhao and Truhlar, Theor. Chem. Acc. 2008a, 120, 215-241.

1. A compound of the formula: 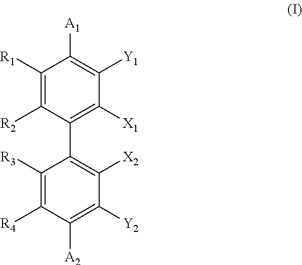
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
Y₂ is hydrogen or halo;
A₁ and A₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₁ and R₂ are taken together and are alkenediyl_{(C≤8)} or substituted alkenediyl_{(C≤8)}; and
R₃ and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₃ and R₄ are taken together and are alkenediyl_{(C≤8)} or substituted alkenediyl_{(C≤8)}; or
a compound of the formula: 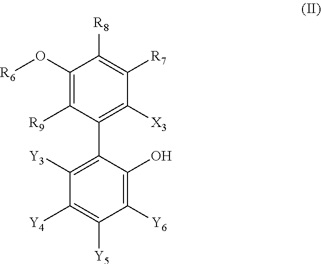
wherein:
X₃ is —OR_{d} or —NRₑR_{f}, wherein:
R_{d} is hydrogen, alkyl_{(C≤8)}, cycloalkyl_{(C≤8)}, acyl_{(C≤8)}, or a substituted version of any of these groups; or a hydroxy protecting group;
Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
or a pharmaceutically acceptable salt thereof.

2. The compound of claim 1, wherein the formula is further defined as: 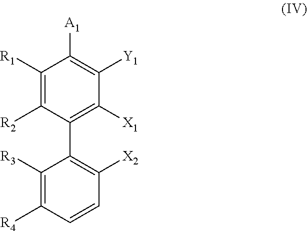
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₁ and R₂ are taken together and are alkenediyl_{(C≤8)} or substituted alkenediyl_{(C≤8)}; and
R₃ and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; or R₃ and R₄ are taken together and are alkenediyl_{(C≤8)} or substituted alkenediyl_{(C≤8)};
or a pharmaceutically acceptable salt thereof.

3. The compound of claim 1, wherein the formula is further defined as: 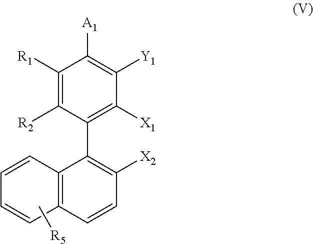
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; or R₁ and R₂ are taken together and are alkenediyl_{(C≤8)} or substituted alkenediyl_{(C≤8)}; and
R₅ is hydrogen, halo, methoxy, ethoxy, methyl, or ethyl;
or a pharmaceutically acceptable salt thereof.

4. The compound of claim 1, wherein the formula is further defined as: 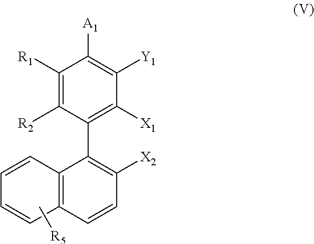
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, or substituted cycloalkyl_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; and
R₅ is hydrogen, halo, methoxy, ethoxy, methyl, or ethyl;
or a pharmaceutically acceptable salt thereof.

5. The compound of claim 1, wherein the formula is further defined as: 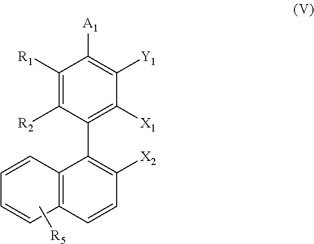
wherein:
X₁ is amino or alkylamino_{(C≤8)}, substituted alkylamino_{(C≤8)}, dialkylamino_{(C≤8)}, or substituted dialkylamino_{(C≤8)};
X₂ is hydroxy;
Y₁ is halo;
A₁ is hydrogen, halo, alkyl_{(C≤8)}, substituted alkyl_{(C≤8)}, cycloalkyl_{(C≤8)}, or substituted cycloalkyl_{(C≤8)};
R₁ and R₂ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, substituted alkoxy_{(C≤8)}; and
R₅ is hydrogen, halo, methoxy, ethoxy, methyl, or ethyl;
or a pharmaceutically acceptable salt thereof.

6. The compound of claim 1, wherein the formula is further defined as: 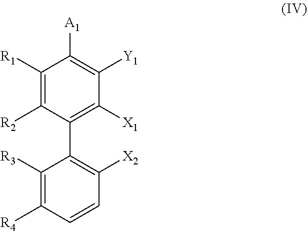
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, R₃, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
or a pharmaceutically acceptable salt thereof.

7. The compound of claim 1, wherein the formula is further defined as: 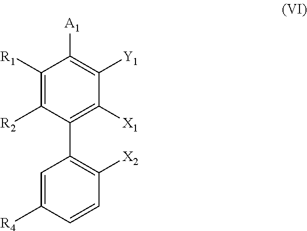
wherein:
X₁ is amino or alkylamino_{(C≤8)}, dialkylamino_{(C≤8)}, amido_{(C≤8)}, or a substituted version of any of these groups; or —N(Rₐ)R_{b}, wherein:
Rₐ is a monovalent amino protecting group or is taken together with R_{b} and form a divalent amino protecting group; and
R_{b} is hydrogen, a monovalent amino protecting group; or is taken together with Rₐ and form a divalent amino protecting group;
X₂ is hydroxy or alkoxy_{(C≤8)}, acyloxy_{(C≤8)}, or a substituted version of any of these groups; or —OR_{c} wherein:
R_{c} is a hydroxy protecting group;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
or a pharmaceutically acceptable salt thereof.

8. The compound of claim 1, wherein the formula is further defined as: 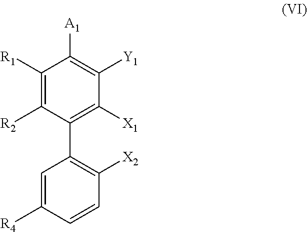
wherein:
X₁ is amino or alkylamino_{(C≤8)}, substituted alkylamino_{(C≤8)}, dialkylamino_{(C≤8)}, or substituted dialkylamino_{(C≤8)};
X₂ is hydroxy;
Y₁ is halo;
A₁ is hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)}; and
R₁, R₂, and R₄ are each independently hydrogen, halo, alkyl_{(C≤8)}, substituted alkyl_{(C≤8)}, cycloalkyl_{(C≤8)}, substituted cycloalkyl_{(C≤8)}, alkoxy_{(C≤8)}, or substituted alkoxy_{(C≤8)};
or a pharmaceutically acceptable salt thereof.

9. The compound of claim 1, wherein the formula is further defined as: 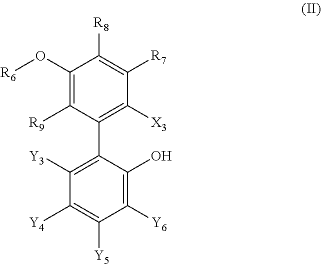
wherein:
X₃ is —OR_{d} or —NRₑR_{f}, wherein:
R_{d} is hydrogen, alkyl_{(C≤8)}, cycloalkyl_{(C≤8)}, acyl_{(C≤8)}, or a substituted version of any of these groups; or a hydroxy protecting group;
Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
or a pharmaceutically acceptable salt thereof.

10. The compound of claim 1, wherein the formula is further defined as: 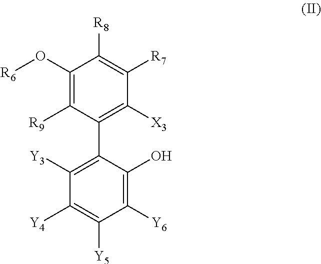
wherein:
X₃ is —OR_{d} or —NRₑR_{f}, wherein:
R_{d} is hydrogen, alkyl_{(C≤8)}, cycloalkyl_{(C≤8)}, acyl_{(C≤8)}, or a substituted version of any of these groups; or a hydroxy protecting group;
Rₑ and R_{f} are each independently hydrogen, alkyl_{(C≤8)}, cycloalkyl_{(C≤8)}, acyl_{(C≤8)}, or a substituted version of any of these groups; or a monovalent amino protecting group, or Rₑ and R_{f} are taken together and are a divalent amino protecting group;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; or R₇ and R₈ are taken together and are alkenediyl_{(C≤8)} or substituted alkenediyl_{(C≤8)}; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)}, or Y₄ and Y₅ are taken together and are alkenediyl_{(C≤8)} or substituted alkenediyl_{(C≤8)}, or Y₅ and Y₆ are taken together and are alkenediyl_{(C≤8)} or substituted alkenediyl_{(C≤8)};
or a pharmaceutically acceptable salt thereof.

11. The compound according to claim 1, wherein the formula is further defined as: 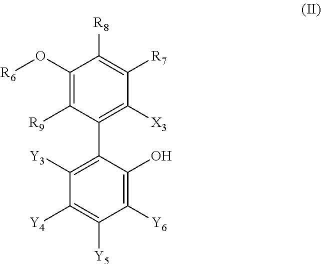
wherein:
X₃ is —OR_{d} or —NRₑR_{f}, wherein:
R_{d} is hydrogen, alkyl_{(C≤8)}, cycloalkyl_{(C≤8)}, acyl_{(C≤8)}, or a substituted version of any of these groups;
Rₑ and R_{f} are each independently hydrogen, alkyl_{(C≤8)}, cycloalkyl_{(C≤8)}, acyl_{(C≤8)}, or a substituted version of any of these groups;
R₆ is hydrogen or alkyl_{(C≤8)}, cycloalkyl_{(C≤8)}, or a substituted version of either of these groups;
R₇, R₈, and R₉ are each independently hydrogen, halo, or alkyl_{(C≤8)}, cycloalkyl_{(C≤8)}, alkoxy_{(C≤8)}, or a substituted version of any of these groups; and
Y₃, Y₄, Y₅, and Y₆ are each independently hydrogen, halo, hydroxy, or alkyl_{(C≤8)}, cycloalkyl_{(C≤8)}, alkoxy_{(C≤8)}, or a substituted version of any of these groups; or Y₃ and Y₄ are taken together and are alkenediyl_{(C≤8)} or substituted alkenediyl_{(C≤8)};
or a pharmaceutically acceptable salt thereof.

12-125. (canceled)

126. The compound according to claim 1, wherein the compound is further defined as: 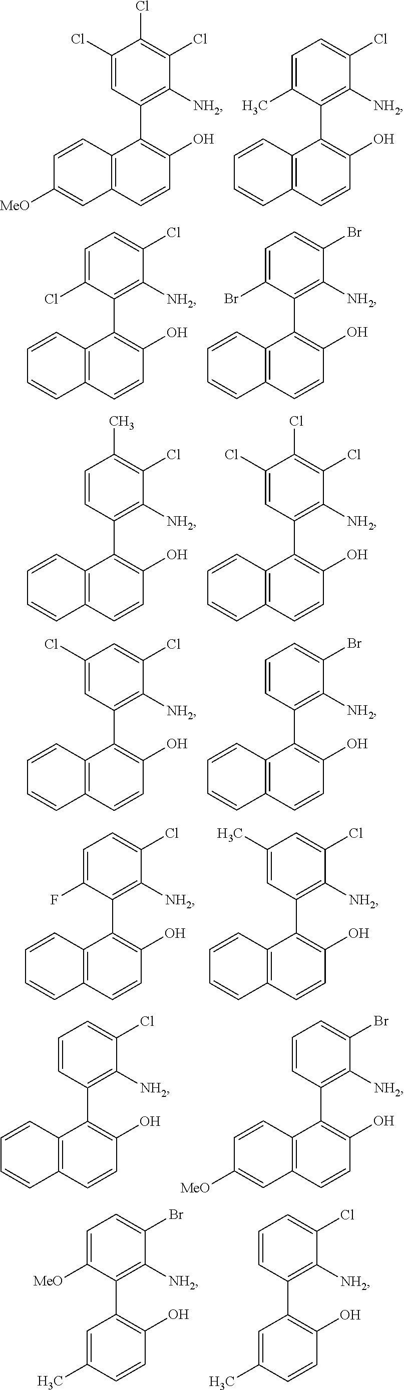 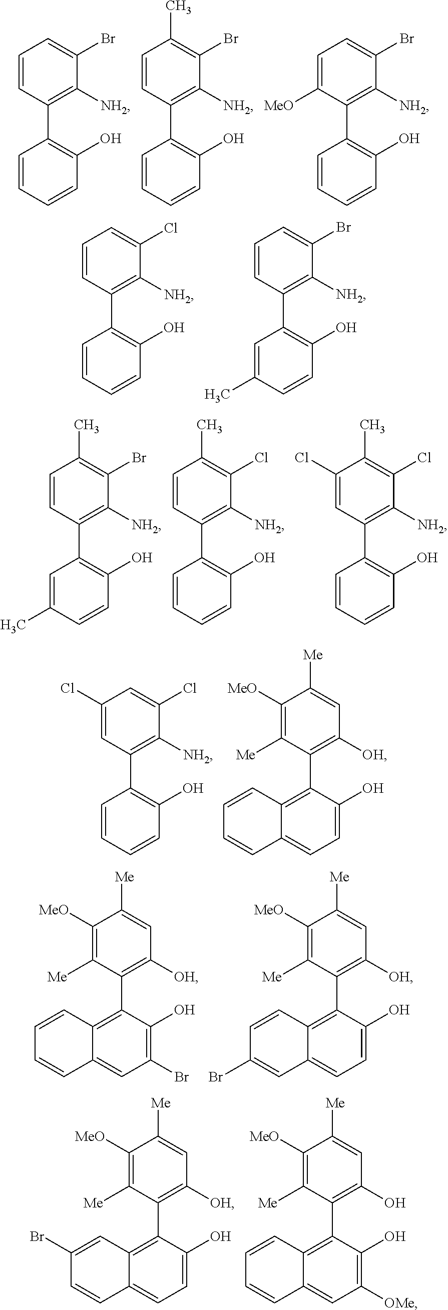 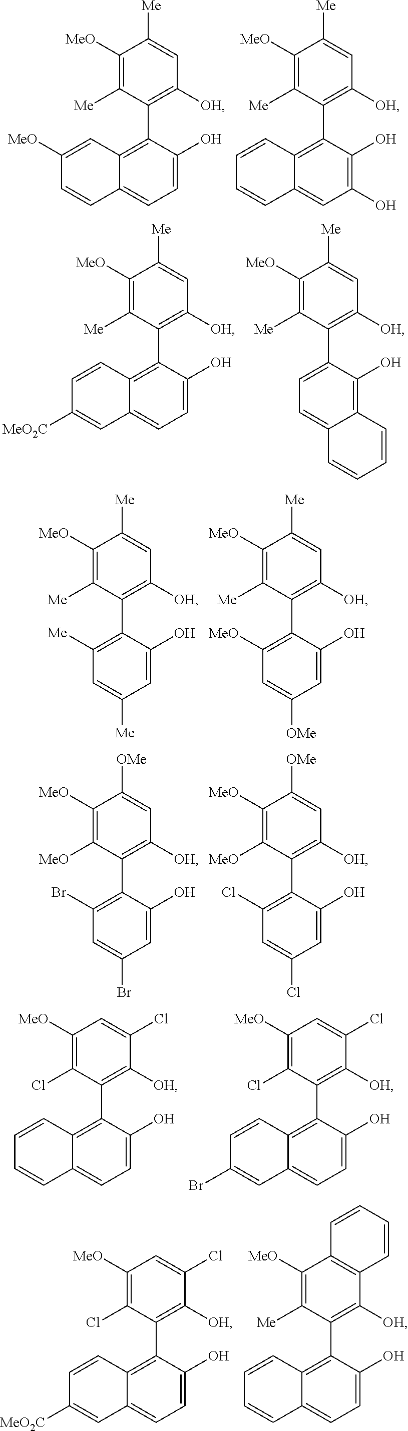 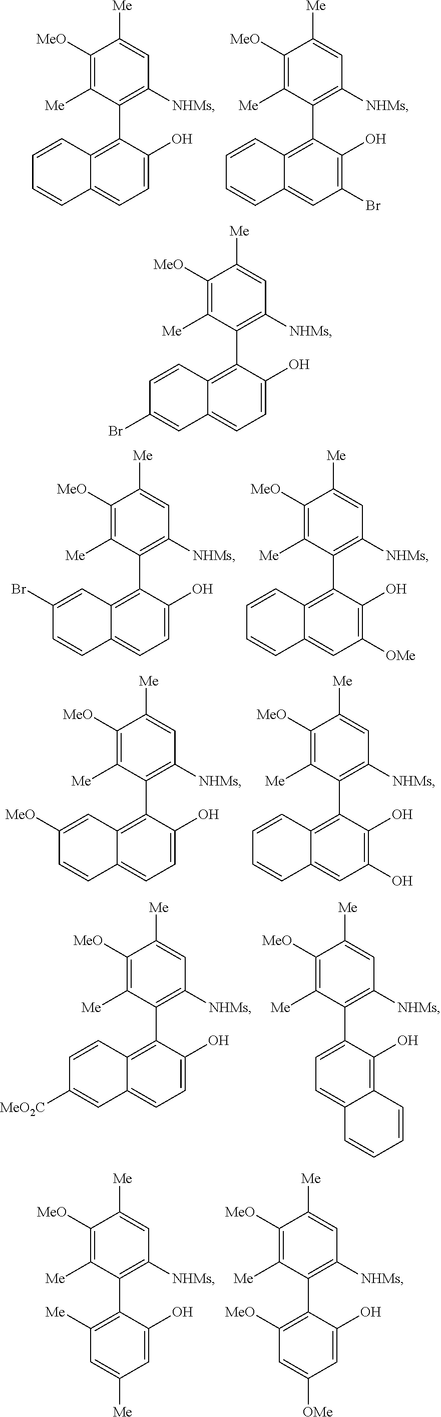 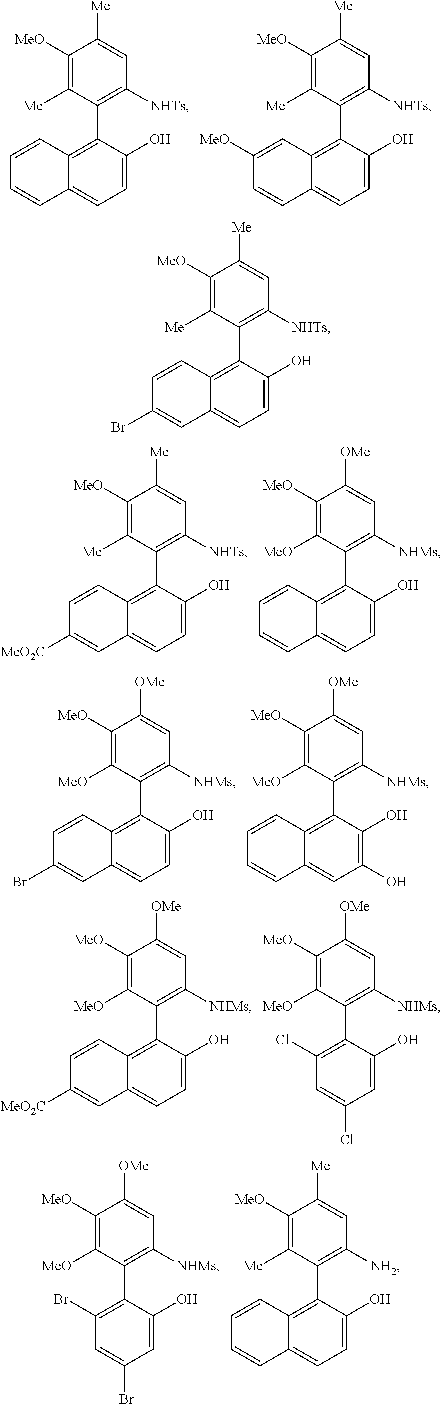 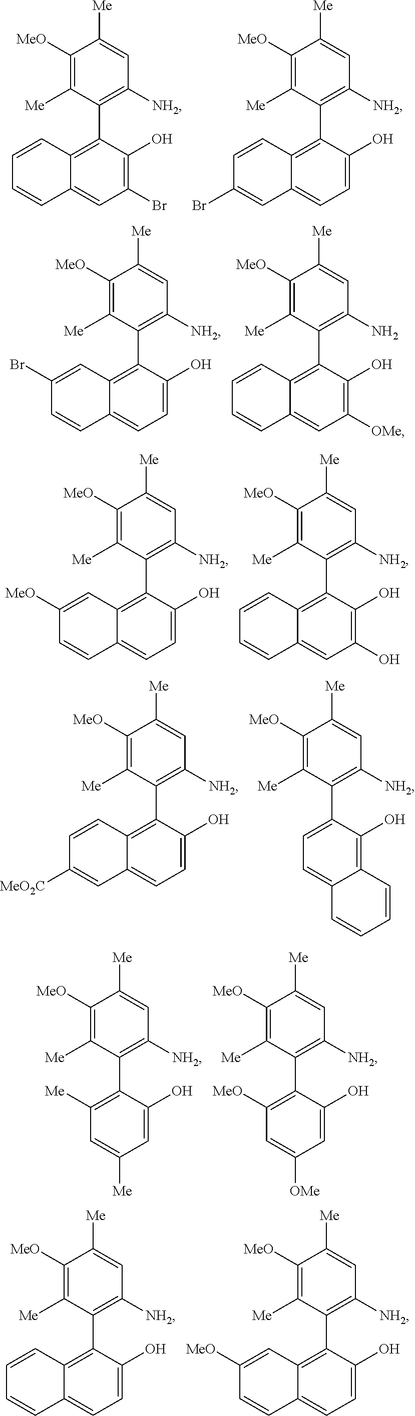 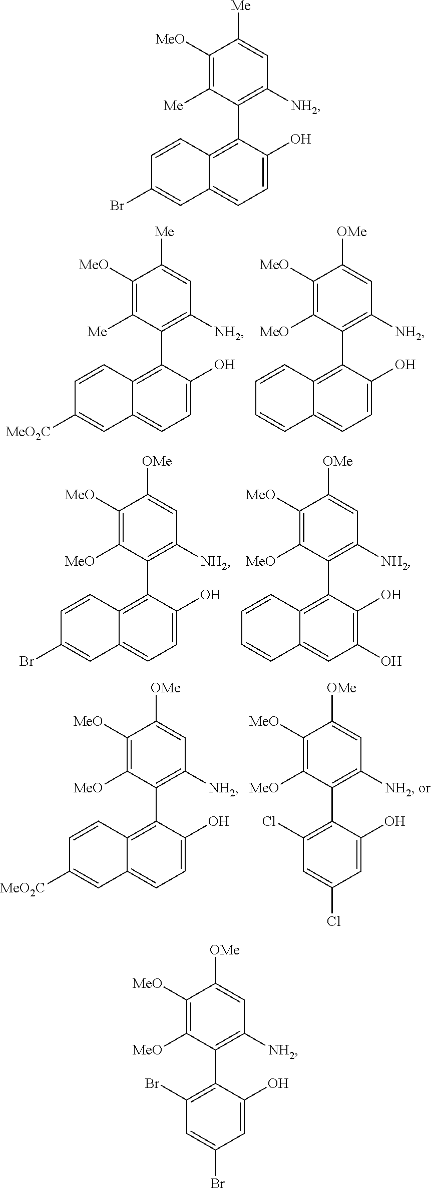
or a pharmaceutically acceptable salt thereof.

127. A pharmaceutical composition comprising:
(a) a compound of claim 1; and
(b) a pharmaceutically acceptable carrier.

128-133. (canceled)

134. A pharmaceutical composition comprising:
(a) a compound of the formula: 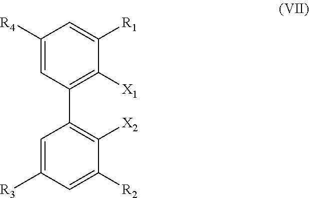
wherein:
R₁, R₂, R₃, and R₄ are each halo;
X₁ and X₂ are OAc or O⁻M⁺; wherein:
M⁺ is a monovalent cations or the M⁺ associated with X₁ and X₂ are taken together and are a divalent cation; and
(b) a pharmaceutically acceptable carrier.

135. The pharmaceutical composition of claim 134, wherein M⁺ is a monovalent cation.

136. (canceled)

137. (canceled)

138. The pharmaceutical composition of claim 134, wherein R₁, R₂, R₃, and R₄ are all chloro.

139. The pharmaceutical composition of claim 134, wherein R₁, R₂, R₃, and R₄ are all bromo.

140. The pharmaceutical composition of claim 134, wherein the compound is further defined as:

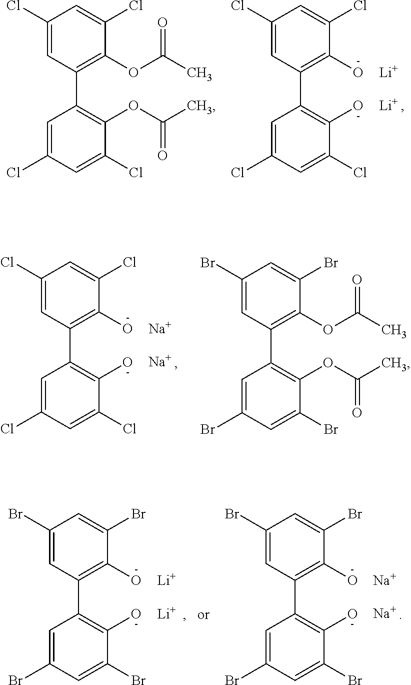

141-146. (canceled)

147. A method of treating a disease or disorder in a patient comprising administering to the patient in need thereof a therapeutically effective amount of a compound or composition of claim 1.

148-180. (canceled)
